(19) United States
(12) Patent Application Publication
(10) Pub. No.: US 2024/0318169 A1
(43) Pub. Date: Sep. 26, 2024

(54) PHOTOLABILE SPATIAL LABEL GENERATION

(71) Applicant: Ultima Genomics, Inc., Fremont, CA (US)

(72) Inventors: Ron Saar Dover, Palo Alto, CA (US); Zohar Shipony, Rehovot (IL)

(73) Assignee: Ultima Genomics, Inc., Fremont, CA (US)

(21) Appl. No.: 18/737,499

(22) Filed: Jun. 7, 2024

Related U.S. Application Data
(63) Continuation of application No. PCT/US2022/081962, filed on Dec. 19, 2022.
(60) Provisional application No. 63/291,608, filed on Dec. 20, 2021.

(51) Int. Cl. C12N 15/10

(52) U.S. Cl. CPC C12N 15/1065; C12N 15/1068

(57) ABSTRACT

Disclosed herein are method for labeling analytes using spatial barcodes, illumination, and/or photolabile cross-linkers. The methods may generate a set of unique spatial barcodes using limited sets differential nucleotide sequences. The labeling may identify a spatial location of an analyte. Also provided herein are compositions, kits, or system for carrying out the methods.

### CROSS-REFERENCE

This application claims the benefit of U.S. Provisional Pat. App. No. 63/291,608, filed Dec. 20, 2021, which is entirely incorporated by reference herein.

### BACKGROUND

Biological sample processing has various applications in the fields of molecular biology and medicine (e.g., diagnosis). For example, biological sample processing may be used to label analytes in a biological sample for diagnosis of a certain condition in a subject and in some cases formation of a treatment plan. Analyte labeling is widely used for molecular biology applications, including enrichment, depletion, identification, or visualization of various analytes in a biological sample. Biological sample processing may involve a fluidics system and/or a detection system.

Despite the advance of biological sample processing technology, labeling analytes with sufficient diversity to be useful for subsequent identification still requires laborious efforts.

### SUMMARY

In some cases, a label for an analyte may comprise a nucleic acid sequence that can be read in a downstream sequencing operation. A label may encode and/or be unique to a certain characteristic or property (e.g., sample origin, cell origin, nucleus origin, spatial location, reaction condition, etc.) which can later be attributed to an analyte tagged by the label. In application, two analytes tagged by different labels may be distinguished based on the label (e.g., identified to have different sample origins, identified to have different cell or nucleus origins, identified to have come from different spatial locations, identified to be treated with different reaction conditions, etc.), and similarly two analytes tagged by the same label may be associated together (e.g., identified to have a same sample origin, identified to have a same cell or nucleus origin, identified to have come from a same spatial location, identified to be treated with a same reaction condition, etc.). In order to properly encode many characteristics or properties, and/or ensure uniqueness of a label to a certain characteristic or property, a set of labels needs to have sufficient diversity.

To generate a diverse set of nucleic acid barcodes, a method of combinatorial chemistry and split-pooling can be used, in which, generally, (i) at a first round, i.e., n=1, 1^{st} barcode segments are distributed to each of a set of x containers, such that each container receives a 1^{st} set of barcode segments unique to the container amongst the set of x containers, where optionally the 1^{st} set of barcode segments are attached to a substrate that is to be barcoded (e.g., an analyte, a barcode-carrying vehicle, such as a bead, etc.), (ii) the contents of all of the containers are pooled and randomly split into the set of x containers, (iii) (n+1)^{th} barcode segments are distributed to each of the set of x containers, such that each container receives a (n+1)^{th} set of barcode segments unique to the container amongst the set of x containers, (iv) within each container, combining (e.g., ligating) the different barcode segments to generate combined barcode segments each comprising 1^{st} through n^{th} barcode segments and the newest added (n+1)^{th} barcode segment (e.g., when n=4, the combined barcode segments each comprises the 1^{st}, 2^{nd}, 3^{rd}, 4^{th}, and 5^{th} barcode segments), and (v) repeating (ii)-(iv) where n=n+1 at the start of each repeat, for n iterations to generate a final set of barcodes which has an estimated diversity of xⁿ⁺¹ different barcodes. In an example, performing the above method using a 96 well plate as the set of containers and performing only 3 rounds of split-pooling would generate an estimated diversity of 96⁴=84,934,656 different barcodes. The generated set of barcodes may be used to tag analytes. However, the repeated operations of splitting and pooling barcode components may introduce many errors and involve loss of valuable resources. Further, although the final products have diversity, their respective identities may be unknown (which barcodes are in which container), limiting their use in downstream operations.

Recognized herein is a need for efficient labeling of analytes. Recognized herein is a need for spatial barcodes for labeling analytes, in which the spatial barcodes encode and/or are unique to distinct spatial locations. Recognized herein is a need for labelling pre-determined or individual spatial locations of a substrate, or analytes associated thereto. Recognized herein is a need for generating a diverse set of barcodes without requiring physical splitting and pooling steps, such as by using light. Recognized herein is a need for generating a diverse set of barcodes, where identities and locations of the individual barcodes are known. Provided herein are methods, systems, compositions, and kits that address at least the abovementioned needs.

The provided systems, methods, kits, and compositions allow for the generation of unique spatial barcodes at pre-determined spatial locations of a substrate, directed by iterative cycles of selective illumination on the substrate. In some cases, the spatial barcodes may be generated on a surface of the substrate at such spatial locations. Downstream, the surface may subsequently receive a sample comprising one or more analytes such that the spatial barcodes can contact and tag the analytes. In some cases, the spatial barcodes may be released from the surface subsequent to contacting and/or tagging the analytes. In other cases, the spatial barcodes may be released from the surface prior to contacting and/or tagging the analytes, such as to diffuse into a sample (e.g., tissue sample) to tag an analyte in the sample. Alternatively or in addition, the generated spatial barcodes may be released from the surface, collected, and contact and tag one or more analytes off the substrate, such as in another reaction environment. In other cases, the spatial barcodes may not be released from the surface subsequent to contacting and/or tagging the analytes, such that the tagged analytes are immobilized to the substrate. In other cases, the spatial locations on the substrate may have immobilized thereto one or more analytes, and the spatial barcodes may be generated directly on the one or more analytes at such spatial locations. In some cases, the identity and location of the spatial barcode on the substrate can be determined by tracing (i) the known identity of the sets of unique sequences added and (ii) the location of selective illumination, during each cycle of selective illumination. Beneficially, a diverse set of barcodes may be generated without the need to perform physical split and pool operations. Beneficially, the identities and locations of the final barcodes generated may be known. Beneficially, in cases where the final spatial barcodes are generated directly on the analytes, a separate tagging step may be unneeded. This is advantageous over other systems which assay analytes using an array of probes or other barcodes on a substrate, in which the system requires deliberate generation of such array with known locations or in which the system requires a pre-determination step to determine the identities and locations of the probes or other barcodes in the array.

In an aspect, provided is a method, comprising: (a) providing a substrate, wherein the substrate comprises a plurality of individually addressable locations comprising a plurality of first polynucleotides immobilized thereto; (b) applying a fluid layer to the substrate, wherein the fluid layer comprises (i) a plurality of second polynucleotides and (ii) a plurality of ligation templates, under conditions sufficient to hybridize the plurality of first polynucleotides to the plurality of ligation templates and to hybridize the plurality of second polynucleotides to the plurality of ligation templates, wherein the fluid layer has a thickness of at most 50 micrometers; and (c) subsequent to hybridization in (b), selectively illuminating a subset of the plurality of individually addressable locations, under conditions sufficient to: (i) link a subset of the plurality of first polynucleotides at the subset of the plurality of individually addressable locations with a subset of the plurality of ligation templates, and (ii) link a subset of the plurality of second polynucleotides with the subset of the plurality of ligation templates.

In some embodiments, the method further comprises: (d) subsequent to (c), removing a plurality of non-linked second polynucleotides or a plurality of non-linked ligation templates from the substrate. In some embodiments, the removing in (d) comprises providing to the substrate a solution comprising dimethyl sulfoxide (DMSO), sodium hydroxide (NaOH), or formamide. In some embodiments, the solution comprises the DMSO at least about 1% by volume in the solution. In some embodiments, the method further comprises, subsequent to (d), washing the substrate to remove the solution from the substrate.

In some embodiments, the subset of the plurality of individually addressable locations is selected based on a pre-determined spatial location of the substrate corresponding to the subset of the plurality of individually addressable locations. In some embodiments, (c) comprises using a Digital Micromirror Device (DMD) to address the pre-determined spatial location.

In some embodiments, the method further comprises, subsequent to linking in (c), (e) subjecting the substrate to conditions sufficient for the subset of the plurality of first polynucleotides and the subset of the plurality of second polynucleotides to form a bond. In some embodiments, a ligase catalyzes coupling of the subset of the plurality of first polynucleotides and the subset of the plurality of second polynucleotides. In some embodiments, subsequent to (e), phosphodiester bonds are formed between the subset of the plurality of first polynucleotides and the subset of the plurality of second polynucleotides.

In some embodiments, the method further comprises, subsequent to (e), performing an amplification reaction to generate a plurality of amplification products of the subset of the plurality of first polynucleotides coupled to the subset of the plurality of second polynucleotides. In some embodiments, the method further comprises sequencing the plurality of amplification products, or derivatives thereof.

In some embodiments, the selectively illuminating in (c) comprises providing ultraviolet (UV) light. In some embodiments, the UV light comprises a wavelength of about 365 nanometers (nm). In some embodiments, the selectively illuminating in (c) comprises providing UV light for at most about 1 minute.

In some embodiments, the method further comprises, subsequent to the selective illumination in (c), subjecting the plurality of first polynucleotides, the plurality of second polynucleotides, and the plurality of ligation templates to an additional illumination, under conditions sufficient to break a subset of a plurality of links generated in (c) between (i) the subset of the plurality of first polynucleotides and the subset of the plurality of ligation templates, or (ii) the subset of the plurality of second polynucleotides and the subset of the plurality of ligation templates.

In some embodiments, the additional illumination comprises UV light. In some embodiments, the UV light comprises a wavelength of about 312 nanometers (nm).

In some embodiments, a plurality of analytes are immobilized to the plurality of individually addressable locations, and wherein the plurality of first polynucleotides are coupled to the plurality of analytes. In some embodiments, the method further comprises, prior to (a), (i) providing the substrate, (ii) immobilizing the plurality of analytes at the plurality of individually addressable locations, and (iii) coupling the plurality of first polynucleotides to the plurality of analytes.

In some embodiments, the plurality of first polynucleotides is coupled to the plurality of analytes via a plurality of analyte-binding moieties of the plurality of first polynucleotides. In some embodiments, the plurality of analyte-binding moieties comprises comprise one or more members selected from the group consisting of: nucleic acids, proteins, lipids, saccharides, polysaccharides, and a combination thereof. In some embodiments, the proteins comprise antibodies or antigen binding fragments thereof.

In some embodiments, the plurality of analytes comprises one or more members selected from the group consisting of: nucleic acids, proteins, cells, lipids, saccharides, polysaccharides, and a combination thereof. In some embodiments, the proteins comprise one or more of extracellular proteins, cell surface proteins, cell membrane proteins, and intracellular proteins.

In some embodiments, a plurality of beads are immobilized to the plurality of individually addressable locations, and wherein the plurality of first polynucleotides are coupled to the plurality of beads.

In some embodiments, (c) comprises (i) cross-linking the subset of the plurality of first polynucleotides with the subset of the plurality of ligation templates, and (ii) cross-linking the subset of the plurality of second polynucleotides with the subset of the plurality of ligation templates.

In some embodiments, the plurality of first polynucleotides comprises a plurality of cross-linkers used in the cross-linking in (c)(i). In some embodiments, the plurality of cross-linkers comprises 3-Cyanovinylcarbazole nucleosides (CNVKs).

In some embodiments, the cross-linking in (c)(i) comprises forming a cross-link between a cross-linker of the plurality of cross-linkers and a nucleotide of the plurality of ligation templates. In some embodiments, the nucleotide of the plurality of ligation templates is a cytosine (C) or a thymine (T). In some embodiments, the nucleotide of the plurality of ligation templates is a C. In some embodiments, the nucleotide of the plurality of ligation templates is a T.

In some embodiments, (c) comprises (i) linking, via non-hydrogen bonds, the subset of the plurality of first polynucleotides with the subset of the plurality of ligation templates, and (ii) linking, via non-hydrogen bonds, the subset of the plurality of second polynucleotides with the subset of the plurality of ligation templates. In some embodiments, the non-hydrogen bonds comprise one or more of a covalent bond, cycloaddition bond, and photocycloaddition bond.

In some embodiments, a first polynucleotide of the plurality of first polynucleotides comprises a first barcode sequence, and wherein a second polynucleotide of the plurality of second polynucleotides comprises a second barcode sequence different from the first barcode sequence.

In some embodiments, a second polynucleotide of the plurality of second polynucleotides comprises a barcode sequence.

In some embodiments, the fluid layer has a thickness of at most about 15 micrometers.

In another aspect, provided is a method, comprising: (a) providing a substrate, wherein the substrate comprises a plurality of individually addressable locations comprising a plurality of first polynucleotides immobilized thereto; (b) applying a first fluid layer to the substrate, wherein the first fluid layer comprises (i) a plurality of second polynucleotides comprising a first barcode sequence, and (ii) a first plurality of ligation templates, wherein the first fluid layer has a thickness of at most 50 micrometers; (c) subjecting a first subset of the plurality of individually addressable locations to selective illumination, under conditions sufficient to: (i) link a subset of the plurality of first polynucleotides at the first subset of the plurality of individually addressable locations with a subset of the first plurality of ligation templates hybridized thereto, and (ii) link a subset of the plurality of second polynucleotides with the subset of the first plurality of ligation templates hybridized thereto; (d) applying a second fluid layer to the substrate, wherein the second fluid layer comprises (i) a plurality of third polynucleotides comprising a second barcode sequence, and (ii) a second plurality of ligation templates, wherein the second fluid layer has a thickness of at most 15 micrometers; and (c) subjecting a second subset of the plurality of individually addressable locations to selective illumination, under conditions sufficient to: (i) link a second subset of the plurality of second polynucleotides at the second subset of the plurality of individually addressable locations with a subset of the second plurality of ligation templates hybridized thereto, and (ii) link a subset of the plurality of third polynucleotides with the subset of the second plurality of ligation templates hybridized thereto.

In some embodiments, the first plurality of ligation templates and the second plurality of ligation templates have identical sequences. In some embodiments, first plurality of ligation templates and the second plurality of ligation templates comprise different sequences.

In some embodiments, the first barcode sequence and the second barcode sequence comprise sequence homology or identity. In some embodiments, the first barcode sequence and the second barcode sequence comprise different sequences.

In some embodiments, the first subset of the plurality of individually addressable locations and the second subset of the plurality of individually addressable locations are mutually exclusive locations. In some embodiments, the first subset of the plurality of individually addressable locations and the second subset of the plurality of individually addressable locations are a same set of individually addressable locations. In some embodiments, the first subset of the plurality of individually addressable locations and the second subset of the plurality of individually addressable locations comprises at least a common subset of individually addressable locations.

In some embodiments, the first subset of the plurality of individually addressable locations is selected based on a pre-determined spatial location of the substrate corresponding to the first subset of the plurality of individually addressable locations. In some embodiments, the second subset of the plurality of individually addressable locations is selected based on a second pre-determined spatial location of the substrate corresponding to the second subset of the plurality of individually addressable locations. In some embodiments, the pre-determined spatial location or the second pre-determined spatial location of the substrate is addressed by a Digital Micromirror Device (DMD).

In some embodiments, the method further comprises, subsequent to linking in (c), subjecting the substrate to second conditions sufficient for the subset of the plurality of first polynucleotides and the subset of the plurality of second polynucleotides to form a bond. In some embodiments, the method further comprises, subsequent to linking in (c), subjecting the substrate to second conditions sufficient for the second subset of the plurality of second polynucleotides and the subset of the plurality of third polynucleotides to form a bond. In some embodiments, formation of the bond is catalyzed by a ligase. In some embodiments, the bond is a phosphodiester bond.

In some embodiments, the method further comprises, subsequent to the formation of the bond, performing an amplification reaction to generate a plurality of amplification products. In some embodiments, the method further comprises sequencing the plurality of amplification products, or derivatives thereof.

In some embodiments, the selective illumination in (c) and (e) comprises providing ultraviolet (UV) light. In some embodiments, the UV light comprises a wavelength of about 365 nanometers (nm).

In some embodiments, the method further comprises, subsequent to (c), subjecting the plurality of first polynucleotides, the plurality of second polynucleotides, and the first plurality of ligation templates to a second illumination, under conditions sufficient to break a subset of a plurality of links generated in (c) between (i) the subset of the plurality of first polynucleotides and the subset of the first plurality of ligation templates, and (ii) the subset of the plurality of second polynucleotides and the subset of the second plurality of ligation templates.

In some embodiments, the method further comprises, subsequent to (e), subjecting the plurality of second polynucleotides, the plurality of third polynucleotides, and the second plurality of ligation templates to a third illumination, under conditions sufficient to break a subset of a plurality of second links generated in (e) between (i) the second subset of the plurality of second polynucleotides and the subset of the second plurality of ligation templates, and (ii) the subset of the plurality of third polynucleotides and the subset of the second plurality of ligation templates hybridized thereto.

In some embodiments, the second illumination comprises UV light. In some embodiments, the UV light comprises a wavelength of about 312 nanometers (nm).

In some embodiments, a plurality of analytes are immobilized to the plurality of individually addressable locations, and wherein the plurality of first polynucleotides are coupled to the plurality of analytes. In some embodiments, the method further comprises, prior to (a), (i) providing the substrate, (ii) immobilizing the plurality of analytes at the plurality of individually addressable locations, and (iii) coupling the plurality of first polynucleotides to the plurality of analytes.

In some embodiments, the plurality of first polynucleotides is coupled to the plurality of analytes via a plurality of analyte-binding moieties of the plurality of first polynucleotides.

In some embodiments, the plurality of analyte-binding moieties comprises comprise one or more members selected from the group consisting of: nucleic acids, proteins, lipids, saccharides, polysaccharides, and a combination thereof. In some embodiments, the proteins comprise antibodies or antigen binding fragments thereof.

In some embodiments, the plurality of analytes comprises one or more members selected from the group consisting of: nucleic acids, proteins, cells, lipids, saccharides, polysaccharides, and a combination thereof. In some embodiments, the proteins comprise one or more of extracellular proteins, cell surface proteins, cell membrane proteins, and intracellular proteins.

In some embodiments, a plurality of beads are immobilized to the plurality of individually addressable locations, and wherein the plurality of first polynucleotides are coupled to the plurality of beads.

In some embodiments, (c) comprises (i) cross-linking the subset of the plurality of first polynucleotides with the subset of the first plurality of ligation templates, and (ii) cross-linking the subset of the plurality of second polynucleotides with the subset of the first plurality of ligation templates.

In some embodiments, the plurality of first polynucleotides comprises a plurality of cross-linkers used in the cross-linking in (c)(i). In some embodiments, the plurality of cross-linkers comprises 3-Cyanovinylcarbazole nucleosides (CNVKs).

In some embodiments, the cross-linking in (c)(i) comprises forming a cross-link between a cross-linker of the plurality of cross-linkers and a nucleotide of the first plurality of ligation templates. In some embodiments, the nucleotide of the first plurality of ligation templates is a cytosine (C) or a thymine (T). In some embodiments, the nucleotide of the first plurality of ligation templates is a C. In some embodiments, the nucleotide of the first plurality of ligation templates is a T.

In some embodiments, (c) comprises (i) linking, via non-hydrogen bonds, the subset of the plurality of first polynucleotides with the subset of the first plurality of ligation templates, and (ii) linking, via non-hydrogen bonds, the subset of the plurality of second polynucleotides with the subset of the first plurality of ligation templates. In some embodiments, the non-hydrogen bonds comprise one or more of a covalent bond, cycloaddition bond, and photocycloaddition bond.

In some embodiments, the first fluid layer or the second fluid layer has a thickness of at most about 15 micrometers.

In another aspect, provided is a system for barcode generation, comprising: a substrate comprising a plurality of individually addressable locations; a plurality of first polynucleotides immobilized at the plurality of individually addressable locations, wherein the plurality of first polynucleotides comprises a plurality of first cross-linkers; and a fluid layer with a thickness of at most 50 micrometers on the substrate, wherein the fluid layer comprises: a plurality of second polynucleotides, wherein the plurality of second polynucleotides comprises a barcode sequence, wherein the plurality of second polynucleotides comprises a plurality of second cross-linkers; and a plurality of ligation templates, wherein each of the plurality of ligation templates comprises a first nucleotide configured to cross-link with the plurality of first cross-linkers and a second nucleotide configured to cross-link with the plurality of second cross-linkers.

In some embodiments, the system further comprises an illumination system, configured to selectively illuminate one or more subsets of individually addressable locations on the substrate. In some embodiments, the illumination system comprises a Digital Micromirror Device (DMD).

In some embodiments, at least a subset of the plurality of ligation templates are hybridized to a subset of the plurality of first polynucleotides.

In some embodiments, at least the subset of the plurality of ligation templates are hybridized to a subset of the plurality of second polynucleotides.

In some embodiments, a ligation template of the plurality of ligation templates is hybridized to (i) a first polynucleotide of the plurality of first polynucleotides, comprising a first cross-linker of the plurality of first cross-linkers, and (ii) a second polynucleotide of the plurality of second polynucleotides, comprising a second cross-linker of the plurality of second cross-linkers, and the first nucleotide of the ligation template is cross-linked with the first cross-linker.

In some embodiments, the second nucleotide of the ligation template is cross-linked with the second cross-linker.

In some embodiments, the system further comprises a plurality of analytes immobilized at the plurality of individually addressable locations, wherein the plurality of first polynucleotides are coupled to the plurality of analytes.

In some embodiments, the system further comprises a plurality of beads immobilized at the plurality of individually addressable locations, wherein the plurality of first polynucleotides are coupled to the plurality of beads.

In some embodiments, the fluid layer has a thickness of at most about 15 micrometers.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative instances of the present disclosure are shown and described. As will be realized, the present disclosure is capable of other and different instances, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein) of which:

FIG. 1 shows an example flowchart of a method for photolabile spatial barcoding.

FIG. 2 illustrates examples of individually addressable locations distributed on substrates, as described herein.

FIGS. 3A-3G illustrate different examples of cross-sectional surface profiles of a substrate, as described herein.

FIG. 4 shows an example coating of a substrate with a hexagonal lattice of beads, as described herein.

FIGS. 5A-5B illustrate example systems and methods for loading a sample or a reagent onto a substrate, as described herein.

FIG. 6 illustrates a computerized system for sequencing a nucleic acid molecule.

FIGS. 7A-7C illustrate multiplexed stations in a sequencing system.

FIG. 8 shows a computer system that is programmed or otherwise configured to implement methods provided herein.

FIG. 9A shows a schematic illustration of ultra-fast reversible photo-crosslinking of nucleic acids.

FIG. 9B shows efficacies of crosslinking reactions with different nucleotides at the indicated positions.

FIG. 10 shows a schematic representation of a Digital Micromirror Device (DMD) and DMD illuminated regions.

FIG. 11 shows a schematic illustration of differentially illuminated regions generated by two independent selective illumination cycles.

FIG. 12 shows example reagents for generating barcodes using photolabile cross-linking.

### DETAILED DESCRIPTION

While various instances of the invention have been shown and described herein, it will be obvious to those skilled in the art that such instances are provided by way of example only. Numerous variations, changes, and substitutions may occur to those skilled in the art without departing from the invention. It should be understood that various alternatives to the instances of the invention described herein may be employed.

Provided herein are systems, methods, kits, and compositions for generating spatial barcodes at distinct spatial locations of a substrate, directed by iterative cycles of selective illumination on the substrate. In some cases, the spatial barcodes may be generated on a surface of the substrate at such spatial locations. In other cases, the spatial locations on the substrate may have immobilized thereto one or more analytes, and the spatial barcodes may be generated directly on the one or more analytes at such spatial locations. The identity and location of a spatial barcode generated on the substrate can be determined by tracing (i) the known identity of the sets of unique sequences added and (ii) the location of selective illumination, during each cycle of selective illumination.

As used herein, the singular forms “a,” “an,” and “the” include the plural reference unless the context clearly dictates otherwise.

When a range of values is provided, it is to be understood that each intervening value between the upper and lower limit of that range, and any other stated or intervening value in that stated range is encompassed within the scope of the present disclosure. Where the stated range includes upper or lower limits, ranges excluding either of those included limits are also included in the present disclosure.

### Definitions

The term “biological sample,” as used herein, generally refers to any sample derived from a subject or specimen. The biological sample can be a fluid, tissue, collection of cells (e.g., check swab), hair sample, or feces sample. The fluid can be blood (e.g., whole blood), saliva, urine, or sweat. The tissue can be from an organ (e.g., liver, lung, or thyroid), or a mass of cellular material, such as, for example, a tumor. The biological sample can be a cellular sample or cell-free sample. Examples of biological samples include nucleic acid molecules, amino acids, polypeptides, proteins, carbohydrates, fats, or viruses. In an example, a biological sample is a nucleic acid sample including one or more nucleic acid molecules, such as deoxyribonucleic acid (DNA) and/or ribonucleic acid (RNA). The nucleic acid sample may comprise cell-free nucleic acid molecules, such as cell-free DNA or cell-free RNA. Further, samples may be extracted from variety of animal fluids containing cell free sequences, including but not limited to blood, serum, plasma, vitreous, sputum, urine, tears, perspiration, saliva, semen, mucosal excretions, mucus, spinal fluid, amniotic fluid, lymph fluid and the like. Cell free polynucleotides may be fetal in origin (via fluid taken from a pregnant subject) or may be derived from tissue of the subject itself. A biological sample may also refer to a sample engineered to mimic one or more properties (e.g., nucleic acid sequence properties, e.g., sequence identity, length, GC content, etc.) of a native sample derived from a subject or specimen.

The term “subject,” as used herein, generally refers to an individual from whom a biological sample is obtained. The subject may be a mammal or non-mammal. The subject may be human, non-human mammal, animal, ape, monkey, chimpanzee, reptilian, amphibian, avian, or a plant. The subject may be a patient. The subject may be displaying a symptom of a disease. The subject may be asymptomatic. The subject may be undergoing treatment. The subject may not be undergoing treatment. The subject can have or be suspected of having a disease, such as cancer (e.g., breast cancer, colorectal cancer, brain cancer, leukemia, lung cancer, skin cancer, liver cancer, pancreatic cancer, lymphoma, esophageal cancer, cervical cancer, etc.) or an infectious disease. The subject can have or be suspected of having a genetic disorder such as achondroplasia, alpha-1 antitrypsin deficiency, antiphospholipid syndrome, autism, autosomal dominant polycystic kidney disease, Charcot-Marie-tooth, cri du chat, Crohn's disease, cystic fibrosis, Dercum disease, down syndrome, Duane syndrome, Duchenne muscular dystrophy, factor V Leiden thrombophilia, familial hypercholesterolemia, familial Mediterranean fever, fragile x syndrome, Gaucher disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency, sickle cell disease, spinal muscular atrophy, Tay-Sachs, thalassemia, trimethylaminuria, Turner syndrome, velocardiofacial syndrome, WAGR syndrome, or Wilson disease.

The term “analyte,” as used herein, generally refers to an object that is the subject of analysis, or an object, regardless of being the subject of analysis, that is directly or indirectly analyzed during a process. An analyte may be synthetic. An analyte may be, originate from, and/or be derived from, a sample, such as a biological sample. In some examples, an analyte is or includes a molecule, macromolecule (e.g., nucleic acid, carbohydrate, protein, lipid, etc.), nucleic acid, carbohydrate, lipid, antibody, antibody fragment, antigen, peptide, polypeptide, protein, macromolecular group (e.g., glycoproteins, proteoglycans, ribozymes, liposomes, etc.), cell, tissue, biological particle, or an organism, or any engineered copy or variant thereof, or any combination thereof. The term “processing an analyte,” as used herein, generally refers to one or more stages of interaction with one more samples. Processing an analyte may comprise conducting a chemical reaction, biochemical reaction, enzymatic reaction, hybridization reaction, polymerization reaction, physical reaction, any other reaction, or a combination thereof with, in the presence of, or on, the analyte. Processing an analyte may comprise physical and/or chemical manipulation of the analyte. For example, processing an analyte may comprise detection of a chemical change or physical change, addition of or subtraction of material, atoms, or molecules, molecular confirmation, detection of the presence of a fluorescent label, detection of a Forster resonance energy transfer (FRET) interaction, or inference of absence of fluorescence.

The terms “nucleic acid,” “nucleic acid molecule,” “nucleic acid sequence,” “nucleic acid fragment,” “oligonucleotide” and “polynucleotide,” as used herein, generally refer to a polynucleotide that may have various lengths of bases, comprising, for example, deoxyribonucleotide, deoxyribonucleic acid (DNA), ribonucleotide, or ribonucleic acid (RNA), or analogs thereof. A nucleic acid may be single-stranded. A nucleic acid may be double-stranded. A nucleic acid may be partially double-stranded, such as to have at least one double-stranded region and at least one single-stranded region. A partially double-stranded nucleic acid may have one or more overhanging regions. An “overhang,” as used herein, generally refers to a single-stranded portion of a nucleic acid that extends from or is contiguous with a double-stranded portion of a same nucleic acid molecule. Non-limiting examples of nucleic acids include DNA, RNA, genomic DNA or synthetic DNA/RNA or coding or non-coding regions of a gene or gene fragment, loci (locus) defined from linkage analysis, exons, introns, messenger RNA (mRNA), transfer RNA, ribosomal RNA (rRNA), short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, and isolated RNA of any sequence. A nucleic acid can have a length of at least about 10 nucleic acid bases (“bases”), 20 bases, 30 bases, 40 bases, 50 bases, 100 bases, 200 bases, 300 bases, 400 bases, 500 bases, 1 kilobase (kb), 2 kb, 3, kb, 4 kb, 5 kb, 10 kb, 20 kb, 30 kb, 40 kb, 50 kb, 100 kb, 200 kb, 300 kb, 400 kb, 500 kb, 1 megabase (Mb), 10 Mb, 100 Mb, 1 gigabase or more. A nucleic acid may comprise A nucleic acid can comprise a sequence of four natural nucleotide bases: adenine (A); cytosine (C); guanine (G); and thymine (T) (uracil (U) for thymine (T) when the nucleic acid is RNA). A nucleic acid may include one or more nonstandard nucleotide(s), nucleotide analog(s) and/or modified nucleotide(s).

The term “nucleotide,” as used herein, generally refers to any nucleotide or nucleotide analog. The nucleotide may be naturally occurring or non-naturally occurring. The nucleotide may be a modified, synthesized, or engineered nucleotide. The nucleotide may include a canonical base or a non-canonical base. The nucleotide may comprise an alternative base. The nucleotide may include a modified polyphosphate chain (e.g., triphosphate coupled to a fluorophore). The nucleotide may comprise a label. The nucleotide may be terminated (e.g., reversibly terminated). Nonstandard nucleotides, nucleotide analogs, and/or modified analogs may include, but are not limited to, diaminopurine, 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid(v), 5-methyl-2-thiouracil, 3-(3-amino-3-N-2-carboxypropyl) uracil, (acp3)w, 2,6-diaminopurine, ethynyl nucleotide bases, 1-propynyl nucleotide bases, azido nucleotide bases, phosphoroselenoate nucleic acids and the like. In some cases, nucleotides may include modifications in their phosphate moieties, including modifications to a triphosphate moiety. Additional, non-limiting examples of modifications include phosphate chains of greater length (e.g., a phosphate chain having, 4, 5, 6, 7, 8, 9, 10 or more phosphate moieties), modifications with thiol moieties (e.g., alpha-thiol triphosphate and beta-thiotriphosphates) or modifications with selenium moieties (e.g., phosphoroselenoate nucleic acids). Nucleic acids may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. Nucleic acids may also contain amine-modified groups, such as aminoallyl-dUTP (aa-dUTP) and aminohexhylacrylamide-dCTP (aha-dCTP) to allow covalent attachment of amine reactive moieties, such as N-hydroxysuccinimide esters (NHS). Alternatives to standard DNA base pairs or RNA base pairs in the oligonucleotides of the present disclosure can provide higher density in bits per cubic mm, higher safety (resistant to accidental or purposeful synthesis of natural toxins), easier discrimination in photo-programmed polymerases, or lower secondary structure. Nucleotides may be capable of reacting or bonding with detectable moieties for nucleotide detection.

The term “sequencing,” as used herein, generally refers to a process for generating or identifying a sequence of a biological molecule, such as a nucleic acid. The sequence may be a nucleic acid sequence which comprises a sequence of nucleic acid bases. As used herein, the term “template nucleic acid” generally refers to the nucleic acid to be sequenced. The template nucleic acid may be an analyte or be associated with an analyte. For example, the analyte can be a mRNA, and the template nucleic acid is the mRNA, or a cDNA derived from the mRNA, or another derivative thereof. In another example, the analyte can be a protein, and the template nucleic acid is an oligonucleotide that is conjugated to an antibody that binds to the protein, or derivative thereof. Sequencing may be single molecule sequencing or sequencing by synthesis, for example. Sequencing may comprise generating sequencing signals and/or sequencing reads. Sequencing may be performed on template nucleic acids immobilized on a support, such as a flow cell, substrate, and/or one or more beads. In some cases, a template nucleic acid may be amplified to produce a colony of nucleic acid molecules attached to the support to produce amplified sequencing signals. In one example, (i) a template nucleic acid is subjected to a nucleic acid reaction, e.g., amplification, to produce a clonal population of the nucleic acid attached to a bead, the bead immobilized to a substrate, (ii) amplified sequencing signals from the immobilized bead are detected from the substrate surface during or following one or more nucleotide flows, and (iii) the sequencing signals are processed to generate sequencing reads. The substrate surface may immobilize multiple beads at distinct locations, each bead containing distinct colonies of nucleic acids, and upon detecting the substrate surface, multiple sequencing signals may be simultaneously or substantially simultaneously processed from the different immobilized beads at the distinct locations to generate multiple sequencing reads. In some sequencing methods, the nucleotide flows comprise non-terminated nucleotides. In some sequencing methods, the nucleotide flows comprise terminated nucleotides.

The terms “amplifying,” “amplification,” and “nucleic acid amplification” are used interchangeably and generally refer to generating one or more copies of a nucleic acid or a template. For example, “amplification” of DNA generally refers to generating one or more copies of a DNA molecule. Amplification of a nucleic acid may be linear, exponential, or a combination thereof. Amplification may be emulsion based or non-emulsion based. Non-limiting examples of nucleic acid amplification methods include reverse transcription, primer extension, polymerase chain reaction (PCR), ligase chain reaction (LCR), helicase-dependent amplification, asymmetric amplification, rolling circle amplification (RCA), recombinase polymerase reaction (RPA), loop mediated isothermal amplification (LAMP), nucleic acid sequence based amplification (NASBA), self-sustained sequence replication (3SR), and multiple displacement amplification (MDA). Where PCR is used, any form of PCR may be used, with non-limiting examples that include real-time PCR, allele-specific PCR, assembly PCR, asymmetric PCR, digital PCR, emulsion PCR (ePCR or emPCR), dial-out PCR, helicase-dependent PCR, nested PCR, hot start PCR, inverse PCR, methylation-specific PCR, miniprimer PCR, multiplex PCR, nested PCR, overlap-extension PCR, thermal asymmetric interlaced PCR, and touchdown PCR. Amplification can be conducted in a reaction mixture comprising various components (e.g., a primer(s), template, nucleotides, a polymerase, buffer components, co-factors, etc.) that participate or facilitate amplification. In some cases, the reaction mixture comprises a buffer that permits context independent incorporation of nucleotides. Non-limiting examples include magnesium-ion, manganese-ion and isocitrate buffers. Additional examples of such buffers are described in Tabor, S. et al. C. C. PNAS, 1989, 86, 4076-4080 and U.S. Pat. Nos. 5,409,811 and 5,674,716, each of which is herein incorporated by reference in its entirety. Useful methods for clonal amplification from single molecules include rolling circle amplification (RCA) (Lizardi et al., Nat. Genet. 19:225-232 (1998), which is incorporated herein by reference), bridge PCR (Adams and Kron, Method for Performing Amplification of Nucleic Acid with Two Primers Bound to a Single Solid Support, Mosaic Technologies, Inc. (Winter Hill, Mass.); Whitehead Institute for Biomedical Research, Cambridge, Mass., (1997); Adessi et al., Nucl. Acids Res. 28:E87 (2000); Pemov et al., Nucl. Acids Res. 33:e11(2005); or U.S. Pat. No. 5,641,658, each of which is incorporated herein by reference), polony generation (Mitra et al., Proc. Natl. Acad. Sci. USA 100:5926-5931 (2003); Mitra et al., Anal. Biochem. 320:55-65(2003), each of which is incorporated herein by reference), and clonal amplification on beads using emulsions (Dressman et al., Proc. Natl. Acad. Sci. USA 100:8817-8822 (2003), which is incorporated herein by reference) or ligation to bead-based adapter libraries (Brenner et al., Nat. Biotechnol. 18:630-634 (2000); Brenner et al., Proc. Natl. Acad. Sci. USA 97:1665-1670 (2000)); Reinartz, et al., Brief Funct. Genomic Proteomic 1:95-104 (2002), each of which is incorporated herein by reference). Amplification products from a nucleic acid may be identical or substantially identical. A nucleic acid colony resulting from amplification may have identical or substantially identical sequences.

As used herein, the terms “identical” or “percent identity,” when used with respect to two or more nucleic acid or polypeptide sequences, refer to two or more sequences that are the same or, alternatively, have a specified percentage of amino acid residues or nucleotides that are the same, when compared and aligned for maximum correspondence, as measured using any one or more of the following sequence comparison algorithms: Needleman-Wunsch (see, e.g., Needleman, Saul B.; and Wunsch, Christian D. (1970). “A general method applicable to the search for similarities in the amino acid sequence of two proteins” Journal of Molecular Biology 48 (3):443-53); Smith-Waterman (see, e.g., Smith, Temple F.; and Waterman, Michael S., “Identification of Common Molecular Subsequences” (1981) Journal of Molecular Biology 147:195-197); or BLAST (Basic Local Alignment Search Tool; sec, e.g., Altschul S F, Gish W, Miller W, Myers E W, Lipman D J, “Basic local alignment search tool” (1990) J Mol Biol 215 (3):403-410). As used herein, the terms “substantially identical” or “substantial identity” when used with respect to two or more nucleic acid or polypeptide sequences, refer to two or more sequences or subsequences (such as biologically active fragments) that have at least 60%, at least 70%, at least 75%, at least 80%, at least 85%, at least 90%, at least 95%, at least 96%, at least 97%, at least 98%, or at least 99% nucleotide or amino acid residue identity, when compared and aligned for maximum correspondence, as measured using a sequence comparison algorithm or by visual inspection. Substantially identical sequences are typically considered to be homologous without reference to actual ancestry. In some embodiments, “substantial identity” exists over a region of the sequences being compared. In some embodiments, substantial identity exists over a region of at least 25 residues in length, at least 50 residues in length, at least 100 residues in length, at least 150 residues in length, at least 200 residues in length, or greater than 200 residues in length. In some embodiments, the sequences being compared are substantially identical over the full length of the sequences being compared. Typically, substantially identical nucleic acid or protein sequences include less than 100% nucleotide or amino acid residue identity as such sequences would generally be considered “identical.”

The term “coupled to,” as used herein, generally refers to an association between two or more objects that may be temporary or substantially permanent. A first object may be reversibly or irreversibly coupled to a second object. For example, a nucleic acid molecule may be reversibly coupled to a particle. A reversible coupling may comprise, for example, a releasable coupling (e.g., in which a first object may be released from a second object to which it is coupled). A first object reversibly coupled (or releasably coupled) to a second object may be separated from the second object, e.g., upon application of a stimulus, which stimulus may comprise a photostimulus (e.g., ultraviolet light), a thermal stimulus, a chemical stimulus (e.g., reducing agent), or any other useful stimulus. Coupling may encompass immobilization to a support (e.g., as described herein). Similarly, coupling may encompass attachment, such as attachment of a first object to a second object. Coupling may comprise any interaction that affects an association between two objects, including, for example, a covalent bond, a non-covalent interaction (e.g., electrostatic interaction [e.g., hydrogen bonding, ionic interaction, and halogen bonding], π-interaction [e.g., π-π interaction, polar-π interaction, cation-π interaction, and anion-π interaction], van der Waals force-based interactions [e.g., dipole-dipole interactions, dipole-induced dipole interactions, and induced dipole-induced dipole interactions], hydrophobic interaction), a magnetic interaction (e.g., magnetic dipole-dipole interaction, indirect dipole-dipole coupling), an electromagnetic interaction, adsorption, or any other useful interaction. For example, a particle may be coupled to a planar support via an electrostatic interaction, a magnetic interaction, or a covalent interaction. Similarly, a nucleic acid molecule may be coupled to a particle via a covalent interaction or via a non-covalent interaction. A coupling between a first object and a second object may comprise a labile moiety, such as a moiety comprising an ester, vicinal diol, phosphodiester, peptide, glycosidic, sulfone, Diels-Alder, or similar linkage. The strength of a coupling between a first object and a second object may be indicated by a dissociation constant, K_{d}, that indicates the inclination of a coupled object comprising a first object and a second object to dissociate into the uncoupled first and second objects and may be expressed as a ratio of dissociated (e.g., uncoupled) objects to coupled objects.

### Open Substrate Systems

Described herein are devices, systems, and methods that use open substrates or open flow cell geometries to process a sample. The term “open substrate,” as used herein, generally refers to a substrate in which any point on an active surface of the substrate is physically accessible from a direction normal to the substrate. The devices, systems and methods may be used to facilitate any application or process involving a reaction or interaction between two objects, such as between an analyte and a reagent or between two reagents. For example, the reaction or interaction may be chemical (e.g., polymerase reaction) or physical (e.g., displacement). The devices, systems, and methods described herein may benefit from higher efficiency, such as from faster reagent delivery and lower volumes of reagents required per surface area. The devices, systems, and methods described herein may avoid contamination problems common to microfluidic channel flow cells that are fed from multiport valves which can be a source of carryover from one reagent to the next. The devices, systems, and methods may benefit from shorter completion time, use of fewer resources (e.g., various reagents), and/or reduced system costs. The open substrates or flow cell geometries may be used to process any analyte from any sample, such as but not limited to, nucleic acid molecules, protein molecules, antibodies, antigens, cells, and/or organisms, as described herein. The open substrates or flow cell geometries may be used for any application or process, such as, but not limited to, sequencing by synthesis, sequencing by ligation, amplification, proteomics, single cell processing, barcoding, and sample preparation, as described herein.

A sample processing system may comprise a substrate, and devices and systems that perform one or more operations with or on the substrate. The sample processing system may permit highly efficient dispensing of reagents onto the substrate. The sample processing may permit highly efficient imaging of one or more analytes, or signals corresponding thereto, on the substrate. The sample processing system may comprise an imaging system comprising a detector. Substrates and detectors that can be used in the sample processing system are described in further detail in U.S. Patent Pub Nos. 2020/0326327, 2021/0354126, and 2021/0079464, each of which is entirely incorporated herein by reference for all purposes.

### Substrates

The substrate may be a solid substrate. The substrate may entirely or partially comprise one or more of rubber, glass, silicon, a metal such as aluminum, copper, titanium, chromium, or steel, a ceramic such as titanium oxide or silicon nitride, a plastic such as polyethylene (PE), low-density polyethylene (LDPE), high-density polyethylene (HDPE), polypropylene (PP), polystyrene (PS), high impact polystyrene (HIPS), polyvinyl chloride (PVC), polyvinylidene chloride (PVDC), acrylonitrile butadiene styrene (ABS), polyacetylene, polyamides, polycarbonates, polyesters, polyurethanes, polyepoxide, polymethyl methacrylate (PMMA), polytetrafluoroethylene (PTFE), phenol formaldehyde (PF), melamine formaldehyde (MF), urea-formaldehyde (UF), polyetheretherketone (PEEK), polyetherimide (PEI), polyimides, polylactic acid (PLA), furans, silicones, polysulfones, any mixture of any of the preceding materials, or any other appropriate material. The substrate may be entirely or partially coated with one or more layers of a metal such as aluminum, copper, silver, or gold, an oxide such as a silicon oxide (SiₓO_{y}, where x, y may take on any possible values), a photoresist such as SU8, a surface coating such as an aminosilane or hydrogel, polyacrylic acid, polyacrylamide dextran, polyethylene glycol (PEG), or any combination of any of the preceding materials, or any other appropriate coating. The substrate may comprise multiple layers of the same or different type of material. The substrate may be fully or partially opaque to visible light. The substrate may be fully or partially transparent to visible light. A surface of the substrate may be modified to comprise active chemical groups, such as amines, esters, hydroxyls, epoxides, and the like, or a combination thereof. A surface of the substrate may be modified to comprise any of the binders or linkers described herein. In some instances, such binders, linkers, active chemical groups, and the like may be added as an additional layer or coating to the substrate.

The substrate may have the general form of a cylinder, a cylindrical shell or disk, a rectangular prism, or any other geometric form. The substrate may have a thickness (e.g., a minimum dimension) of at least 100 micrometers (μm), at least 200 μm, at least 500 μm, at least 1 millimeter (mm), at least 2 mm, at least 5 mm, at least 10 mm, or more. The substrate may have a first lateral dimension (such as a width for a substrate having the general form of a rectangular prism or a radius or diameter for a substrate having the general form of a cylinder) and/or a second lateral dimension (such as a length for a substrate having the general form of a rectangular prism) of at least 1 mm, at least 2 mm, at least 5 mm, at least 10 mm, at least 20 mm, at least 50 mm, at least 100 mm, at least 200 mm, at least 500 mm, at least 1,000 mm, or more.

One or more surfaces of the substrate may be exposed to a surrounding open environment, and accessible from such surrounding open environment. For example, the array may be exposed and accessible from such surrounding open environment. In some cases, as described elsewhere herein, the surrounding open environment may be controlled and/or confined in a larger controlled environment.

The substrate may comprise a plurality of individually addressable locations. The individually addressable locations may comprise locations that are physically accessible for manipulation. The manipulation may comprise, for example, placement, extraction, reagent dispensing, seeding, heating, cooling, or agitation. The manipulation may be accomplished through, for example, localized microfluidic, pipet, optical, laser, acoustic, magnetic, and/or electromagnetic interactions with the analyte or its surroundings. The individually addressable locations may comprise locations that are digitally accessible. For example, each individually addressable location may be located, identified, and/or accessed electronically or digitally for indexing, mapping, sensing, associating with a device (e.g., detector, processor, dispenser, etc.), or otherwise processing.

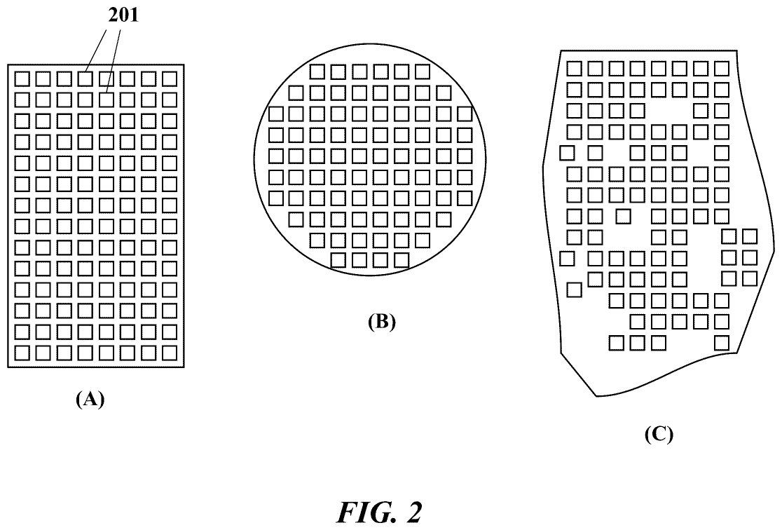

The plurality of individually addressable locations may be arranged as an array, randomly, or according to any pattern, on the substrate. FIG. 2 illustrates different substrates (from a top view) comprising different arrangements of individually addressable locations 201, with panel A showing a substantially rectangular substrate with regular linear arrays, panel B showing a substantially circular substrate with regular linear arrays, and panel C showing an arbitrarily shaped substrate with irregular arrays. The substrate may have any number of individually addressable locations, for example, at least 1, at least 2, at least 5, at least 10, at least 20, at least 50, at least 100, at least 200, at least 500, at least 1,000, at least 2,000, at least 5,000, at least 10,000, at least 20,000, at least 50,000, at least 100,000, at least 200,000, at least 500,000, at least 1,000,000, at least 2,000,000, at least 5,000,000, at least 10,000,000, at least 20,000,000, at least 50,000,000, at least 100,000,000, at least 200,000,000, at least 500,000,000, at least 1,000,000,000, at least 2,000,000,000, at least 5,000,000,000, at least 10,000,000,000, at least 20,000,000,000, at least 50,000,000,000, at least 100,000,000,000 or more individually addressable locations. The substrate may have a number of individually addressable locations that is within a range defined by any two of the preceding values.

Each individually addressable location may have the general shape or form of a circle, pit, bump, rectangle, or any other shape or form (e.g., polygonal, non-polygonal). A plurality of individually addressable locations can have uniform shape or form, or different shapes or forms. An individually addressable location may have any size. In some cases, an individually addressable location may have an area of about 0.1 square micron (μm²), about 0.2 μm², about 0.25 μm², about 0.3 μm², about 0.4 μm², about 0.5 μm², about 0.6 μm², about 0.7 μm², about 0.8 μm², about 0.9 μm², about 1 μm², about 1.1 μm², about 1.2 μm², about 1.25 μm², about 1.3 μm², about 1.4 μm², about 1.5 μm², about 1.6 μm², about 1.7 μm², about 1.75 μm², about 1.8 μm², about 1.9 μm², about 2 μm², about 2.25 μm², about 2.5 μm², about 2.75 μm², about 3 μm², about 3.25 μm², about 3.5 μm², about 3.75 μm², about 4 μm², about 4.25 μm², about 4.5 μm², about 4.75 μm², about 5 μm², about 5.5 μm², about 6 μm², or more. An individually addressable location may have an area that is within a range defined by any two of the preceding values. An individually addressable location may have an area that is less than about 0.1 μm² or greater than about 6 μm².

The individually addressable locations may be distributed on a substrate with a pitch determined by the distance between the center of a first location and the center of the closest or neighboring individually addressable location. Locations may be spaced with a pitch of about 0.1 micron (μm, ), about 0.2 μm, about 0.25 μm, about 0.3 μm, about 0.4 μm, about 0.5 μm, about 0.6 μm, about 0.7 μm, about 0.8 μm, about 0.9 μm, about 1 μm, about 1.1 μm, about 1.2 μm, about 1.25 μm, about 1.3 μm, about 1.4 μm, about 1.5 μm, about 1.6 μm, about 1.7 μm, about 1.75 μm, about 1.8 μm, about 1.9 μm, about 2 μm, about 2.25 μm, about 2.5 μm, about 2.75 μm, about 3 μm, about 3.25 μm, about 3.5 μm, about 3.75 μm, about 4 μm, about 4.25 μm, about 4.5 μm, about 4.75 μm, about 5 μm, about 5.5 μm, about 6 μm, about 6.5 μm, about 7 μm, about 7.5 μm, about 8 μm, about 8.5 μm, about 9 μm, about 9.5 μm, or about 10 μm. In some cases, the locations may be positioned with a pitch that is within a range defined by any two of the preceding values. The locations may be positioned with a pitch of less than about 0.1 μm, or greater than about 10 μm. In some cases, the pitch between two individually addressable locations may be determined as a function of a size of a loading object (e.g., bead). For example, where the loading object is a bead having a maximum diameter, the pitch may be at least about the maximum diameter of the loading object.

Each of the plurality of individually addressable locations, or each of a subset of such locations, may be capable of immobilizing thereto an analyte (e.g., a nucleic acid molecule, a protein molecule, a carbohydrate molecule, etc.) or a reagent (e.g., a nucleic acid molecule, a probe molecule, a barcode molecule, an antibody molecule, a primer molecule, a bead, etc.). In some cases, an analyte or reagent may be immobilized to an individually addressable location via a support, such as a bead. In an example, a bead is immobilized to the individually addressable location, and the analyte or reagent is immobilized to the bead. In some cases, an individually addressable location may immobilize thereto a plurality of analytes or a plurality of reagents, such as via the support. The substrate may immobilize a plurality of analytes or reagents across multiple individually addressable locations. The plurality of analytes or reagents may be of the same type of analyte or reagent (e.g., a nucleic acid molecule) or may be a combination of different types of analytes or reagents (e.g., nucleic acid molecules, protein molecules, etc.). In an example, a first bead comprising a first colony of nucleic acid molecules each comprising a first template sequence is immobilized to a first individually addressable location, and a second bead comprising a second colony of nucleic acid molecules each comprising a second template sequence is immobilized to a second individually addressable location.

A substrate may comprise more than one type of individually addressable location arranged as an array, randomly, or according to any pattern, on the substrate. In some cases, different types of individually addressable locations may have different chemical, physical, and/or biological properties (e.g., hydrophobicity, charge, color, topography, size, dimensions, geometry, etc.). For example, a first type of individually addressable location may bind a first type of biological analyte but not a second type of biological analyte, and a second type of individually addressable location may bind the second type of biological analyte but not the first type of biological analyte.

In some cases, an individually addressable location may comprise a distinct surface chemistry. The distinct surface chemistry may distinguish between different addressable locations. The distinct surface chemistry may distinguish an individually addressable location from a surrounding location on the substrate. For example, a first location type may comprise a first surface chemistry, and a second location type may lack the first surface chemistry. In another example, the first location type may comprise the first surface chemistry and the second location type may comprise a second, different surface chemistry. A first location type may have a first affinity towards an object (e.g., a bead comprising nucleic acid molecules, e.g., amplicons, immobilized thereto) and a second location type may have a second, different affinity towards the same object due to different surface chemistries. In other examples, a first location type comprising a first surface chemistry may have an affinity towards a first sample type (e.g., a bead comprising nucleic acid molecules, e.g., amplicons, immobilized thereto) and exclude a second sample type (e.g., a bead lacking nucleic acid molecules, e.g., amplicons, immobilized thereto). The first location type and the second location type may or may not be disposed on the surface in alternating fashion. For example, a first location type or region type may comprise a positively charged surface chemistry and a second location type or region type may comprise a negatively charged surface chemistry. In another example, a first location type or region type may comprise a hydrophobic surface chemistry and a second location type or region type may comprise a hydrophilic surface chemistry. In another example, a first location type comprises a binder, as described elsewhere herein, and a second location type does not comprise the binder or comprises a different binder. In some cases, a surface chemistry may comprise an amine. In some cases, a surface chemistry may comprise a silane (e.g., tetramethylsilane). In some cases, the surface chemistry may comprise hexamethyldisilazane (HMDS). In some cases, the surface chemistry may comprise (3-aminopropyl)triethoxysilane (APTMS). In some cases, the surface chemistry may comprise a surface primer molecule or any oligonucleotide molecule that has any degree of affinity towards another molecule. In one example, the substrate comprises a plurality of individually addressable locations, each defined by APTMS, which are positively charged and has affinity towards an amplified bead (e.g., a bead comprising nucleic acid molecules, e.g., amplicons, immobilized thereto) which exhibits a negative charge. The locations surrounding the plurality of individually addressable locations may comprise HMDS which repels amplified beads.

In some cases, the individually addressable locations may be indexed, e.g., spatially. Data corresponding to an indexed location, collected over multiple periods of time, may be linked to the same indexed location. In some cases, sequencing signal data collected from an indexed location, during iterations of sequencing-by-synthesis flows, are linked to the indexed location to generate a sequencing read for an analyte immobilized at the indexed location. In some embodiments, the individually addressable locations are indexed by demarcating part of the surface, such as by etching or notching the surface, using a dye or ink, depositing a topographical mark, depositing a sample (e.g., a control nucleic acid sample), depositing a reference object (e.g., e.g., a reference bead that always emits a detectable signal during detection), and the like, and the individually addressable locations may be indexed with reference to such demarcations. As will be appreciated, a combination of positive demarcations and negative demarcations (lack thereof) may be used to index the individually addressable locations. In some embodiments, each of the individually addressable locations is indexed. In some embodiments, a subset of the individually addressable locations is indexed. In some embodiments, the individually addressable locations are not indexed, and a different region of the substrate is indexed.

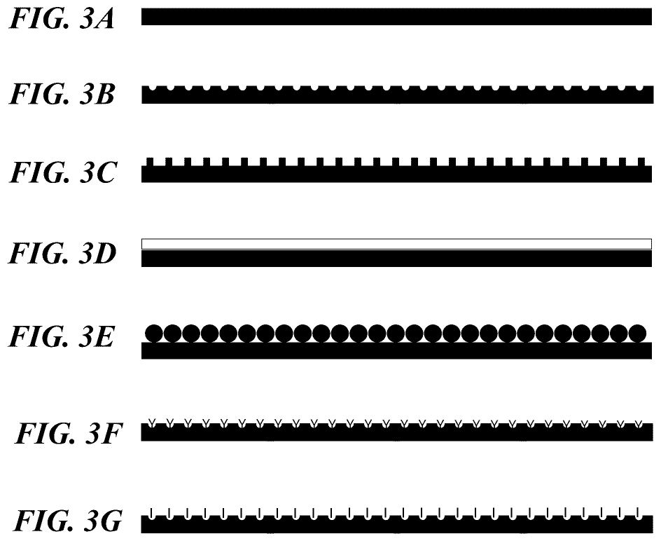

The substrate may comprise a planar or substantially planar surface. Substantially planar may refer to planarity at a micrometer level (e.g., a range of unevenness on the planar surface does not exceed the micrometer scale) or nanometer level (e.g., a range of unevenness on the planar surface does not exceed the nanometer scale). Alternatively, substantially planar may refer to planarity at less than a nanometer level or greater than a micrometer level (e.g., millimeter level). Alternatively or in addition, a surface of the substrate may be textured or patterned. For example, the substrate may comprise grooves, troughs, hills, and/or pillars. The substrate may define one or more cavities (e.g., micro-scale cavities or nano-scale cavities). The substrate may define one or more channels. The substrate may have regular textures and/or patterns across the surface of the substrate. For example, the substrate may have regular geometric structures (e.g., wedges, cuboids, cylinders, spheroids, hemispheres, etc.) above or below a reference level of the surface. Alternatively, the substrate may have irregular textures and/or patterns across the surface of the substrate. In some instances, a texture of the substrate may comprise structures having a maximum dimension of at most about 100%, 90%, 80%, 70%, 60%, 50%, 40%, 30%, 20%, 10%, 9%, 8%, 7%, 6%, 5%, 4%, 3%, 2%, 1%, 0.1%, 0.01%, 0.001%, 0.0001%, 0.00001% of the total thickness of the substrate or a layer of the substrate. In some instances, the textures and/or patterns of the substrate may define at least part of an individually addressable location on the substrate. A textured and/or patterned substrate may be substantially planar. FIGS. 3A-3G illustrate different examples of cross-sectional surface profiles of a substrate. FIG. 3A illustrates a cross-sectional surface profile of a substrate having a completely planar surface. FIG. 3B illustrates a cross-sectional surface profile of a substrate having semi-spherical troughs or grooves. FIG. 3C illustrates a cross-sectional surface profile of a substrate having pillars, or alternatively or in conjunction, wells. FIG. 3D illustrates a cross-sectional surface profile of a substrate having a coating. FIG. 3E illustrates a cross-sectional surface profile of a substrate having spherical particles. FIG. 3F illustrates a cross-sectional surface profile of FIG. 3B, with a first type of binders seeded or associated with the respective grooves. FIG. 3G illustrates a cross-sectional surface profile of FIG. 3B, with a second type of binders seeded or associated with the respective grooves.

A binder may be configured to immobilize an analyte or reagent to an individually addressable location. In some cases, a surface chemistry of an individually addressable location may comprise one or more binders. In some cases, a plurality of individually addressable locations may be coated with binders. In some cases, at least 10%, at least 20%, at least 30%, at least 40%, at least 50%, at least 60%, at least 70%, at least 80%, at least 90%, at least 91%, at least 92%, at least 93%, at least 94%, at least 95%, at least 96%, at least 97%, at least 98%, or at least 99% of the total number of individually addressable locations, or of the surface area of the substrate, are coated with binders. The binders may be integral to the array. The binders may be added to the array. For instance, the binders may be added to the array as one or more coating layers on the array. The substrate may comprise an order of magnitude of at least about 10, 100, 10³, 10⁴, 10⁵, 10⁶, 10⁷, 10⁸, 10⁹, 10¹⁰, 10¹¹, or more binders. Alternatively or in addition, the substrate may comprise an order of magnitude of at most about 10¹¹, 10¹⁰, 10⁹, 10⁸, 10⁷, 10⁶, 10⁵, 10⁴, 10³, 100, 10 or fewer binders.

The binders may immobilize analytes or reagents through non-specific interactions, such as one or more of hydrophilic interactions, hydrophobic interactions, electrostatic interactions, physical interactions (for instance, adhesion to pillars or settling within wells), and the like. Alternatively or in addition, the binders may immobilize analytes or reagents through specific interactions. For instance, where the analyte or reagent is a nucleic acid molecule, the binders may comprise oligonucleotide adaptors configured to bind to the nucleic acid molecule. In other examples, the binders may comprise one or more of antibodies or antigen binding fragments thereof, oligonucleotides, nucleic acid molecules, aptamers, affinity binding proteins, lipids, carbohydrates, and the like. The binders may immobilize analytes or reagents through any possible combination of interactions. For instance, the binders may immobilize nucleic acid molecules through a combination of physical and chemical interactions, through a combination of protein and nucleic acid interactions, etc. In some instances, a single binder may bind a single analyte (e.g., nucleic acid molecule) or single reagent. In some instances, a single binder may bind a plurality of analytes (e.g., plurality of nucleic acid molecules) or a plurality of reagents. In some instances, a plurality of binders may bind a single analyte or a single reagent. Though examples herein describe interactions of binders with nucleic acid molecules, the binders may immobilize other molecules (such as proteins), other particles, cells, viruses, other organisms, or the like. Though examples herein describe interactions of binders with samples or analytes, the binders may similarly immobilize reagents. In some instances, the substrate may comprise a plurality of types of binders, for example to bind different types of analytes or reagents. For example, a first type of binders (e.g., oligonucleotides) are configured to bind a first type of analyte (e.g., nucleic acid molecules) or reagent, and a second type of binders (e.g., antibodies) are configured to bind a second type of analyte (e.g., proteins) or reagent. In another example, a first type of binders (e.g., first type of oligonucleotide molecules) are configured to bind a first type of nucleic acid molecules and a second type of binders (e.g., second type of oligonucleotide molecules) are configured to bind a second type of nucleic acid molecules. For example, the substrate may be configured to bind different types of analytes or reagents in certain fractions or specific locations on the substrate by having the different types of binders in the certain fractions or specific locations on the substrate.

The substrate may be rotatable about an axis. The axis of rotation may or may not be an axis through the center of the substrate. In some instances, the systems, devices, and apparatus described herein may further comprise an automated or manual rotational unit configured to rotate the substrate. The rotational unit may comprise a motor and/or a rotor to rotate the substrate. For instance, the substrate may be affixed to a chuck (such as a vacuum chuck). The substrate may be rotated at a rotational speed of at least 1 revolution per minute (rpm), at least 2 rpm, at least 5 rpm, at least 10 rpm, at least 20 rpm, at least 50 rpm, at least 100 rpm, at least 200 rpm, at least 500 rpm, at least 1,000 rpm, at least 2,000 rpm, at least 5,000 rpm, at least 10,000 rpm, or greater. Alternatively or in addition, the substrate may be rotated at a rotational speed of at most about 10,000 rpm, 5,000 rpm, 2,000 rpm, 1,000 rpm, 500 rpm, 200 rpm, 100 rpm, 50 rpm, 20 rpm, 10 rpm, 5 rpm, 2 rpm, 1 rpm, or less. The substrate may be configured to rotate with a rotational velocity that is within a range defined by any two of the preceding values. The substrate may be configured to rotate with different rotational velocities during different operations described herein. The substrate may be configured to rotate with a rotational velocity that varies according to a time-dependent function, such as a ramp, sinusoid, pulse, or other function or combination of functions. The time-varying function may be periodic or aperiodic.

Analytes or reagents may be immobilized to the substrate during rotation. Analytes or reagents may be dispensed onto the substrate prior to or during rotation of the substrate. When the substrate is rotated at a relatively high rotational velocity, high speed coating across the substrate may be achieved via tangential inertia directing unconstrained spinning reagents in a partially radial direction (that is, away from the axis of rotation) during rotation, a phenomenon commonly referred to as centrifugal force. In some cases, the substrate may be rotated at relatively low velocities such that reagents dispensed to a certain location do not move to another location, or moves minimally, because of the rotation, to permit controlled dispensing of reagents to desired locations. For controlled dispensing, the substrate may be rotating with a rotational frequency of no more than 60 rpm, no more than 50 rpm, no more than 40 rpm, no more than 30 rpm, no more than 25 rpm, no more than 20 rpm, no more than 15 rpm, no more than 14 rpm, no more than 13 rpm, no more than 12 rpm, no more than 11 rpm, no more than 10 rpm, no more than 9 rpm, no more than 8 rpm, no more than 7 rpm, no more than 6 rpm, no more than 5 rpm, no more than 4 rpm, no more than 3 rpm, no more than 2 rpm, or no more than 1 rpm. In some cases the rotational frequency may be within a range defined by any two of the preceding values. In some cases the substrate may be rotating with a rotational frequency of about 5 rpm during controlled dispensing. A speed of substrate rotation may be adjusted according to the appropriate operation (e.g., high speed for spin-coating, high speed for washing the substrate, low speed for sample loading, low speed for detection, etc.).

In some cases, the substrate may be movable in any vector or direction. For example, such motion may be non-linear (e.g., in rotation about an axis), linear, or a hybrid of linear and non-linear motion. In some instances, the systems, devices, and apparatus described herein may further comprise a motion unit configured to move the substrate. The motion unit may comprise any mechanical component, such as a motor, rotor, actuator, linear stage, drum, roller, pulleys, etc., to move the substrate. Analytes or reagents may be immobilized to the substrate during any such motion. Analytes or reagents may be dispensed onto the substrate prior to, during, or subsequent to motion of the substrate.

### Loading Reagents onto an Open Substrate

The surface of the substrate may be in fluid communication with at least one fluid nozzle (of a fluid channel). The surface may be in fluid communication with the fluid nozzle via a non-solid gap, e.g., an air gap. In some cases, the surface may additionally be in fluid communication with at least one fluid outlet. The surface may be in fluid communication with the fluid outlet via an air gap. The nozzle may be configured to direct a solution to the array. The outlet may be configured to receive a solution from the substrate surface. The solution may be directed to the surface using one or more dispensing nozzles. For example, the solution may be directed to the array using at least 1, 2, 3, 4, 5, 6, 7, 8, 9, 10, 11, 12, 13, 14, 15, 16, 17, 18, 19, 20 or more dispensing nozzles. The solution may be directed to the array using a number of nozzles that is within a range defined by any two of the preceding values. In some cases, different reagents (e.g., nucleotide solutions of different types, different probes, washing solutions, etc.) may be dispensed via different nozzles, such as to prevent contamination. Each nozzle may be connected to a dedicated fluidic line or fluidic valve, which may further prevent contamination. A type of reagent may be dispensed via one or more nozzles. The one or more nozzles may be directed at or in proximity to a center of the substrate. Alternatively, the one or more nozzles may be directed at or in proximity to a location on the substrate other than the center of the substrate. Alternatively or in combination, one or more nozzles may be directed closer to the center of the substrate than one or more of the other nozzles. For instance, one or more nozzles used for dispensing washing reagents may be directed closer to the center of the substrate than one or more nozzles used for dispensing active reagents. The one or more nozzles may be arranged at different radii from the center of the substrate. Two or more nozzles may be operated in combination to deliver fluids to the substrate more efficiently. One or more nozzles may be configured to deliver fluids to the substrate as a jet, spray (or other dispersed fluid), and/or droplets. One or more nozzles may be operated to nebulize fluids prior to delivery to the substrate. For example, the fluids may be delivered as aerosol particles.

In some cases, the solution may be dispensed on the substrate while the substrate is stationary; the substrate may then be subjected to rotation (or other motion) following the dispensing of the solution. Alternatively, the substrate may be subjected to rotation (or other motion) prior to the dispensing of the solution; the solution may then be dispensed on the substrate while the substrate is rotating (or otherwise moving). In some cases, rotation of the substrate may yield a centrifugal force (or inertial force directed away from the axis) on the solution, causing the solution to flow radially outward over the array. In this manner, rotation of the substrate may direct the solution across the array. Continued rotation of the substrate over a period of time may dispense a fluid film (fluid layer) of a nearly constant thickness across the array.

One or more conditions such as the rotational velocity of the substrate, the acceleration of the substrate (e.g., the rate of change of velocity), viscosity of the solution, angle of dispensing (e.g., contact angle of a stream of reagents) of the solution, radial coordinates of dispensing of the solution (e.g., on center, off center, etc.), temperature of the substrate, temperature of the solution, and other factors may be adjusted and/or otherwise optimized to attain a desired wetting on the substrate and/or a film thickness on the substrate, such as to facilitate uniform coating of the substrate. For instance, one or more conditions may be applied to attain a film thickness of at least 10 nanometers (nm), 20 nm, 50 nm, 100 nm, 200 nm, 500 nm, 1 micrometer (μm), 2 μm, 5 μm, 10 μm, 15 μm, 20 μm, 50 μm, 100 μm, 200 μm, 500 μm, 1 millimeter (mm), or more. Alternatively or in addition, one or more conditions may be applied to attain a film thickness of at most 10 nanometers (nm), 20 nm, 50 nm, 100 nm, 200 nm, 500 nm, 1 micrometer (μm), 2 μm, 5 μm, 10 μm, 15 μm, 20 μm, 50 μm, 100 μm, 200 μm, 500 μm, 1 millimeter (mm) or less. One or more conditions may be applied to attain a film thickness that is within a range defined by any two of the preceding values. The thickness of the film may be measured or monitored by a variety of techniques, such as thin film spectroscopy with a thin film spectrometer, such as a fiber spectrometer. In some cases, a surfactant may be added to the solution, or a surfactant may be added to the surface to facilitate uniform coating or to facilitate sample loading efficiency. Alternatively or in conjunction, the thickness of the solution may be adjusted using mechanical, electric, physical, or other mechanisms. For example, the solution may be dispensed onto a substrate and subsequently leveled using, e.g., a physical scraper such as a squeegee, to obtain a desired thickness of uniformity across the substrate.

Reagents may be dispensed to the substrate to multiple locations, and/or multiple reagents may be dispensed to the substrate to a single location, via different mechanisms. Reagent dispensing mechanisms disclosed herein may be applicable to sample dispensing. For example, a reagent may comprise the sample. The term “loading onto a substrate,” as used in reference to a reagent or a sample herein, may refer to dispensing of the reagent or the sample to a surface of the substrate in accordance with any reagent dispensing mechanism described herein.

In some cases, dispensing may be achieved via relative motion of the substrate and the dispenser (e.g., nozzle). For example, a reagent may be dispensed to the substrate at a first location, and thereafter travel to a second location different from the first location due to forces (e.g., centrifugal forces, centripetal forces, inertial forces, etc.) caused by motion of the substrate (e.g., rotational motion of the substrate, linear motion of the substrate, combination thereof, etc.). In another example, a reagent may be dispensed to a reference location, and the substrate may be moved relative to the reference location such that the reagent is dispensed to multiple locations of the substrate. In another example, a dispenser may be moved relative to the substrate to dispense the reagent at different locations, for example moved prior to, during, or subsequent to dispensing. In an example, a reagent is ‘painted’ onto the substrate by moving the dispenser and/or the substrate relative to each other, along a desired path on the substrate. The open substrate geometry may allow for flexible and controlled dispensing of a reagent to a desired location on the substrate. In some cases, dispensing may be achieved without relative motion between the substrate and the dispenser. For example, multiple dispensers may be used to dispense reagents to different locations, and/or multiple reagents to a single location, or a combination thereof (e.g., multiple reagents to multiple locations).

In another example, an external force (e.g., involving a pressure differential, involving physical force, involving a magnetic force, involving an electrical force, etc.), such as wind, a field-generating device, or a physical device, may be applied to one or more surfaces of the substrate to direct reagents to different locations across the substrate. In another example, the method for dispensing reagents may comprise vibration. In such an example, reagents may be distributed or dispensed onto a single region or multiple regions of the substrate (or a surface of the substrate). The substrate (or a surface thereof) may then be subjected to vibration, which may spread the reagent to different locations across the substrate (or the surface). Alternatively or in conjunction, the method may comprise using mechanical, electric, physical, or other mechanisms to dispense reagents to the substrate. For example, the solution may be dispensed onto a substrate and a physical scraper (e.g., a squeegee) may be used to spread the dispensed material or spread the reagents to different locations and/or to obtain a desired thickness or uniformity across the substrate. Beneficially, such flexible dispensing may be achieved without contamination of the reagents.

In some instances, where a volume of reagent is dispensed to the substrate at a first location, and thereafter travels to a second location different from the first location, the volume of reagent may travel in a path or paths, such that the travel path or paths are coated with the reagent. In some cases, such travel path or paths may encompass a desired surface area (e.g., entire surface area, partial surface area(s), etc.) of the substrate. In some instances, two or more reagents may be mixed on the surface of the substrate, such as by being dispensed at the same location and/or by directing a first reagent to travel to meet additional reagent(s). In some instances, the mixture of reagents formed on the substrate may be homogenous or substantially homogenous. The mixture of reagents may be formed at a first location on the substrate prior to dispersing the mixing of reagents to other locations on the substrate, such as at locations to meet other reagents or analytes.

In some embodiments, one or more solutions may be delivered directly to the reaction site without substantial displacement of the one or more solution from the point of delivery. Methods of direct delivery of a solution to the reaction site may include aerosol delivery of the solution, applying the solution using an applicator, curtain-coating the solution, slot-die coating, dispensing the solution from a translating dispense probe, dispensing the solution from an array of dispense probes, dipping the substrate into the solution, or contacting the substrate to a sheet comprising the solution.

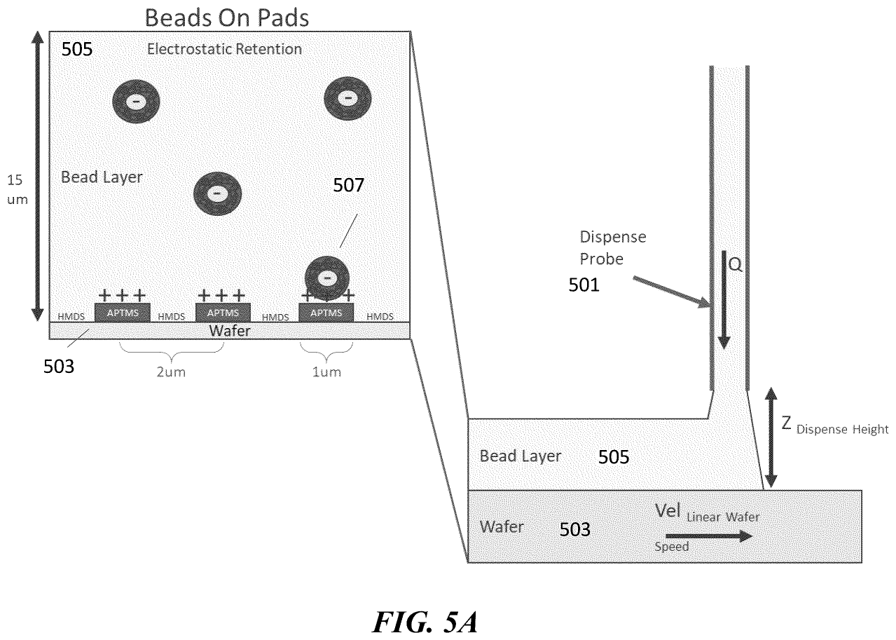

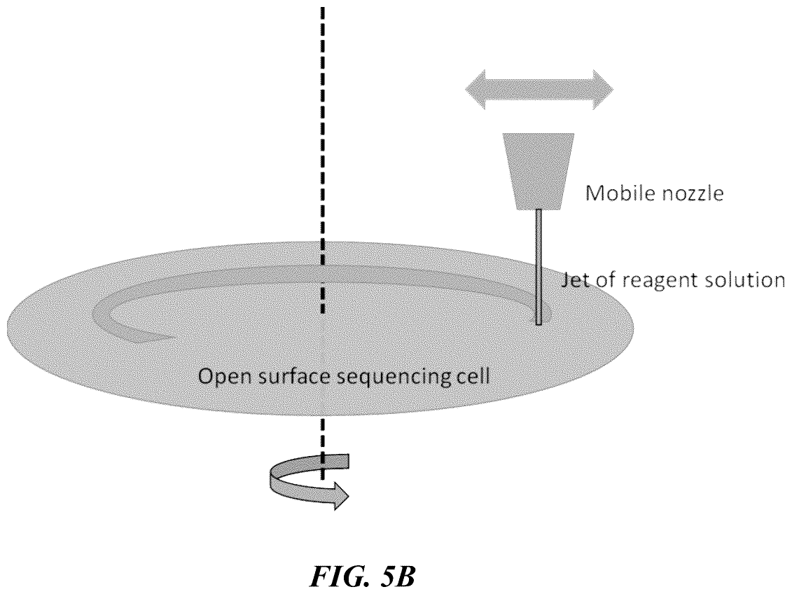

Aerosol delivery may comprise delivering a solution to the substrate in aerosol form by directing the solution to the substrate using a pressure nozzle or an ultrasonic nozzle. Applying the solution using an applicator may comprise contacting the substrate with an applicator comprising the solution and translating the applicator relative to the substrate. For example, applying the solution using an applicator may comprise painting the substrate. The solution may be applied in a pattern by translating the applicator, rotating the substrate, translating the substrate, or a combination thereof. Curtain-coating may comprise dispensing the solution from a dispense probe to the substrate in a continuous stream (e.g., a curtain or a flat sheet) and translating the dispense probe relative to the substrate. A solution may be curtain-coated in a pattern by translating the dispense probe, rotating the substrate, translating the substrate, or a combination thereof. Slot-die coating may comprise dispensing the solution from a dispense probe positioned near the substrate such that the solution forms a meniscus between the substrate and the dispense probe and translating the dispense probe relative to the substrate. A solution may be slot-die coated in a pattern by translating the dispense probe, rotating the substrate, translating the substrate, or a combination thereof. Dispensing the solution from a translating dispense probe may comprise translating the dispense probe relative to the substrate in a pattern (e.g., a spiral pattern, a circular pattern, a linear pattern, a striped pattern, a cross-hatched pattern, or a diagonal pattern). Dispensing the solution from an array of dispense probes may comprise dispensing the solution from an array of nozzles (e.g., a shower head) positioned above the substrate such that the solution is dispensed across an area of the substrate substantially simultaneously. Dipping the substrate into the solution may comprise dipping the substrate into a reservoir comprising the solution. In some embodiments, the reservoir may be a shallow reservoir to reduce the volume of the solution required to coat the substrate. Contacting the substrate to a sheet comprising the solution may comprise bringing the substrate in contact with a sheet of material (e.g., a porous sheet or a fibrous sheet) permeated with the solution. The solution may be transferred to the substrate. In some embodiments, the sheet of material may be a single-use sheet. In some embodiments, the sheet of material may be a reusable sheet. In some embodiments, a solution may be dispensed onto a substrate using the method illustrated in FIG. 5B, where a jet of a solution may be dispensed from a nozzle to a rotating substrate. The nozzle may translate radially relative to the rotating substrate, thereby dispensing the solution in a spiral pattern onto the substrate.

One or more solutions or reagents may be delivered to a substrate by any of the delivery methods disclosed herein. In some embodiments, two or more solutions or reagents are delivered to the substrate using the same or different delivery methods. In some embodiments, two or more solutions are delivered to the substrate such that the time between contacting a solution or reagent and a subsequent solution or reagent is substantially similar for each region of the substrate contacted to the one or more solutions or reagents. In some embodiments, a solution or reagent may be delivered as a single mixture. In some embodiments, the solution or reagent may be dispensed in two or more component solutions. For example, each component of the two or more component solutions may be dispensed from a distinct nozzle. The distinct nozzles may dispense the two or more component solutions substantially simultaneously to substantially the same region of the substrate such that a homogenous solution forms on the substrate. In some embodiments, dispensing of each component of the two or more components may be temporally separated. Dispensing of each component may be performed using the same or different delivery methods. In some embodiments, direct delivery of a solution or reagent may be combined with spin-coating.

A solution may be incubated on the substrate for any desired duration (e.g., minutes, hours, etc.). In some embodiments, the solution may be incubated on the substrate under conditions that maintain a layer of fluid on the surface. One or more of the temperature of the chamber, the humidity of the chamber, the rotation of the substrate, or the composition of the fluid may be adjusted such that the layer of fluid is maintained during incubation. In some instances, during incubation, the substrate may be rotated at an rotational frequency of no more than 60 rpm, 50 rpm, 40 rpm, 30 rpm, 25 rpm, 20 rpm, 15 rpm, 14 rpm, 13 rpm, 12 rpm, 11 rpm, 10 rpm, 9 rpm, 8 rpm, 7 rpm, 6 rpm, 5 rpm, 4 rpm, 3 rpm, 2 rpm, 1 rpm or less. In some cases, the substrate may be rotating with a rotational frequency of about 5 rpm during incubation.

The substrate or a surface thereof may comprise other features that aid in solution or reagent retention on the substrate or thickness uniformity of the solution or reagent on the substrate. In some cases, the surface may comprise a raised edge (e.g., a rim) which may be used to retain solution on the surface. The surface may comprise a rim near the outer edge of the surface, thereby reducing the amount of the solution that flows over the outer edge.

The dispensed solution may comprise any sample or any analyte disclosed herein. The dispensed solution may comprise any reagent disclosed herein. In some cases, the solution may be a reaction mixture comprising a variety of components. In some cases, the solution may be a component of a final mixture (e.g., to be mixed after dispensing). In non-limiting examples, the solution can comprise samples, analytes, supports, beads, probes, nucleotides, oligonucleotides, labels (e.g., dyes), terminators (e.g., blocking groups), other components to aid, accelerate, or decelerate a reaction (e.g., enzymes, catalysts, buffers, saline solutions, chelating agents, reducing agents, other agents, etc.), washing solution, cleavage agents, combinations thereof, deionized water, and other reagents and buffers.

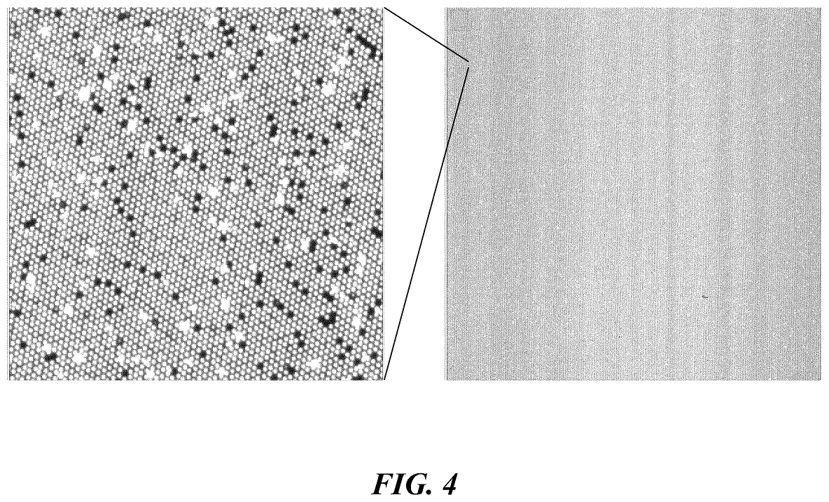

In some cases, a sample may be diluted such that the approximate occupancy of the individually addressable locations is controlled. In some cases, a sample may comprise beads, as described elsewhere herein, for example beads comprising nucleic acid colonies bound thereto. In some cases, an order of magnitude of at least about 10, 100, 1000, 10,000, 100,000, 1,000,000, 10,000,000, 100,000,000, 1,000,000,000, 10,000,000,000, 100,000,000,000 or more beads may be loaded on the substrate, such as to immobilize to as many individually addressable locations. Alternatively or in addition, an order of magnitude of at most about 100,000,000,000, 10,000,000,000, 1,000,000,000, 100,000,000, 10,000,000, 1,000,000, 100,000, 10,000, 1000, 100, or 10 beads may be loaded on the substrate, such as to immobilize to as many individually addressable locations. In some cases, the beads may be distinguishable from one another using a property of the beads, such as color, reflectance, anisotropy, brightness, fluorescence, etc. In some cases, as described elsewhere herein, different beads may comprise different tags (e.g., nucleic acid sequences) coupled thereto. For example, a bead may comprise an oligonucleotide molecule comprising a tag that identifies a bead amongst a plurality of beads. FIG. 4 illustrates images of a portion of a substrate surface after loading a sample containing beads onto a substrate patterned with a substantially hexagonal lattice of individually addressable locations, where the right panel illustrates a zoomed-out image of a portion of a surface, and the left panel illustrates a zoomed-in image of a section of the portion of the surface. In some cases, after sample loading, a “bead occupancy” may generally refer to the number of individually addressable locations of a type comprising at least one bead out of the total number of individually addressable locations of the same type. A bead “landing efficiency” may generally refer to the number of beads that bind to the surface out of the total number of beads dispensed on the surface.

In some cases, beads may be dispensed to the substrate according to one or more systems and methods shown in FIGS. 5A-5B. As shown in FIG. 5A, a solution comprising beads may be dispensed from a dispense probe 501 (e.g., a nozzle) to a substrate 503 (e.g., a wafer) to form a layer 505. The dispense probe may be positioned at a height (“Z”) above the substrate. In the illustrated example, the beads are retained in the layer 505 by electrostatic retention, and may immobilize to the substrate at respective individually addressable locations. A set of beads in the solution may each comprise a population of amplified products (e.g., nucleic acid molecules) immobilized thereto, which amplified products accumulate to a negative charge on the bead with affinity to a positive charge. Otherwise, the beads may comprise reagents that have a negative charge. The substrate comprises alternating surface chemistry between distinguishable locations, in which a first location type comprises APTMS carrying a positive charge with affinity towards the negative charge of the amplified bead (e.g., a bead comprising amplified products immobilized thereto, and as distinguished from a negative bead which does not the comprise the same) or other bead comprising the negative charge, and a second location type comprises HMDS which has lower affinity and/or is repellant of the amplified bead or other bead comprising the negative charge. Within the layer 505 a bead may successfully land on a first location of the first location type (as in 507). In the illustrated example, the location size is 1 micron, the pitch between the different locations of the same location type (e.g., first location type) is 2 microns, and the layer has a depth of 15 micron. FIG. 5B illustrates a reagent (e.g., beads) being dispensed along a path on an open surface of the substrate. As shown in FIG. 5B, a reagent solution may be dispensed from a dispense probe (e.g., a nozzle). The reagent may be dispensed on the surface in any desired pattern or path. This may be achieved by moving one or both of the substrate and the dispense nozzle. The substrate and the dispense probe may move in any configuration with respect to each other to achieve any pattern (e.g., linear pattern, substantially spiral pattern, etc.).

In some instances, a subset or an entirety of the solution(s) may be recycled after the solution(s) have contacted the substrate. Recycling may comprise collecting, filtering, and reusing the subset or entirety of the solution. The filtering may be molecule filtering.

### Detection

An optical system comprising a detector may be configured to detect one or more signals from a detection area on the substrate prior to, during, or subsequent to, the dispensing of reagents to generate an output. Signals from multiple individually addressable locations may be detected during a single detection event. Signals from the same individually addressable location may be detected in multiple instances.

A detectable signal, such as an optical signal (e.g., fluorescent signal), may be generated upon a reaction between a probe in the solution and the analyte. For example, the signal may originate from the probe and/or the analyte. The detectable signal may be indicative of a reaction or interaction between the probe and the analyte. The detectable signal may be a non-optical signal. For example, the detectable signal may be an electronic signal. The detectable signal may be detected by a detector (e.g., one or more sensors). For example, an optical signal may be detected via one or more optical detectors in an optical detection scheme described elsewhere herein. The signal may be detected during rotation of the substrate. The signal may be detected following termination of the rotation. The signal may be detected while the analyte is in fluid contact with a solution. The signal may be detected following washing of the solution. In some instances, after the detection, the signal may be muted, such as by cleaving a label from the probe and/or the analyte, and/or modifying the probe and/or the analyte. Such cleaving and/or modification may be affected by one or more stimuli, such as exposure to a chemical, an enzyme, light (e.g., ultraviolet light), or temperature change (e.g., heat). In some instances, the signal may otherwise become undetectable by deactivating or changing the mode (e.g., detection wavelength) of the one or more sensors, or terminating or reversing an excitation of the signal. In some instances, detection of a signal may comprise capturing an image or generating a digital output (e.g., between different images).

The operations of (i) directing a solution to the substrate and (ii) detection of one or more signals indicative of a reaction between a probe in the solution and an analyte immobilized to the substrate, may be repeated any number of times. Such operations may be repeated in an iterative manner. For example, the same analyte immobilized to a given location in the array may interact with multiple solutions in the multiple repetition cycles. For each iteration, the additional signals detected may provide incremental, or final, data about the analyte during the processing. For example, where the analyte is a nucleic acid molecule and the processing is sequencing, additional signals detected for each iteration may be indicative of a base in the nucleic acid sequence of the nucleic acid molecule. In some cases, multiple solutions can be provided to the substrate without intervening detection events. In some cases, multiple detection events can be performed after a single flow of solution. In some instances, a washing solution, cleaving solution (e.g., comprising cleavage agent), and/or other solutions may be directed to the substrate between each operation, between each cycle, or a certain number of times for each cycle.

The optical system may be configured for continuous area scanning of a substrate during rotational motion of the substrate. The term “continuous area scanning (CAS),” as used herein, generally refers to a method in which an object in relative motion is imaged by repeatedly, electronically or computationally, advancing (clocking or triggering) an array sensor at a velocity that compensates for object motion in the detection plane (focal plane). CAS can produce images having a scan dimension larger than the field of the optical system. TDI scanning may be an example of CAS in which the clocking entails shifting photoelectric charge on an area sensor during signal integration. For a TDI sensor, at each clocking step, charge may be shifted by one row, with the last row being read out and digitized. Other modalities may accomplish similar function by high speed area imaging and co-addition of digital data to synthesize a continuous or stepwise continuous scan.

The optical system may comprise one or more sensors. The sensors may detect an image optically projected from the sample. The optical system may comprise one or more optical elements. An optical element may be, for example, a lens, prism, mirror, wave plate, filter, attenuator, grating, diaphragm, beam splitter, diffuser, polarizer, depolarizer, retroreflector, spatial light modulator, or any other optical element. The system may comprise any number of sensors. In some cases, a sensor is any detector as described herein. In some examples, the sensor may comprise image sensors, CCD cameras, CMOS cameras, TDI cameras (e.g., TDI line-scan cameras), pseudo-TDI rapid frame rate sensors, or CMOS TDI or hybrid cameras. The optical system may further comprise any optical source. In some cases, where there are multiple sensors, the different sensors may image the same or different regions of the rotating substrate, in some cases simultaneously. Each sensor of the plurality of sensors may be clocked at a rate appropriate for the region of the rotating substrate imaged by the sensor, which may be based on the distance of the region from the center of the rotating substrate or the tangential velocity of the region. In some cases, multiple scan heads can be operated in parallel along different imaging paths (e.g., interleaved spiral scans, nested spiral scans, interleaved ring scans, nested ring scans). A scan head may comprise one or more of a detector element such as a camera (e.g., a TDI line-scan camera), an illumination source (e.g., as described herein), and one or more optical elements (e.g., as described herein).

The system may further comprise a controller. The controller may be operatively coupled to the one or more sensors. The controller may be programmed to process optical signals from each region of the rotating substrate. For instance, the controller may be programmed to process optical signals from each region with independent clocking during the rotational motion. The independent clocking may be based at least in part on a distance of each region from a projection of the axis and/or a tangential velocity of the rotational motion. The independent clocking may be based at least in part on the angular velocity of the rotational motion. While a single controller has been described, a plurality of controllers may be configured to, individually or collectively, perform the operations described herein.

In some cases, the optical system may comprise an immersion objective lens. The immersion objective lens may be in contact with an immersion fluid that is in contact with the open substrate. The immersion fluid may comprise any suitable immersion medium for imaging (e.g., water, aqueous, organic solution). In some cases, an enclosure may partially or completely surround a sample-facing end of the optical imaging objective. The enclosure may be configured to contain the fluid. The enclosure may not be in contact with the substrate; for example, a gap between the enclosure and the substrate may be filled by the fluid contained by the enclosure (e.g., the enclosure can retain the fluid via surface tension). In some cases, an electric field may be used to regulate a hydrophobicity of one or more surfaces of the container to retain at least a portion of the fluid contacting the immersion objective lens and the open substrate

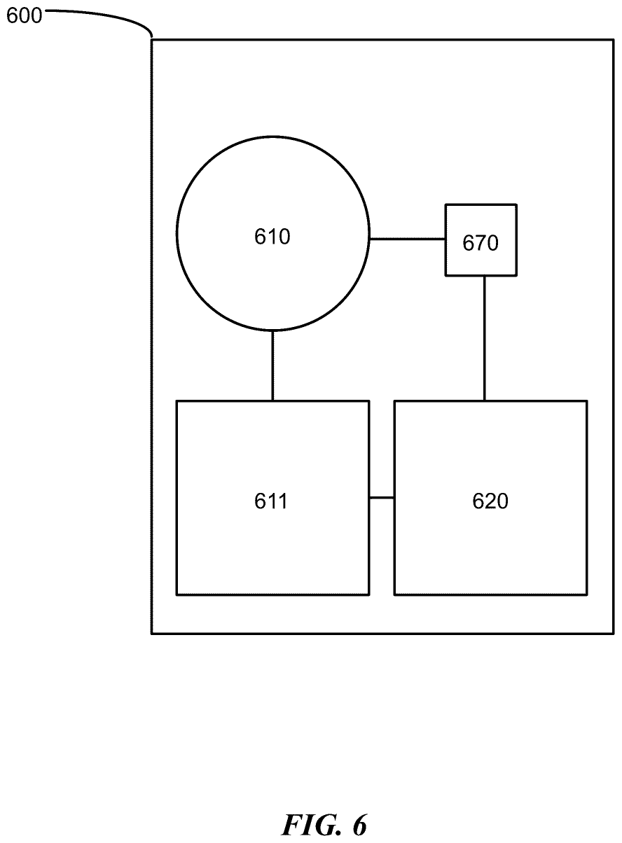

FIG. 6 shows a computerized system 600 for sequencing a nucleic acid molecule. The system may comprise a substrate 610, such as any substrate described herein. The system may further comprise a fluid flow unit 611. The fluid flow unit may comprise any element associated with fluid flow described herein. The fluid flow unit may be configured to direct a solution comprising a plurality of nucleotides described herein to an array of the substrate prior to or during rotation of the substrate. The fluid flow unit may be configured to direct a washing solution described herein to an array of the substrate prior to or during rotation of the substrate. In some instances, the fluid flow unit may comprise pumps, compressors, and/or actuators to direct fluid flow from a first location to a second location. The fluid flow unit may be configured to direct any solution to the substrate 610. The fluid flow system may be configured to collect any solution from the substrate 610. The system may further comprise a detector 670, such as any detector described herein. The detector may be in sensing communication with the substrate surface.

The system may further comprise one or more processors 620. The one or more processors may be individually or collectively programmed to implement any of the methods described herein. For instance, the one or more processors may be individually or collectively programmed to implement any or all operations of the methods of the present disclosure. In particular, the one or more processors may be individually or collectively programmed to: (i) direct the fluid flow unit to direct the solution comprising the plurality of nucleotides across the array during or prior to rotation of the substrate; (ii) subject the nucleic acid molecule to a primer extension reaction under conditions sufficient to incorporate at least one nucleotide from the plurality of nucleotides into a growing strand that is complementary to the nucleic acid molecule; and (iii) use the detector to detect a signal indicative of incorporation of the at least one nucleotide, thereby sequencing the nucleic acid molecule.

### High Throughput

An open substrate system of the present disclosure may comprise a barrier system configured to maintain a fluid barrier between a sample processing environment and an exterior environment. The barrier system is described in further detail in U.S. Patent Pub. No. 2021/0354126, which is entirely incorporated herein by reference. A sample environment system may comprise a sample processing environment defined by a chamber and a lid plate, where the lid plate is not in contact with the chamber. The gap between the lid plate and the chamber may comprise the fluid barrier. The fluid barrier may comprise fluid (e.g., air) from the sample processing environment and/or the exterior environment and may have lower pressure than the sample environment, the external environment, or both. The fluid in the fluid barrier may be in coherent motion or bulk motion.

The sample processing environment may comprise therein a substrate, such as any substrate described elsewhere herein. Any operation performed on or with the substrate, as described elsewhere herein, may be performed within the sample processing environment while the fluid barrier is maintained. For example, the substrate may be rotated within the sample processing environment during various operations. In another example, fluid may be directed to the substrate while the substrate is in the sample processing environment, via a fluid handler (e.g., nozzle) that penetrates the lid plate into the sample processing environment. In another example, a detector can image the substrate while the substrate is in the sample processing environment, via a detector that penetrates the lid plate into the sample processing environment. Beneficially, the fluid barrier may help maintain temperature(s) and/or relative humidit(ies), or ranges thereof, within the sample processing environment during various processing operations.

The systems described herein, or any element thereof, may be environmentally controlled. For instance, the systems may be maintained at a specified temperature or humidity. For an operation, the systems (or any element thereof) may be maintained at a temperature of at least 20 degrees Celsius (° C.), 25° C., 30° C., 35° C., 40° C., 45° C., 50° C., 55° C., 60° C., 65° C., 70° C., 75° C., 80° C., 85° C., 90° C., 95° C., 100° C., or more. Alternatively or in addition, for an operation, the systems (or any element thereof) may be maintained at a temperature of at most 100° C., 95° C., 90° C., 85° C., 80° C., 75° C., 70° C., 65° C., 60° C., 55° C., 50° C., 45° C., 40° C., 35° C., 30° C., 25° C., 20° C., or less. Different elements of the system may be maintained at different temperatures or within different temperature ranges, such as the temperatures or temperature ranges described herein. Elements of the system may be set at temperatures above the dew point to prevent condensation. Elements of the system may be set at temperatures below the dew point to collect condensation. In one example, a sample processing environment comprising a substrate as described elsewhere herein may be environmentally controlled from an exterior environment. The sample processing environment may be further divided into separate regions which are maintained at different local temperatures and/or relative humidities, such as a first region contacting or in proximity to a surface of the substrate, and a second region contacting or in proximity to a top portion of the sample processing environment (e.g., a lid). For example, the local environment of the first region may be maintained at a first set of temperatures and first set of humidities configured to prevent or minimize evaporation of one or more reagents on the surface of the substrate, and the local environment of the second region may be maintained at a second set of temperatures and second set of humidities configured to enhance or restrict condensation. The first set of temperatures may be the lowest temperatures within the sample processing environment and the second set temperatures may be the highest temperatures within the sample processing environment.

In some instances, the environmental conditions of the different regions may be achieved by controlling the temperature of the enclosure. In some instances, the environmental conditions of the different regions may be achieved by controlling the temperature of selected parts or whole of the container. In some instances, the environmental conditions of the different regions may be achieved by controlling the temperature of selected parts or whole of the substrate. In some instances, the environmental conditions of the different regions may be achieved by controlling the temperature of reagents dispensed to the substrate. Any combination thereof may be used to control the environmental conditions of the different regions. Heat transfer may be achieved by any method, including for example, conductive, convective, and radiative methods.

While examples described herein provide relative rotational motion of the substrates and/or detector systems, the substrates and/or detector systems may alternatively or additionally undergo relative non-rotational motion, such as relative linear motion, relative non-linear motion (e.g., curved, arcuate, angled, etc.), and any other types of relative motion.

In some instances, an open substrate is retained in the same or approximately the same physical location during processing of an analyte and subsequent detection of a signal associated with a processed analyte.

In some instances, different operations on or with the open substrate are performed in different stations. Different stations may be disposed in different physical locations. For example, a first station may be disposed above, below, adjacent to, or across from a second station. In some cases, the different stations can be housed within an integrated housing. Alternatively, the different stations can be housed separately. In some cases, different stations may be separated by a barrier, such as a retractable barrier (e.g., sliding door). One or more different stations of a system, or portions thereof, may be subjected to different physical conditions, such as different temperatures, pressures, or atmospheric compositions. In an example, a processing station may comprise a first atmosphere comprising a first set of conditions and a second atmosphere comprising a second set of conditions. The barrier systems may be used to maintain different physical conditions of one or more different stations of the system, or portions thereof, as described elsewhere herein.

The open substrate may transition between different stations by transporting a sample processing environment containing the open substrate (such as the one described with respect to the barrier system) between the different stations. One or more mechanical components or mechanisms, such as a robotic arm, elevator mechanism, actuators, rails, and the like, or other mechanisms may be used to transport the sample processing environment.

An environmental unit (e.g., humidifiers, heaters, heat exchangers, compressors, etc.) may be configured to regulate one or more operating conditions in each station. In some instances, each station may be regulated by independent environmental units. In some instances, a single environmental unit may regulate a plurality of stations. In some instances, a plurality of environmental units may, individually or collectively, regulate the different stations. An environmental unit may use active methods or passive methods to regulate the operating conditions. For example, the temperature may be controlled using heating or cooling elements. The humidity may be controlled using humidifiers or dehumidifiers. In some instances, a part of a particular station, such as within a sample processing environment, may be further controlled from other parts of the particular station. Different parts may have different local temperatures, pressures, and/or humidity.

In one example, the delivery and/or dispersal of reagents may be performed in a first station having a first operating condition, and the detection process may be performed in a second station having a second operating condition different from the first operating condition. The first station may be at a first physical location in which the open substrate is accessible to a fluid handling unit during the delivery and/or dispersal processes, and the second station may be at a second physical location in which the open substrate is accessible to the detector system.

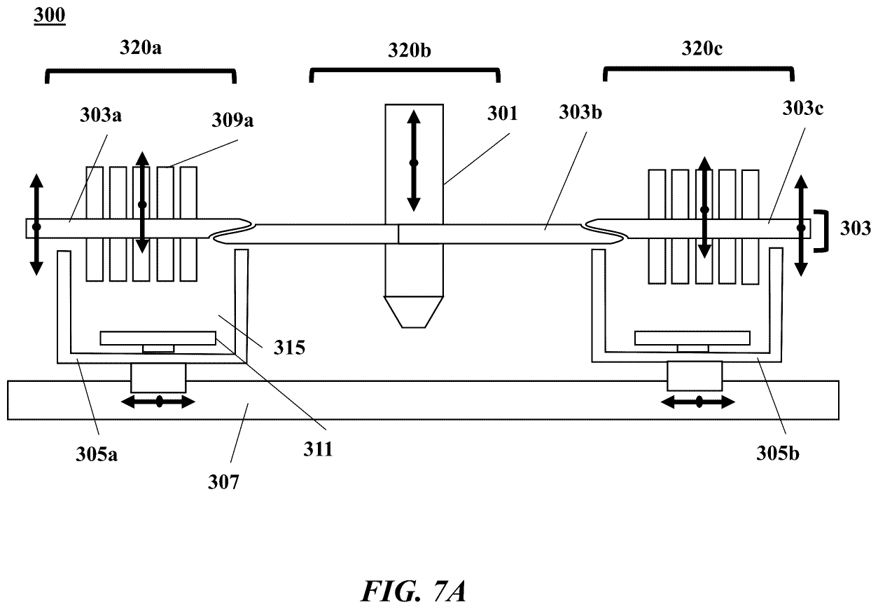

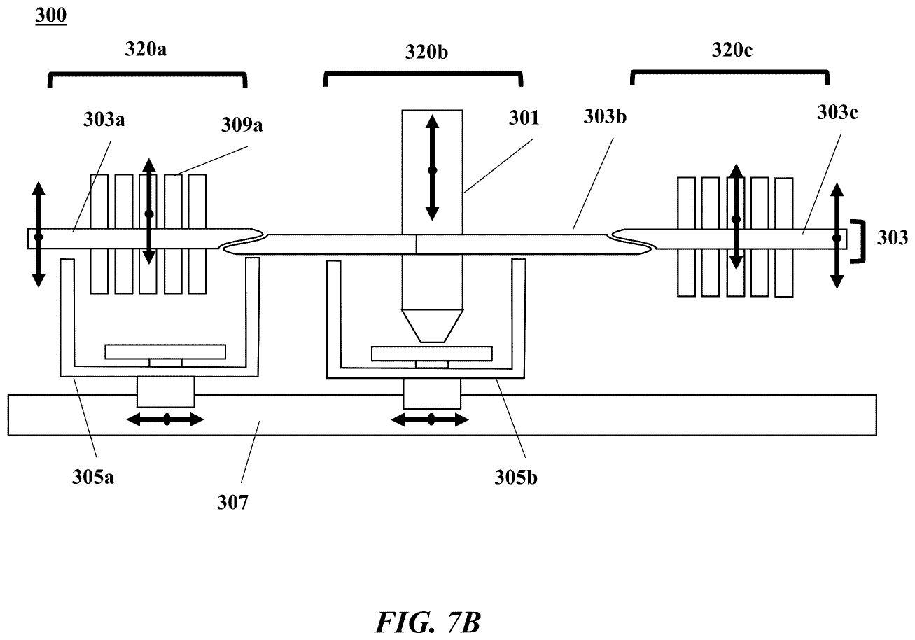

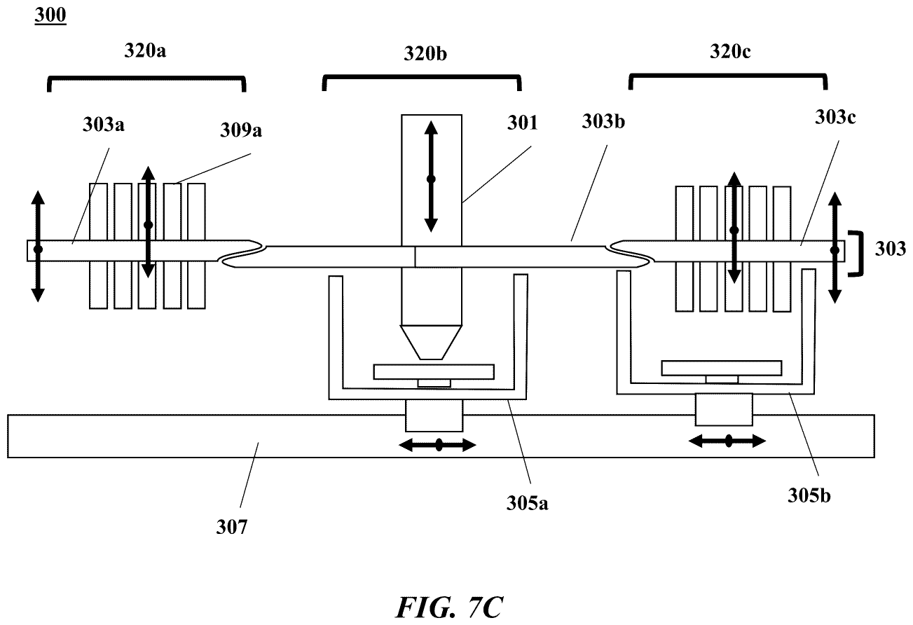

One or more modular sample environment systems (each having its own barrier system) can be used between the different stations. In some instances, the systems described herein may be scaled up to include two or more of a same station type. For example, a sequencing system may include multiple processing and/or detection stations. FIGS. 7A-7C illustrate a system 300 that multiplexes two modular sample environment systems in a three-station system. In FIG. 7B, a first chemistry station (e.g., 320a) can operate (e.g., dispense reagents, e.g., to incorporate nucleotides to perform sequencing by synthesis) via at least a first operating unit (e.g., fluid dispenser 309a) on a first substrate (e.g., 311) in a first sample environment system (e.g., 305a) while substantially simultaneously, a detection station (e.g., 320b) can operate (e.g., scan) on a second substrate in a second sample environment system (e.g., 305b) via at least a second operating unit (e.g., detector 301), while substantially simultaneously, a second chemistry station (e.g., 320c) sits idle. An idle station may not operate on a substrate. An idle station (e.g., 320c) may be recharged, reloaded, replaced, cleaned, washed (e.g., to flush reagents), calibrated, reset, kept active (e.g., power on), and/or otherwise maintained during an idle time. After an operating cycle is complete, the sample environment systems may be re-stationed, as in FIG. 7C, where the second substrate in the second sample environment system (e.g., 305b) is re-stationed from the detection station (e.g., 320b) to the second chemistry station (e.g., 320c) for operation (e.g., dispensing of reagents, e.g., to incorporate nucleotides to perform sequencing by synthesis) by the second chemistry station, and the first substrate in the first sample environment system (e.g., 305a) is re-stationed from the first chemistry station (e.g., 320a) to the detection station (e.g., 320b) for operation (e.g., scanning) by the detection station. An operating cycle may be deemed complete when operation at each active, parallel station is complete. During re-stationing, the different sample environment systems may be physically moved (e.g., along the same track or dedicated tracks, e.g., rail(s) 307) to the different stations and/or the different stations may be physically moved to the different sample environment systems. One or more components of a station, such as modular plates 303a, 303b, 303c of plate 303 defining a particular station(s), may be physically moved to allow a sample environment system to exit the station, enter the station, or cross through the station. During processing of a substrate at station, the environment of a sample environment region (e.g., 315) of a sample environment system (e.g., 305a) may be controlled and/or regulated according to the station's requirements. After the next operating cycle is complete, the sample environment systems can be re-stationed again, such as back to the configuration of FIG. 7B, and this re-stationing can be repeated (e.g., between the configurations of FIGS. 7B and 7C) with each completion of an operating cycle until the required processing for a substrate is completed. In this illustrative re-stationing scheme, the detection station may be kept active (e.g., not have idle time not operating on a substrate) for all operating cycles by providing alternating different sample environment systems to the detection station for each consecutive operating cycle. Beneficially, use of the detection station is optimized. Based on different processing or equipment needs, an operator may opt to run the two chemistry stations (e.g., 320a, 320c) substantially simultaneously while the detection station (e.g., 320b) is kept idle, such as illustrated in FIG. 7A.

Beneficially, different operations within the system may be multiplexed with high flexibility and control. For example, as described herein, one or more processing stations may be operated in parallel with one or more detection stations on different substrates in different modular sample environment systems to reduce or eliminate lag between different sequences of operations (e.g., chemistry first, then detection). The modular sample environment systems may be translated between the different stations accordingly to optimize efficient equipment use (e.g., such that the detection station is in operation almost 100% of the time). In some examples, at least 1, 2, 3, 4, 5, 6, 7, 8, 9, or more modules or stations of the sequencing system may be multiplexed. For example, 2 or more of the modules may each perform their intended function simultaneously or according to the methods described elsewhere herein. An example of this may comprise two-station multiplexing of an optics station and a chemistry station as described herein. Another example may comprise multiplexing three or more stations and process phases. For example, the method may comprise using staggered chemistry phases sharing a scanning station. The scanning station may be a high-speed scanning station. The modules or stations may be multiplexed using various sequences and configurations.

The nucleic acid sequencing systems and optical systems described herein (or any elements thereof) may be combined in a variety of architectures.

### Photolabile Spatial Labeling of Analytes

The present disclosure provides methods for generating spatial barcodes at individually addressable locations on a substrate. The spatial barcodes may be used to label an analyte. In some cases, spatial barcodes may be generated directly on analytes disposed at such individually addressable locations on a substrate. In some instances, a method may comprise (a) providing a substrate comprising a plurality of first polynucleotides; (b) providing a plurality of additional polynucleotides to the substrate; and (c) subjecting the substrate with illumination under conditions sufficient to link or couple at least a subset of the plurality of first polynucleotides with at least a subset of the plurality of additional polynucleotides. A plurality of ligation templates may be provided to assist linking of the different polynucleotides. Only one or more selective regions of the substrate may be subjected to illumination, and only the subset of the plurality of first polynucleotides located at the one or more selective regions may link or couple to the subset of the plurality of additional polynucleotides. The substrate may be washed to remove a plurality of additional polynucleotides unattached to any first polynucleotide, and optionally, (b) and (c) may be repeated any number of times to iteratively extend the plurality of first polynucleotides with additional plurality(ies) of additional polynucleotides to generate final spatial barcodes. An individual first polynucleotide may be extended with any number of additional polynucleotides to generate a final spatial barcode. By selectively illuminating different regions of the substrate, different first polynucleotides may be extended with different pluralities of additional polynucleotides, in any number of cycles, to generate a diverse pool of spatial barcodes on the substrate.

In some instances, the method may comprise: (a) providing a substrate, wherein the substrate comprises a plurality of individually addressable locations comprising a plurality of first polynucleotides immobilized thereto; (b) applying a fluid layer to said substrate, wherein the fluid layer comprises (i) a plurality of second polynucleotides and (ii) a plurality of ligation templates, under conditions sufficient to hybridize the plurality of first polynucleotides to the plurality of ligation templates and to hybridize said plurality of second polynucleotides to the plurality of ligation templates, wherein the fluid layer has a thickness of at most 50 micrometers; and (c) subsequent to hybridization in (b), selectively illuminating a subset of the plurality of individually addressable locations, under conditions sufficient to: (i) link a subset of said plurality of first polynucleotides at the subset of said plurality of individually addressable locations with a subset of the plurality of ligation templates, and (ii) link a subset of the plurality of second polynucleotides with the subset of the plurality of ligation templates.

The substrate may comprise a plurality of individually addressable locations, as described elsewhere herein. A plurality of first polynucleotides may be immobilized at the plurality of individually addressable locations. In some cases, the plurality of first polynucleotides are directly immobilized to a surface of the substrate. In other cases, the plurality of first polynucleotides are immobilized to (e.g., coupled to) one or more objects, which objects are immobilized at the plurality of individually addressable locations. For example, the plurality of first polynucleotides are coupled to a plurality of beads (e.g., one first polynucleotide on one bead), which plurality of beads are immobilized at the plurality of individually addressable locations (e.g., one bead on one individually addressable location). In some cases, the plurality of individually addressable locations may immobilize a plurality of analytes, and the plurality of analytes may be coupled to a plurality to first polynucleotides. In some instances, the method may further comprise prior to (a), providing to the substrate, which may or may not have a plurality of analytes immobilized thereto at the plurality of individually addressable locations, the plurality of first polynucleotides to immobilize the plurality of first polynucleotides at the plurality of individually addressable locations. The plurality of first polynucleotides may comprise identical sequences. The plurality of first polynucleotides may comprise different sequences. In some instances, the identities of the first polynucleotides in the plurality of first polynucleotides and/or their respective locations on the substrate may be known.

Spatial barcodes may be generated by attaching one or more additional polynucleotides to the first polynucleotides on the substrate. To generate a diverse pool of barcodes, different additional polynucleotide(s) may be attached to different first polynucleotides on the substrate. A plurality of n^{th} (e.g., second) polynucleotides may be provided to the substrate. A plurality of ligation templates may be provided to the substrate prior to, during, or subsequent to providing the plurality of n^{th} polynucleotides. The plurality of n^{th} polynucleotides may be provided to the entire substrate surface, to only a portion of the substrate surface including the one or more selective regions of the substrate that will be subjected to illumination, or to only the one or more selective regions of the substrate that will be subjected to illumination. The plurality of ligation templates may be provided to the entire substrate surface, to only a portion of the substrate surface including the one or more selective regions of the substrate that will be subjected to illumination, or to only the one or more selective regions of the substrate that will be subjected to illumination. Any reagents provided to the substrate according to the methods provided herein may be applied as a film or fluid layer. Systems and methods for dispensing reagents to an open substrate and achieving a film or fluid layer are described elsewhere herein. In some cases, the film may have a uniform thickness. In some cases, the film or fluid layer may have a maximum thickness of any range described elsewhere herein (e.g., at most 50 micrometers or at most 15 micrometers).

On the substrate, where available, the plurality of first polynucleotides and the plurality of second polynucleotides may hybridize to the plurality of ligation templates, such that plurality of first polynucleotides and plurality of second polynucleotides form a complex via the plurality of ligation templates. The hybridization may occur via complementary pairing (e.g., standard or non-standard DNA or RNA base pairing). The complex may be readily reversible to separate the first polynucleotide, second polynucleotide, and/or ligation template, by applying one or more stimuli (e.g., applying heat, adding a chemical solution, lowering a melting temperature, etc.).

After the complexes form, the substrate may be subjected to selective illumination. As used herein, the term “selective illumination” may generally refer to illumination of one or more selective regions as opposed to an entire region. The one or more selective regions may correspond to a subset of individually addressable locations of the plurality of individually addressable locations. The one or more selective regions may be predetermined and/or recorded as selected. The selective illumination may link a subset of the plurality of first polynucleotides at the subset of the plurality of individually addressable locations with a subset of the plurality of ligation templates hybridized thereto and/or link at least a subset of the plurality of second polynucleotides with the subset of the plurality of ligation templates hybridized thereto.

In some instances, the link may be a cross-link (also referred to herein as a cross link). In some cases, a cross-link may comprise a bond. The bond of the cross-link may comprise a covalent bond, a non-covalent bond, or combination thereof. In some cases, the cross-link may comprise a series of bonds, such as a series of covalent bonds and/or non-covalent bonds. In some cases, the cross-link may comprise at least 1, 2, 3, 4, 5, or more bonds. In some cases, the cross-link may comprise at most 1, 2, 3, 4, or 5 bonds. In some cases, a cross-link may comprise a chemical cross-link and/or a physical cross-link. In some cases, a cross-link may comprise coordination bonding, hydrogen bonding, ionic interaction, van der Waals interaction, or a combination thereof. In some cases, the cross-link may also comprise an oxidative cross-link. In some instances, the link may comprise a non-hydrogen bond. In some cases, the bond may be formed by an addition reaction (i.e., formation of a larger adduct from a plurality of molecules). In some cases, the addition reaction may comprise a polar addition reaction or a non-polar addition reaction. In some cases, the polar addition reaction may comprise an electrophilic addition, a nucleophilic addition, or a free-radical addition. In some cases, the non-polar addition reaction may comprise a cycloaddition.

In some instances, the bond may be formed by a cyclization reaction. In some cases, the bond may comprise a cycloaddition bond. In some cases, the cycloaddition bond may be concerted. In some cases, the cycloaddition bond may be pericyclic. In other cases, the cycloaddition bond may not be pericyclic. In some cases, the formation of the cycloaddition bond may comprise the formation of a carbon-carbon bond. In some cases, the formation of the cycloaddition may not comprise a nucleophile or an electrophile. In some cases, the formation of the cycloaddition may comprise the formation of a carbon-carbon bond absent the nucleophile or electrophile.

In some instances, a cycloaddition bond may be described by the formula [i+j+ . . . ], wherein the variables (e.g., i and j) represent the number of electrons involved in the formation of the adduct from two molecules. If an adduct is formed by more than two molecules, more variables may be denoted. In some cases, i may be 1, 2, 3, 4, 5, 6, 7, 8, 9, or more. In some cases, j may be 1, 2, 3, 4, 5, 6, 7, 8, 9, or more. In some instances, a cycloaddition bond may be described by the formula (i+j+ . . . ), wherein the variables (e.g., i and j) represent the number of linear atoms of each molecule forming the adduct. If an adduct is formed by more than two molecules, more variables may be denoted. In some cases, i may be 1, 2, 3, 4, 5, 6, 7, 8, 9, or more. In some cases, j may be 1, 2, 3, 4, 5, 6, 7, 8, 9, or more.

In some instances, the link may comprise photolabile formation of the cycloaddition bond. In some cases, the cycloaddition bond may comprise a photo-activated (or light-activated) cycloaddition bond. The photo-activated cycloaddition bond may be a photocycloaddition bond. In some cases, the photocycloaddition bond may comprise a [2+2] photoaddition bond. In some cases, the photocycloaddition bond may comprise a [1+1], [2+1], [3+1], [4+1], [5+1], [6+1], [1+2], [2+2], [3+2], [4+2], [5+2], [6+2], [1+3], [2+3], [3+3], [4+3], [5+3], [6+3], [1+4], [2+4], [3+4], [4+4], [5+4], [6+4], [1+5], [2+5], [3+5], [4+5], [5+5], [6+5], [1+6], [2+6], [3+6], [4+6], [5+6], or [6+6] photocycloaddition bond. In some cases, the photocycloaddition bond may comprise a (1+1), (2+1), (3+1), (4+1), (5+1), (6+1), (1+2), (2+2), (3+2), (4+2), (5+2), (6+2), (1+3), (2+3), (3+3), (4+3), (5+3), (6+3), (1+4), (2+4), (3+4), (4+4), (5+4), (6+4), (1+5), (2+5), (3+5), (4+5), (5+5), (6+5), (1+6), (2+6), (3+6), (4+6), (5+6), or (6+6) photocycloaddition bond. In some cases, the cycloaddition bond may comprise a thermal cycloaddition bond. In some cases, the cycloaddition bond may comprise a formal cycloaddition bond (e.g., a metal catalyzed or a non-metal catalyzed cycloaddition bond). In some cases, the formal cycloaddition bond may comprise a metal-catalyzed cycloaddition bond formation reaction. In some cases, the thermal or formal cycloaddition may be described by the formula [i+j+ . . . ] or (i+j+ . . . ) as described herein.

In some cases, the formation of the cycloaddition bond may comprise a 1,3-Dipolar cycloaddition, an alkyne trimerisation, an aza-Diels-Alder reaction, an azide-alkyne Huisgen cycloaddition, a Bradsher cycloaddition, a Cheletropic reaction, a Conia-ene reaction, a cyclopropanation, a diazoalkane 1,3-dipolar cycloaddition, a Diels-Alder reaction, an enone-alkene cycloadditions, a hexadehydro Diels-Alder reaction, an imine Diels-Alder reaction, an intramolecular Diels-Alder cycloaddition, an inverse electron-demand Diels-Alder reaction, a ketene cycloaddition, a McCormack reaction, a metal-centered cycloaddition reactions, a nitrone-olefin (3+2) cycloaddition, an oxo-Diels-Alder reaction, an ozonolysis, a Pauson-Khand reaction, a Povarov reaction, a Prato reaction, a retro-Diels-Alder reaction, a Staudinger synthesis, a trimethylenemethane cycloaddition, a vinylcyclopropane (5+2) cycloaddition, a Wagner-Jauregg reaction, Woodward-Hoffmann rules, or a combination thereof.

In some instances, the (i) link between the subset of the plurality of first polynucleotides at the subset of the plurality of individually addressable locations and a subset of the plurality of ligation templates hybridized thereto and the (ii) link between the subset of the plurality of second polynucleotides and a subset of the plurality of ligation templates hybridized thereto may be the same type of link. In some cases, these links may be different types of links.

In some instances, the link may be formed with a cross-linker. The cross-linker may comprise a photolabile cross-linker. The cross-linker may comprise a photo-activated cross-linker. The cross-linker may comprise a photo-inactivated cross-linker. The cross-linker may comprise a photolabile cross-linker, a thermal cross-linker, a formal cross-linker, or a combination thereof. In some cases, a formal cross-linker may be a chemical cross-linker. In some instances, a cross-linker may comprise a homo-bifunctional cross-linker or a hetero-bifunctional crosslinker.

In some instances, the cross-linker may comprise a nucleoside. The cross-linker may comprise a modified nucleoside. The cross-linker may comprise a nucleoside analog. In some cases, the cross-linker may comprise a 3-Cyanovinylcarbazole nucleoside. In some cases, the cross-linker may comprise a 3-Cyanovinylcarbazole nucleoside with a 2′-deoxyribose backbone (^{CNV}K). In some cases, the cross-linker may comprise a 3-cyanovinylcarbazole with a D-threoninol backbone (^{CNV}D). In some cases, the cross-linker may comprise a pyranocarbazole nucleoside. In some cases, the cross-linker may comprise a pyranocarbazole nucleoside with a 2′-deoxyribose backbone (^{PC}X). In some cases, the cross-linker may comprise a pyranocarbazole nucleoside with a D-threoninol backbone (^{PCX}D). In some cases, the cross-linker may also comprise a deoxyribose or a ribose backbone. In some cases, the cross-linker may comprise a serinol backbone. In some cases, the cross-linker may comprise aldehyde, aryl azide, azido-2′-deoxyinosine, benzophenone, bromouracil, carmustine, cisplatin, chloro ethyl nitroso urea, click chemistry cross-linker, a Cyanovinylcarbazole nucleoside, coumarins, diazirine, disulfide linkage, halogenated nucleoside, methoxsalen, mitomycin C, nitrogen mustard, nitrous acid, a phenylselenide (PhSe) group, psoralen, a pyranocarbazole nucleoside, stilbene, thionucleoside, trioxsalen, or a combination thereof. In some cases, a thionucleoside may comprise a 4-Thio-dU-CE Phosphoramidite (i.e., 5′-Dimethoxytrityl-2′-deoxy-4-(2-cyanoethylthio)-Uridine,3′-[(2-cyanoethyl)-(N,N-diisopropyl)]-phosphoramidite), a 4-Thio-dT-CE Phosphoramidite (i.e., 5′-Dimethoxytrityl-2′-deoxy-4-(2-cyanoethylthio)-Thymidine,3′-[(2-cyanoethyl)-(N,N-diisopropyl)]-phosphoramidite), a 6-thio-dG-CE Phosphoramidite (i.e., 5′-Dimethoxytrityl-N2-trifluoroacetyl-2′-deoxy-6-(2-cyanoethyl)thio-Guanosine,3′-[(2-cyanoethyl)-(N,N-diisopropyl)]- phosphoramidite), or a combination thereof. In some cases, a click chemistry cross-linker may comprise an azide group or an alkyne group. In some cases, psoralen may comprise psoralen C2 or psoralen C6. In some cases, a diazirine cross-linker may comprise 2′-O-diazirine-conjugated adenosine ⁽DA). In some cases, an azido-2′-deoxyinosine cross-linker may comprise a 2-azido-2′-deoxyinosine and 8-azido-2′-deoxyadenosine. Other cross-linkers may comprise quinone methide, thienyl-substituted α-ketoamide, or tetrazole.

In some cases, a n^{th} polynucleotide (e.g., first polynucleotide, second polynucleotide, third polynucleotide, etc.) may comprise a cross-linker (e.g., a nucleotide comprising a cross-linker). After the n^{th} polynucleotide hybridizes with a ligation template, a cross-link may form between the cross-linker of the n^{th} polynucleotide and a nucleotide of the ligation template. In some cases, the nucleotide of the ligation template forming the cross-link may be in a −1 position relative to the cross-linker from 5′ to 3′ of the n^{th} polynucleotide (i.e., the nucleotide of the ligation template may be −1 nucleotide 5′ to the cross-linker, or the nucleotide comprising the cross-linker). In some cases, the nucleotide of the ligation template involved in the cross-link may be 1, 2, 3, 4, 5, or more nucleotides 5′ to the cross-linker or the nucleotide comprising the cross-linker of the n^{th} polynucleotide. In some cases, the nucleotide of the ligation template involved in the cross-link may be 1, 2, 3, 4, 5, or more nucleotides 3′ to the cross-linker or the nucleotide comprising the cross-linker of the n^{th} polynucleotide. The nucleotide of the ligation template forming the cross-link may comprise a cytosine (C), a thymine (T), a guanine (G), an adenosine (A), or a uracil (U). The nucleotide of the ligation template forming the cross-link may comprise a purine or a pyrimidine. The nucleotide of the ligation template forming the cross-link may comprise R, Y, S, W, K, M, B, D, H, V, or N according to the IUPAC nucleotide code. The nucleotide of the ligation template forming the cross-link may comprise a modified nucleotide or a nucleotide analog.

An n^{th} polynucleotide may comprise any nucleic acid, as described elsewhere herein. A ligation template may comprise any nucleic acid, as described elsewhere herein.

An n^{th} polynucleotide may comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 200, 500 or more nucleotides. Alternatively or in addition, an n^{th} polynucleotide may comprise at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 200, 500 or more nucleotides.

In some cases, an n^{th} polynucleotide may comprise a sequence complementary to a sequence in the ligation template, which may be configured to hybridize to the ligation template. The complementary sequence of the n^{th} polynucleotide may comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100 or more nucleotides. Alternatively or in addition, the complementary sequence of the n^{th} polynucleotide may comprise at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100 or more nucleotides. In some cases, the complementary sequence of the n^{th} polynucleotide may be disposed at one end of the n^{th} polynucleotide.

In some instances, an n^{th} polynucleotide may comprise one or more sequences useful for nucleic acid processing, such as a sequence for binding a nucleic acid, a sequence for hybridizing a nucleic acid, a sequence for binding a chemical moiety, a sequence for downstream sequencing, a primer sequence, an adapter sequence, a barcode sequence, a sequence acting as a linker, splint, or bridge, any complementary sequence thereof, or a combination thereof. For example, the n^{th} polynucleotide may comprise a priming sequence for binding to a primer for nucleic acid amplification. In another example, the n^{th} polynucleotide may comprise a sequence for binding an adaptor, such as a sequencing adaptor, flow cell adaptor, bead connecting adaptor, substrate connecting adaptor, or an amplification adaptor. In some cases, the one or more sequences useful for nucleic acid processing of the n^{th} polynucleotide may comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100 or more nucleotides. Alternatively or in addition, the one or more sequences useful for nucleic acid processing of the n^{th} polynucleotide may comprise at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100 or more nucleotides. An n^{th} polynucleotide may be a single-stranded nucleic acid, double-stranded nucleic acid, or partially double-stranded nucleic acid.

A ligation template may comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 200, 500 or more nucleotides. Alternatively or in addition, a ligation template may comprise at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 200, 500 or more nucleotides.

In some cases, the ligation template may comprise a sequence complementary to a sequence of an n^{th} polynucleotide. The ligation template may comprise multiple sequences complementary to a sequence of an n^{th} polynucleotide, such as one sequence disposed at each end of the ligation template. The multiple complementary sequences in the ligation template may be identical. Alternatively, the multiple complementary sequences in the ligation template may be different. In some cases, a complementary sequence of the ligation template may be common to multiple different n^{th} polynucleotides (e.g., common to first polynucleotides, second polynucleotides, and third polynucleotides, etc.). In some cases, the complementary sequence may be common to all n^{th} polynucleotides. In such cases, beneficially, the same ligation templates may be used for each round of polynucleotide linking. In some cases, a single ligation template may comprise multiple different types of complementary sequences (e.g., different sequences disposed at each end). Each complementary sequence on a single ligation template having multiple different types of complementary sequences may be common to different n^{th} polynucleotides (e.g., first complementary sequence may be common to first polynucleotides, second complementary sequence may be common to second polynucleotides, etc.). A complementary sequence of the ligation template may comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 200 or more nucleotides. Alternatively or in addition, the complementary sequence of the ligation template may comprise at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 200 or more nucleotides. The ligation template may be a single-stranded nucleic acid, double-stranded nucleic acid, or partially double-stranded nucleic acid.

In some instances, a ligation template may comprise one or more sequences useful for nucleic acid processing, such as a sequence for binding a nucleic acid, a sequence for hybridizing a nucleic acid, a sequence for binding a chemical moiety, a sequence for downstream sequencing, a primer sequence, an adapter sequence, a barcode sequence, a sequence acting as a linker, splint, or bridge, any complementary sequence thereof, or a combination thereof. In some cases, the one or more sequences useful for nucleic acid processing of the ligation template may comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100 or more nucleotides. In some cases, the one or more sequences useful for nucleic acid processing of the ligation template may comprise at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100 or more nucleotides. In some instances, the one or more sequences useful for nucleic acid processing of an n^{th} polynucleotide and the one or more sequences useful for nucleic acid processing of the ligation template may be the same or substantially the same. In other cases, the respective sequences useful for nucleic acid processing may be different.

In some instances, the ligation template may hybridize to the first polynucleotide and the second polynucleotide at least substantially simultaneously. In some cases, the ligation template may hybridize to the first polynucleotide prior to, during, or subsequent to the ligation template hybridizing to the second polynucleotide.

In some instances, an analyte may be coupled to a first polynucleotide. In some instances, multiple analytes may be coupled to a first polynucleotides. In some instances, multiple first polynucleotides may be coupled to an analyte. The first polynucleotide may bind, link, associate, or couple to the analyte. In some cases, the first polynucleotide may comprise a binding moiety to bind, link, associate, or couple to the analyte, and/or the analyte may comprise a binding moiety to bind, link, associate, or couple to the first polynucleotide. In some cases, the binding moiety may comprise a nucleic acid, an amino acid, a peptide, a saccharide, a polysaccharide, a protein, an antibody or antigen binding fragment thereof, an inorganic chemical compound, an organic chemical compound, or a combination thereof. In some instances, the coupling may comprise a covalent bond, non-covalent bond, or a combination thereof. In some cases, non-covalent bond of the coupling may comprise coordination bonding, hydrogen bonding, ionic interaction, van der Waals interaction, or a combination thereof.

In some instances, an n^{th} polynucleotide may comprise a barcode. In some cases, the barcode alone may be a spatial barcode that can identify an analyte or a plurality of analytes with a characteristic or property encoded by the spatial barcode (e.g., a type of analyte, a spatial location of the substrate, a sample origin, etc.). In some cases, a barcode of an n^{th} polynucleotide may combine with at least one other barcode of another n^{th} polynucleotide, where n is different, to generate a spatial barcode that identifies an analyte or a plurality of analytes with a characteristic or property encoded by the spatial barcode. A spatial barcode may comprise any number of barcodes from respective n^{th} polynucleotides, such as 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100 or more barcodes from respective n^{th} polynucleotides.

A barcode of an n^{th} polynucleotide may comprise a polynucleotide sequence. In some cases, a barcode may comprise a randomized sequence. In some cases, a barcode may comprise a pre-determined sequence which identity is known. In some cases, the barcode may comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100 or more nucleotides. Alternatively or in addition, the barcode may comprise at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100 or more nucleotides. A spatial barcode may be made up of barcodes that have the same sequences, different sequences, or a combination thereof in any order. A spatial barcode may be made up of barcodes that have the same lengths (e.g., of nucleotides), different lengths, or a combination thereof in any order. In some cases, the spatial barcode may comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100 or more nucleotides. Alternatively or in addition, the spatial barcode may comprise at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100 or more nucleotides.

Selective illumination under sufficient conditions may (i) link and/or cross-link a subset of a plurality of the first polynucleotides at the subset of the plurality of the individually addressable locations with a subset of a plurality of ligation templates hybridized thereto and/or (ii) link and/or cross-link at least a subset of the second polynucleotides with the subset of the plurality of the ligation templates hybridized thereto. More generally, after a plurality of n^{th} polynucleotides are provided to the substrate, selective illumination may (i) link and/or cross-link a subset of a plurality of the (n−1)^{th} polynucleotides at the subset of the plurality of the individually addressable locations with a subset of a plurality of ligation templates hybridized thereto and/or (ii) link and/or cross-link at least a subset of the n^{th} polynucleotides with the subset of the plurality of the ligation templates hybridized thereto.

In some instances, the selective illumination may comprise visible light. In some cases, the selective illumination may comprise ultraviolet (UV) light. In some cases, the wavelength of the light of the selective illumination may comprise at least about 300, 310, 320, 330, 340, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900 nanometers (nm) or more. In some cases, the wavelength of the light of the selective illumination may comprise at most about 300, 310, 320, 330, 340, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900 nm or more. In some cases, the wavelength of the light of the selective illumination may comprise at least about 10⁻³, 10⁻², 10⁻¹, 10¹, 10², 10³ nm or more. In some cases, the wavelength of the light of the selective illumination may comprise at most about 10⁻³, 10⁻², 10⁻¹, 10¹, 10², 10³ nm or more. In some cases, the selective illumination may comprise gamma light, x-ray light, UV light, infrared light, or a combination thereof. It will be appreciated that the light for the selective illumination may be selected based on the linking and/or cross-linking reagents involved between the n^{th} polynucleotides and the ligation templates.

In some instances, the selective illumination may span at least about 0.0001, 0.001, 0.01, 0.1, 1, 2, 3, 4, 5, 6, 7, 8, 9, 10, 20, 30, 40, 50, 60, 70, 80, 90, 100, 200, 300, 400, 500, 600, 700, 800, 900, 1000, 1100, 1200, 1300, 1400, 1500, 1600, 1700, 1800 seconds (s) or more. Alternatively or in addition, the selective illumination may span at most about 0.0001, 0.001, 0.01, 0.1, 1, 2, 3, 4, 5, 6, 7, 8, 9, 10, 20, 30, 40, 50, 60, 70, 80, 90, 100, 200, 300, 400, 500, 600, 700, 800, 900, 1000, 1100, 1200, 1300, 1400, 1500, 1600, 1700, 1800 s or more. It will be appreciated that duration of the selective illumination may be selected based on the linking and/or cross-linking reagents involved between the n^{th} polynucleotides and the ligation templates.

In some instances, the selection of the one or more selective regions receiving the selective illumination, corresponding to the subset of individually addressable locations of the substrate may be pre-determined or recorded (e.g., a pre-determined spatial location of the substrate). In some instances, an order or pattern of the selection of one or more selective regions with each cycle may be pre-determined or recorded. The individually addressable locations of the substrate may be selectively illuminated by an illumination system. The illumination system may comprise a light source (e.g., LED light source, UV light source, IR light source, laser light source, etc.) configured to provide a light of one or more of any wavelengths described herein. The illumination system may comprise an array of mirrors or micro-mirrors. In some cases, the illumination system may comprise multifaceted mirrors or micro-mirrors. The array of mirrors, array of micro-mirrors, multifaceted mirrors, or multifaceted micro-mirrors may facilitate selective illumination on the substrate. The array of mirrors, array of micro-mirrors, multifaceted mirrors, or multifaceted micro-mirrors may provide a resolution of the selective illumination when the light is illuminating the substrate. In some cases, the array of mirrors, array of micro-mirrors, multifaceted mirrors, or multifaceted micro-mirrors may comprise or be a part of a Digital Micromirror Device (DMD) or other optical device. For example, the illumination system may comprise a digital micromirror device or other optical device. In some cases, each or a subset of the array of mirrors, the array of the micromirrors, the multifaceted mirrors, or the multifaceted micromirrors may have a state, such as an ON and/or an OFF state. In some cases, when it is in the ON state, a mirror or micromirror, or subset of mirrors or micromirrors, may facilitate the illumination of a particular spatial location of the substrate. In some cases, when it is in the ON state, a mirror or micromirror, or subset of mirrors or micromirrors, may block the illumination of a particular spatial location of the substrate. In some cases, when it is in the OFF state, a mirror or micromirror, or subset of mirrors or micromirrors, may block the illumination of a particular spatial location of the substrate. In some cases, when it is in the OFF state, a mirror or micromirror, or subset of mirrors or micromirrors, may facilitate the illumination of a spatial location of the substrate.

In some instances, the one or more selective regions of a substrate illuminated by selective illumination may be addressable by the combinations of ON/OFF states of the mirrors or micromirrors. For example, a mirror or micromirror may be configured to facilitate the selective illumination in its ON state. To allow for selective illumination, the mirrors or micromirrors that facilitate the selective illumination at the one or more selective regions of the substrate may be maintained or directed to their ON state, while the other mirrors or micromirrors are maintained or directed to their OFF state. Analogous arrangements may be applied if a mirror or micromirror is configured to facilitate the selective illumination at the substrate in its OFF state. In some cases, the control of the ON and/or OFF state of a mirror or micromirror may be facilitated by the computer systems described herein. In some cases, the resolution may comprise at least about 10², 10³, 10⁴, 10⁵, 10⁶, 10⁷, 10⁸, 10⁹, 10¹⁰ pixels or more. In some cases, the resolution may comprise at most about 10², 10³, 10⁴, 10⁵, 10⁶, 10⁷, 10⁸, 10⁹, 10¹⁰ pixels or more. In some cases, the resolution may also comprise at least about 640×480, 1280×720, 1920×1080, 2560×1440, 2048×1080, 3840×2160, or 7680×4320 pixels. In some cases, the resolution may also comprise at most about 640×480, 1280×720, 1920×1080, 2560×1440, 2048×1080, 3840×2160, or 7680×4320 pixels.

In some instances, a pitch size of the pixel may be at least about 0.01, 0.1, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10 micrometers (μm) or more. In some cases, a pitch size of the pixel may be at most about 0.01, 0.1, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10 micrometers (μm) or more. In some cases, a pitch size of the pixel may be the same or substantially the same as the pitch size of an individually addressable location. In some cases, the pitch size of the pixel is at least within about 1%, 5%, 10%, 15%, 20%, 30%, 40%, 50% or more of the pitch size of the individually addressable location. In some cases, the pitch size of the pixel is at most within about 1%, 5%, 10%, 15%, 20%, 30%, 40%, 50% or more of the pitch size of the individually addressable location.

The method may further comprise, subsequent to subjecting the substrate with the selective illumination after providing or applying a plurality of n^{th} polynucleotides to the substrate, removing a plurality of non-linked n^{th} polynucleotides and/or a plurality of non-linked ligation templates, such as from the non-selected regions and/or from the selected regions, from the substrate. In some cases, the removing may comprise lowering a melting temperature of the bond(s) between the (n−1)^{th} polynucleotide, the n^{th} polynucleotide, the ligation template, or a combination thereof. In some cases, the removing may comprise providing a solution. The solution may lower the melting temperature of the bond(s) between the (n−1)^{th} polynucleotide, the n^{th} polynucleotide, the ligation template, or a combination thereof. Upon contact with the solution, any complexes between the plurality of (n−1)^{th} polynucleotides, the plurality of nth polynucleotides, and the plurality of ligation templates that are not otherwise linked (via the selective illumination) may dissolve to release the n^{th} polynucleotides and ligation templates as hybridizations reverse. The substrate may be subjected to a washing buffer or washing solution to retain the immobilized polynucleotides and remove any non-immobilized polynucleotides (e.g., free floating n^{th} polynucleotides and ligation templates) from the substrate. In some cases, the solution to lower the melting temperature may comprise dimethyl sulfoxide (DMSO). In some cases, DMSO may be least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, 99% or more by volume in the solution. In some cases, DMSO may be most about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, 99% or more by volume in the solution. The washing solution may comprise other washing reagents, such as sodium hydroxide (NaOH), formamide, and the like. In some cases, providing the washing solution to remove non-linked second polynucleotide or a non-linked ligation template from the substrate may span for at least about 0.0001, 0.001, 0.01, 0.1, 1, 2, 3, 4, 5, 6, 7, 8, 9, 10, 20, 30, 40, 50, 60, 70, 80, 90, 100, 110, 120, 130, 140, 150, 200, 300 s or more. In some cases, providing the washing solution to remove non-linked second polynucleotide or a non-linked ligation template from the substrate may span for at most about 0.0001, 0.001, 0.01, 0.1, 1, 2, 3, 4, 5, 6, 7, 8, 9, 10, 20, 30, 40, 50, 60, 70, 80, 90, 100, 110, 120, 130, 140, 150, 200, 300 s or more. In some instances, the solution may be provided to the substrate at least 1, 2, 3, 4, 5 or more times. In some instances, the solution may be provided to the substrate at most 1, 2, 3, 4, 5 or more times. Alternatively or in addition, the solution may comprise magnesium chloride. Alternatively or in addition, the removing may also comprise increasing the temperature of the reaction space, the substrate, the first polynucleotide, the second polynucleotide, the ligation template, or a combination thereof. Alternatively or in addition, the removing comprises applying one or more other stimuli.

Then, the solution may be removed, such as by washing the substrate. In some cases, the washing may comprise a washing solution. In some cases, the washing solution may comprise water or a buffer. In some cases, the washing solution may comprise Phosphate-buffered saline (PBS). In some cases, the PBS may have a concentration of at least about 1 nanomolar (nM), 1 micromolar (μM), 1 millimolar (mM), 1 molar (M), 10 M or more. In some cases, the PBS may have a concentration of at most about 1 nanomolar (nM), 1 micromolar (μM), 1 millimolar (mM), 1 molar (M), 10 M or more. In some cases, the removing may comprise at least 1, 2, 3, 4, 5, 6, 7, 8, 9 10 or more cycles of washing. In some cases, the removing may comprise at most 1, 2, 3, 4, 5, 6, 7, 8, 9 10 or more cycles of washing. In some cases, the washing may span at least about 0.001, 0.01, 0.1, 1, 2, 3, 4, 5, 6, 7, 8, 9, 10, 20, 30, 40, 50, 60, 70, 80, 90, 100, 110, 120, 130, 140, 150, 200, 300 seconds (s) or more. In some cases, the washing may span at most about 0.001, 0.01, 0.1, 1, 2, 3, 4, 5, 6, 7, 8, 9, 10, 20, 30, 40, 50, 60, 70, 80, 90, 100, 110, 120, 130, 140, 150, 200, 300 seconds (s) or more.

In some instances, the method may further comprise, upon or after formation of the links of the subset of the plurality of first polynucleotides at the subset of individually addressable locations and the subset of the plurality of ligation templates hybridized thereto and/or formation of the links of the subset of the plurality of second polynucleotides and the subset of the plurality of ligation templates hybridized thereto, subjecting the subset of the plurality of first polynucleotides and the subset of the plurality of second polynucleotides to a second condition sufficient for the subset of the plurality of first polynucleotides at the individually addressable locations and the subset of the plurality of second polynucleotides to form a bond. Generally, the method may further comprise, upon or after formation of the links of the subset of the plurality of (n−1)^{th} polynucleotides at the subset of the plurality of individually addressable locations and the subset of the plurality of ligation templates hybridized thereto and/or a formation of the links of the subset of plurality of n^{th} polynucleotides and the subset of plurality of ligation templates hybridized thereto, subjecting the substrate to a second condition sufficient for the subset of the plurality of (n−1)^{th} polynucleotides at the subset of individually addressable locations and the subset of the plurality of n^{th} polynucleotides to form a bond. The formation of the bonds may also occur prior to or during the formation of the links of the subset of the plurality of first polynucleotides at the subset of individually addressable locations and the subset of the plurality of ligation templates hybridized thereto and/or a formation of the links of the subset of the plurality of second polynucleotides and the subset of the plurality of ligation templates hybridized thereto.

In some instances, the bond between the (n−1)^{th} polynucleotide at the selected individually addressable location and the n^{th} polynucleotide may be formed by a ligase. In some cases, the second condition may comprise conditions sufficient for a ligation reaction to occur between the (n−1)^{th} polynucleotide at the selected individually addressable location and the n^{th} polynucleotide. In some cases, the bond between the (n−1)^{th} polynucleotide at the selected individually addressable location and the n^{th} polynucleotide may comprise a phosphodiester bond. In other cases, the bond between the (n−1)^{th} polynucleotide at the selected individually addressable location and the n^{th} polynucleotide may comprise a covalent or non-covalent bond described elsewhere in this disclosure.

In some instances, the method may comprise, subsequent to the formation of the bonds between the subset of the plurality of (n−1)^{th} polynucleotides at the subset of plurality of individually addressable locations and the subset of the plurality of n^{th} polynucleotides, performing an amplification reaction to generate a plurality of amplification products of the subset of the plurality of (n−1)^{th} polynucleotides coupled to the subset of the plurality of n^{th} polynucleotides. In some cases, the amplification reaction may be performed prior to or during the formation of the bonds between the subset of the plurality of (n−1)^{th} polynucleotides at the subset of plurality of individually addressable locations and the subset of the plurality of n^{th} polynucleotides. In some cases, the amplification reaction may generate a double stranded nucleic acid amplification products. In some cases, the amplification reaction may generate a complementary DNA (cDNA) amplification products.

The amplification reaction may comprise a nucleic acid amplification reaction. In some cases, the nucleic acid amplification may comprise a polymerase chain reaction (PCR). In some cases, the nucleic acid amplification may comprise an asymmetric amplification reaction, a helicase-dependent amplification (HDA), a ligase chain reaction (LCR), a loop mediated isothermal amplification (LAMP), a multiple displacement amplification (MDA), a nucleic acid sequence based amplification (NASBA), a PCR, a primer extension, a recombinase polymerase amplification (RPA), a rolling circle amplification (RCA), a self-sustained sequence replication (3SR), a strand displacement amplification (SDA), a reverse transcription, or a combination thereof. In some instances, the amplification reaction may generate a plurality of amplification products of the (n−1)^{th} polynucleotide and the n^{th} polynucleotide that have formed the bond. The amplification reaction may be performed on or off the substrate. In some cases, the amplification products may not be attached or immobilized to the substrate. In other cases, the amplification products may be attached or immobilized to the substrate. In some cases, the amplification products may be attached or immobilized to one or more individually addressable locations of the substrate. In some cases, the amplification products may be attached or immobilized to an object (e.g., bead) that is immobilized to one or more individually addressable locations of the substrate.

In some instances, the method may further comprise subjecting the plurality of (n−1)^{th} polynucleotides, the plurality of n^{th} polynucleotides, and/or the plurality of ligation templates to a second light illumination subsequent to subjecting the substrate with the selective illumination.

In some instances, the link or cross-link, once formed, may be reversible. In some cases, the link or cross-link, once formed, may be irreversible.

In some cases, the second illumination may break or facilitate to break the link or cross-link between: (i) the subset of the plurality of (n−1)^{th} polynucleotides immobilized at the substrate and the subset of the plurality of ligation templates hybridized thereto, or (ii) the subset of the plurality of n^{th} polynucleotides and the subset of the plurality of ligation templates hybridized thereto. In some cases, the second illumination may break or facilitate to break the link or cross-link between: (i) the subset of the plurality of (n−1)^{th} polynucleotides immobilized at the substrate and the subset of the plurality of ligation templates hybridized thereto, and (ii) the subset of the plurality of n^{th} polynucleotides and the subset of the plurality of ligation templates hybridized thereto.

In some instances, the second illumination may comprise visible light. In some cases, the second illumination may comprise ultraviolet (UV) light. In some cases, the wavelength of the light of the second illumination may comprise at least about 250, 260, 270, 280, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 340, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900 nm or more. In some cases, the wavelength of the light of the second illumination may comprise at most about 250, 260, 270, 280, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 340, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900 nm or more. In some cases, the wavelength of the light of the second illumination may comprise at least about 10⁻³, 10⁻², 10⁻¹, 10¹, 10², 10³ nm or more. In some cases, the wavelength of the light of the second illumination may comprise at most about 10⁻³, 10⁻², 10⁻¹, 10¹, 10², 10³ nm or more. In some cases, the second illumination may comprise gamma light, x-ray light, UV light, infrared light, or a combination thereof. The second illumination may have a different wavelength than the first illumination. It will be appreciated that the light for the second illumination may be selected based on the linking and/or cross-linking reagents involved between the n^{th} polynucleotides and the ligation templates.

In some instances, the second illumination may span at least about 0.0001, 0.001, 0.01, 0.1, 1, 2, 3, 4, 5, 6, 7, 8, 9, 10, 20, 30, 40, 50, 60, 70, 80, 90, 100, 200, 300, 400, 500, 600 seconds (s) or more. In some cases, the second illumination may span at least about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 30, 40, 50, 60 minutes or more. In some cases, the second illumination may span at most about 0.0001, 0.001, 0.01, 0.1, 1, 2, 3, 4, 5, 6, 7, 8, 9, 10, 20, 30, 40, 50, 60, 70, 80, 90, 100, 200, 300, 400, 500, 600 seconds (s) or more. In some cases, the second illumination may span at most about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 30, 40, 50, 60 minutes or more. It will be appreciated that the duration for the second illumination may be selected based on the linking and/or cross-linking reagents involved between the n^{th} polynucleotides and the ligation templates.

In some instances, the analyte may comprise a tissue, a cell, a nucleic acid, a protein, a lipid, a saccharide, a polysaccharide, any sample described herein, or a combination thereof. The nucleic acid may comprise any nucleic acid described herein. The protein may comprise an intracellular protein and/or an extracellular protein. In some cases, the extracellular protein may comprise a cell surface protein. In some cases, the extracellular protein may comprise a cell membrane protein. In some cases, the cell membrane protein may comprise a type I or type II cell membrane protein. In other cases, the cell surface protein may comprise a glycosylphosphatidylinositol (GPI) anchor or modification. In some cases, the cell may be a eukaryotic cell or a prokaryotic cell. In some cases, the prokaryotic cell may comprise a bacterial cell. In some cases, the eukaryotic cell may comprise a mammalian cell. In some cases, the mammalian cell may comprise a human cell. In some cases, the mammalian cell may comprise a mouse cell, a hamster cell, a rodent cell, a rat cell, a rabbit cell, a pig, a guinea pig, a camel, or a combination thereof. In some cases, the eukaryotic cell may comprise an insect cell, a fish cell, an amphibian cell, a reptile cell, a mammalian cell, or a combination thereof. In some cases, a eukaryotic cell may comprise a fly cell, a frog cell, a zebrafish cell, a yeast cell, a nematode cell, a planarian cell, or a combination thereof. The spatial barcodes may tag multiple different types of analytes.

In some instances, the cell may be fixed. In some cases, the cell may be permeabilized. In some cases, the cell may comprise the protein. In some cases, the analyte-binding moiety of the first polynucleotide may contact and/or bind to the analyte of the fixed or permeabilized cell. For example, an intracellular analyte, such as an intracellular protein, nucleic acid, protein, lipid, saccharide, polysaccharide, or a combination thereof, in a fixed or permeabilized cell may be contacted, bound, and/or coupled by the analyte-binding moiety of the first polynucleotide.

In some instances, the method may comprise (a) providing the substrate, wherein the substrate comprises a plurality of individually addressable locations comprising a plurality of the first polynucleotides immobilized thereto, optionally wherein a plurality of analytes are immobilized to the plurality of individually addressable locations and the plurality of first polynucleotides are coupled to the plurality of analytes; (b) providing or applying to the substrate, e.g., in a first fluid layer, (i) a plurality of the second polynucleotides comprising a first barcode sequence, and (ii) a first plurality of the ligation templates; (c) subjecting a first subset of the plurality of the individually addressable locations to selective illumination, under conditions sufficient to: (i) link at least a subset of the plurality of the first polynucleotides at the first subset of the plurality of the individually addressable locations with at least a subset of the first plurality of the ligation templates hybridized thereto, and (ii) link at least a subset of the plurality of the second polynucleotides with at least the subset of the first plurality of the ligation templates hybridized thereto; (d) providing or applying to the substrate, e.g., in a second fluid layer, (i) a plurality of third polynucleotides comprising a second barcode sequence, and (ii) a second plurality of ligation templates; (c) subjecting a second subset of the plurality of individually addressable locations to the selective illumination, under conditions sufficient to: (i) link a second subset of the plurality of second polynucleotides at the second subset of the plurality of the individually addressable locations with a subset of the second plurality of the ligation templates hybridized thereto, and (ii) link at least a subset of the plurality of the third polynucleotides with the subset of the second plurality of the ligation templates hybridized thereto.

In some instances, the first fluid layer has a thickness of at most 50 micrometers. In some instances, the second fluid layer has a thickness of at most 15 micrometers.

In some instances, the first barcode sequence and the second barcode sequence may comprise the same or substantially the same sequence. In some cases, the first barcode sequence and the second barcode sequence may comprise different sequences. In some cases, first barcode sequence and the second barcode sequence may comprise any features of the barcode sequences described herein. In some cases, the third polynucleotide may comprise any features of the polynucleotides described herein.

In some instances, the first plurality of ligation templates and the second plurality of ligation templates may comprise the same or substantially the same sequence. In other cases, the first plurality of ligation templates and the second plurality of ligation templates may comprise different sequences. In some cases, the first subset and the second subset of the plurality of the individually addressable locations may comprise the same or substantially the same individually addressable locations. In some cases, the first subset and the second subset of the plurality of the individually addressable locations may comprise a common subset of the individually addressable locations. In some cases, the common subset of the individually addressable locations may comprise at least about 10%, 20%, 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, 98%, 99% or more of the first subset and/or the second subset of the plurality of the individually addressable locations. In some cases, the common subset of the individually addressable locations may comprise at most about 10%, 20%, 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, 98%, 99% or more of the first subset and/or the second subset of the plurality of the individually addressable locations. In some cases, the first subset and the second subset of the plurality of the individually addressable locations may comprise different individually addressable locations. In some cases, the first subset and the second subset of the plurality of the individually addressable locations may comprise a non-overlapping subset of the individually addressable locations. In some cases, the first subset and the second subset of the plurality of the individually addressable locations may be based on a pre-determined spatial location of the substrate. In some cases, the first subset and the second subset of the plurality of the individually addressable locations may be based on a randomized spatial location of the substrate.

In some instances, the methods may further comprise: (1) subjecting the plurality of the first polynucleotides, the plurality of the second polynucleotides, and/or the plurality of the ligation templates to a second light illumination subsequent to subjecting the substrate with selective illumination; (2) removing the non-linked second polynucleotides or the non-linked ligation templates; (3) subjecting the first polynucleotide and/or the second polynucleotide to the second condition; (4) performing the amplification reaction; or (5) any combination thereof in any order subsequent to the subjecting of (c) or (d). In other cases, steps (1), (2), (3), (4), or (5) may be carried out prior to or during the providing of (d) and/or the subjecting of (e).

In some instances, the method may comprise repeating the providing of (d) and/or the subjecting of (e) at least once. In some instances, the method may comprise repeating the providing of (d) and/or the subjecting of (e) at least 1, 2, 3, 4, 5, 6, 7, 8, 9, 10 or more times. In some instances, the method may comprise repeating the providing of (d) and/or the subjecting of (e) at most once. In some instances, the method may comprise repeating the providing of (d) and/or the subjecting of (e) at most 1, 2, 3, 4, 5, 6, 7, 8, 9, 10 or more times. A different subset or the same subset of individually addressable locations may be selected during one or more repeats. In some cases, after concluding all the repeat(s), the final spatial barcodes immobilized to the substrate may each have a same length of nucleotides, or some or all spatial barcodes may have different lengths of nucleotides depending on how many times a certain individually addressable location was subjected to selective illumination.

In some instances, repeating the providing or applying of (d) and/or the subjecting of (e) may increase the number of unique spatial barcodes. In some cases, the increase of the number of unique spatial barcodes produced by the method, with X cycles of illuminations, may be calculated by the formula: N^{X}, where a single cycle includes N number of repeats to cover N number of subsets of individually addressable locations of a substrate, where N number of unique barcode sequences are provided in each cycle of repeating (d) and (e), including the cycle of (b) and (c), wherein the cycle (b) and (c) may be the cycle of (d) and (e), respectively. For example, 4 cycles of repeating (d) and (e) (including (b) and (c)) with each cycle comprising 32 different unique barcode sequences may produce 32⁴=1,048,576 different unique barcode sequences. In other cases, the increase of the number of unique spatial barcodes produced by the method may be calculated by multiplying the number of unique barcode sequences provided for each cycle (e.g., if 5 unique barcode sequences are provided in the 1^{st} cycle, 8 unique barcode sequences are provided in the 2^{nd} cycle, and 21 unique barcode sequences are provided in the 3^{rd} cycle, there will be an estimated 5*8*21=840 unique barcode sequences generated).

The provided systems, methods, kits, and compositions allow for the generation of unique spatial barcodes in pre-determined spatial locations of a substrate, directed by iterative cycles of selective illumination on the substrate. In some cases, the spatial barcodes are generated on a surface of the substrate at such spatial locations, such as to provide a barcode-encoded substrate surface. In some cases, the identities and locations of the spatial barcodes with respect to the substrate may be known. Downstream, the surface may subsequently receive a sample comprising one or more analytes such that the spatial barcodes can contact and tag the analytes. The spatial barcodes may be released from the surface subsequent to contacting and/or tagging the analytes. In other cases, the spatial barcodes may be released from the surface prior to contacting and/or tagging the analytes, such as to diffuse into a sample (e.g., tissue sample) to tag an analyte in the sample. Alternatively or in addition, the generated spatial barcodes may be released from the surface, collected, and contact and tag one or more analytes off the substrate, such as in another reaction environment. In other cases, the spatial locations on the substrate may have immobilized thereto one or more analytes, and the spatial barcodes may be generated directly on the one or more analytes at such spatial locations. Beneficially, a diverse set of barcodes may be generated without the need to perform physical split and pool operations. Beneficially, the identities and locations of the final barcodes generated may be known. Beneficially, in cases where the final spatial barcodes are generated directly on the analytes, a separate tagging step may be unneeded. In some cases, the sequence identity and the spatial location of a spatial barcode can be determined based on the cycles of selective illumination, the sequence identity of the different sets of unique sequences, and/or the spatial location of the selective illumination.

It will be appreciated that while the systems, methods, kits, and compositions provided herein describe the combinatorial generation of spatial barcodes, wherein a final barcode sequence is generated by combining individual barcode segments, any useful sequence may be flexibly generated by designing sequence segments and adding them in an appropriate order. For example, the systems, methods, kits, and compositions described herein may be used to generate a probe array, where a probing or capture sequence is added during one or more cycles. Such probe array may or may not include a barcode sequence. For example, the systems, methods, kits, and compositions described herein may be used to generate a final sequence that comprises any sequence useful for any nucleic acid processing operation, as described elsewhere herein. In addition, a location of a useful sequence segment (e.g., primer sequence) may be focused by adding the useful sequence segment at an appropriate order.

The present disclosure provides systems, compositions, and kits that can carry out or facilitate the methods described thereof. The systems, compositions, and kits may comprise any one of the first polynucleotides; the second polynucleotides; set(s) of n^{th} polynucleotides; the substrates; the analytes; the solutions; the linkers or the cross-linkers; the washing solutions; the arrays of mirrors or micromirrors; the multifaceted mirrors or micromirrors; a portion thereof; or a combination thereof. In some cases, the systems, compositions, and kits may comprise the reagents for the nucleic acid processing described elsewhere in this disclosure.

### Computer Systems

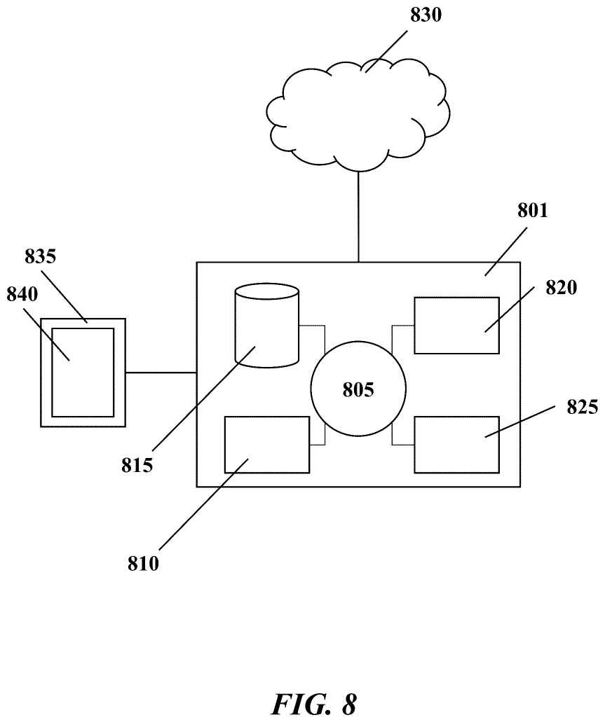

The present disclosure provides computer control systems that are programmed to implement methods of the disclosure. FIG. 8 shows a computer system 801 that is programmed or otherwise configured to implement methods of the disclosure, such as to control the systems described herein (e.g., reagent dispensing, detecting, illuminating, etc.) and/or collect, receive, and/or analyze sequencing information. The computer system 801 may be configured to regulate, for example, a DMD device. The computer system 801 can be an electronic device of a user or a computer system that is remotely located with respect to the electronic device. The electronic device can be a mobile electronic device.

The computer system 801 includes a central processing unit (CPU, also “processor” and “computer processor” herein) 805, which can be a single core or multi core processor, or a plurality of processors for parallel processing. The computer system 801 also includes memory or memory location 810 (e.g., random-access memory, read-only memory, flash memory), electronic storage unit 815 (e.g., hard disk), communication interface 820 (e.g., network adapter) for communicating with one or more other systems, and peripheral devices 825, such as cache, other memory, data storage and/or electronic display adapters. The memory 810, storage unit 815, interface 820 and peripheral devices 825 are in communication with the CPU 805 through a communication bus (solid lines), such as a motherboard. The storage unit 815 can be a data storage unit (or data repository) for storing data. The computer system 801 can be operatively coupled to a computer network (“network”) 830 with the aid of the communication interface 820. The network 830 can be the Internet, an internet and/or extranet, or an intranet and/or extranet that is in communication with the Internet. The network 830 in some cases is a telecommunication and/or data network. The network 830 can include one or more computer servers, which can enable distributed computing, such as cloud computing. The network 830, in some cases with the aid of the computer system 801, can implement a peer-to-peer network, which may enable devices coupled to the computer system 801 to behave as a client or a server.

The CPU 805 can execute a sequence of machine-readable instructions, which can be embodied in a program or software. The instructions may be stored in a memory location, such as the memory 810. The instructions can be directed to the CPU 805, which can subsequently program or otherwise configure the CPU 805 to implement methods of the present disclosure. Examples of operations performed by the CPU 805 can include fetch, decode, execute, and writeback. In some cases, the instructions may direct any or a subset of the mirrors or micromirrors to their ON/OFF states. In some cases, the instructions may maintain any or a subset of the mirrors or micromirrors in their ON/OFF states. In some cases, the instructions may switch any or a subset of the mirrors or micromirrors between their ON and OFF states. In some instances, the instructions may modify the positioning (e.g., change an angle) of any or a subset of the mirrors or micromirrors. In some instances, the instructions may maintain the position of any or a subset of the mirrors or micromirrors.

The CPU 805 can be part of a circuit, such as an integrated circuit. One or more other components of the system 801 can be included in the circuit. In some cases, the circuit is an application specific integrated circuit (ASIC).

The storage unit 815 can store files, such as drivers, libraries, and saved programs. The storage unit 815 can store user data, e.g., user preferences and user programs. The computer system 801 in some cases can include one or more additional data storage units that are external to the computer system 801, such as located on a remote server that is in communication with the computer system 801 through an intranet or the Internet.

The computer system 801 can communicate with one or more remote computer systems through the network 830. For instance, the computer system 801 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 801 via the network 830.

Methods as described herein can be implemented by way of machine (e.g., computer processor) executable code stored on an electronic storage location of the computer system 801, such as, for example, on the memory 810 or electronic storage unit 815. The machine executable or machine-readable code can be provided in the form of software. During use, the code can be executed by the processor 805. In some cases, the code can be retrieved from the storage unit 815 and stored on the memory 810 for ready access by the processor 805. In some situations, the electronic storage unit 815 can be precluded, and machine-executable instructions are stored on memory 810.

The code can be pre-compiled and configured for use with a machine having a processor adapted to execute the code or can be compiled during runtime. The code can be supplied in a programming language that can be selected to enable the code to execute in a pre-compiled or as-compiled fashion.

Aspects of the systems and methods provided herein, such as the computer system 801, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 801 can include or be in communication with an electronic display 835 that comprises a user interface (UI) 840 for providing (e.g., displaying), for example, results of a nucleic acid sequence (e.g., sequence reads). In another example, the spatial location of the substrate with selective illumination or the mirrors or micromirrors that allows for the selective illumination at the spatial location may be displayed by the computer system. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 805. The algorithm can, for example, spatially resolve a plurality of analyte sequences using sequencing information.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### NUMBERED EMBODIMENTS

The following embodiments recite non-limiting permutations of combinations of features disclosed herein. Other permutations of combinations of features are also contemplated. In particular, each of these numbered embodiments is contemplated as depending from or relating to every previous or subsequent numbered embodiment, independent of their order as listed.
Embodiment 1. A method, comprising: (a) providing a substrate, wherein said substrate comprises a plurality of individually addressable locations comprising a plurality of first polynucleotides immobilized thereto; (b) applying a fluid layer to said substrate, wherein said fluid layer comprises (i) a plurality of second polynucleotides and (ii) a plurality of ligation templates, under conditions sufficient to hybridize said plurality of first polynucleotides to said plurality of ligation templates and to hybridize said plurality of second polynucleotides to said plurality of ligation templates, wherein said fluid layer has a thickness of at most 50 micrometers; and (c) subsequent to hybridization in (b), selectively illuminating a subset of said plurality of individually addressable locations, under conditions sufficient to: (i) link a subset of said plurality of first polynucleotides at said subset of said plurality of individually addressable locations with a subset of said plurality of ligation templates, and (ii) link a subset of said plurality of second polynucleotides with said subset of said plurality of ligation templates.
Embodiment 2. The method of embodiment 1, further comprising: (d) subsequent to (c), removing a plurality of non-linked second polynucleotides or a plurality of non-linked ligation templates from said substrate.
Embodiment 3. The method of embodiment 2, wherein said removing in (d) comprises providing to said substrate a solution comprising dimethyl sulfoxide (DMSO), sodium hydroxide (NaOH), or formamide.
Embodiment 4. The method of embodiment 3, wherein said solution comprises said DMSO at least about 1% by volume in said solution.
Embodiment 5. The method of any one of embodiments 3-4, further comprising, subsequent to (d), washing said substrate to remove said solution from said substrate.
Embodiment 6. The method of any one of embodiments 1-5, wherein said subset of said plurality of individually addressable locations is selected based on a pre-determined spatial location of said substrate corresponding to said subset of said plurality of individually addressable locations.
Embodiment 7. The method of embodiment 6, wherein (c) comprises using a Digital Micromirror Device (DMD) to address said pre-determined spatial location.
Embodiment 8. The method of any one of embodiments 1-7, further comprising, subsequent to linking in (c), (e) subjecting said substrate to conditions sufficient for said subset of said plurality of first polynucleotides and said subset of said plurality of second polynucleotides to form a bond.
Embodiment 9. The method of embodiment 8, wherein a ligase catalyzes coupling of said subset of said plurality of first polynucleotides and said subset of said plurality of second polynucleotides.
Embodiment 10. The method of embodiment 8 or 9, wherein, subsequent to (e), phosphodiester bonds are formed between said subset of said plurality of first polynucleotides and said subset of said plurality of second polynucleotides.
Embodiment 11. The method of any one of embodiments 8-10, further comprising, subsequent to (e), performing an amplification reaction to generate a plurality of amplification products of said subset of said plurality of first polynucleotides coupled to said subset of said plurality of second polynucleotides.
Embodiment 12. The method of embodiment 11, further comprising sequencing said plurality of amplification products, or derivatives thereof.
Embodiment 13. The method of any one of embodiments 1-12, wherein said selectively illuminating in (c) comprises providing ultraviolet (UV) light.
Embodiment 14. The method of embodiment 13, wherein said UV light comprises a wavelength of about 365 nanometers (nm).
Embodiment 15. The method of any one of embodiments 1-14, wherein said selectively illuminating in (c) comprises providing UV light for at most about 1 minute.
Embodiment 16. The method of any one of embodiments 1-15, further comprising, subsequent to said selective illumination in (c), subjecting said plurality of first polynucleotides, said plurality of second polynucleotides, and said plurality of ligation templates to an additional illumination, under conditions sufficient to break a subset of a plurality of links generated in (c) between (i) said subset of said plurality of first polynucleotides and said subset of said plurality of ligation templates, or (ii) said subset of said plurality of second polynucleotides and said subset of said plurality of ligation templates.
Embodiment 17. The method of embodiment 16, wherein said additional illumination comprises UV light.
Embodiment 18. The method of embodiment 17, wherein said UV light comprises a wavelength of about 312 nanometers (nm).
Embodiment 19. The method of any one of embodiments 1-18, wherein a plurality of analytes are immobilized to said plurality of individually addressable locations, and wherein said plurality of first polynucleotides are coupled to said plurality of analytes.
Embodiment 20. The method of embodiment 19, further comprising, prior to (a), (i) providing said substrate, (ii) immobilizing said plurality of analytes at said plurality of individually addressable locations, and (iii) coupling said plurality of first polynucleotides to said plurality of analytes.
Embodiment 21. The method of any one of embodiments 19-20, wherein said plurality of first polynucleotides is coupled to said plurality of analytes via a plurality of analyte-binding moieties of said plurality of first polynucleotides.
Embodiment 22. The method of embodiment 21, wherein said plurality of analyte-binding moieties comprises comprise one or more members selected from the group consisting of: nucleic acids, proteins, lipids, saccharides, polysaccharides, and a combination thereof.
Embodiment 23. The method of embodiment 22, wherein said proteins comprise antibodies or antigen binding fragments thereof.
Embodiment 24. The method of any one of embodiments 19-23, wherein said plurality of analytes comprises one or more members selected from the group consisting of: nucleic acids, proteins, cells, lipids, saccharides, polysaccharides, and a combination thereof.
Embodiment 25. The method of embodiment 24, wherein said proteins comprise one or more of extracellular proteins, cell surface proteins, cell membrane proteins, and intracellular proteins.
Embodiment 26. The method of any one of embodiments 1-18, wherein a plurality of beads are immobilized to said plurality of individually addressable locations, and wherein said plurality of first polynucleotides are coupled to said plurality of beads.
Embodiment 27. The method of any one of embodiments 1-26, wherein (c) comprises (i) cross-linking said subset of said plurality of first polynucleotides with said subset of said plurality of ligation templates, and (ii) cross-linking said subset of said plurality of second polynucleotides with said subset of said plurality of ligation templates.
Embodiment 28. The method of embodiment 27, wherein said plurality of first polynucleotides comprises a plurality of cross-linkers used in said cross-linking in (c)(i).
Embodiment 29. The method of embodiment 28, wherein said plurality of cross-linkers comprises 3-Cyanovinylcarbazole nucleosides (CNVKs).
Embodiment 30. The method of any one of embodiments 28 or 29, wherein said cross-linking in (c)(i) comprises forming a cross-link between a cross-linker of said plurality of cross-linkers and a nucleotide of said plurality of ligation templates.
Embodiment 31. The method of embodiment 30, wherein said nucleotide of said plurality of ligation templates is a cytosine (C) or a thymine (T).
Embodiment 32. The method of embodiment 31, wherein said nucleotide of said plurality of ligation templates is a C.
Embodiment 33. The method of embodiment 31, wherein said nucleotide of said plurality of ligation templates is a T.
Embodiment 34. The method of any one of embodiments 1-33, wherein (c) comprises (i) linking, via non-hydrogen bonds, said subset of said plurality of first polynucleotides with said subset of said plurality of ligation templates, and (ii) linking, via non-hydrogen bonds, said subset of said plurality of second polynucleotides with said subset of said plurality of ligation templates.
Embodiment 35. The method of embodiment 34, wherein said non-hydrogen bonds comprise one or more of a covalent bond, cycloaddition bond, and photocycloaddition bond.
Embodiment 36. The method of any one of embodiments 1-35, wherein a first polynucleotide of said plurality of first polynucleotides comprises a first barcode sequence, and wherein a second polynucleotide of said plurality of second polynucleotides comprises a second barcode sequence different from said first barcode sequence.
Embodiment 37. The method of any one of embodiments 1-35, wherein a second polynucleotide of said plurality of second polynucleotides comprises a barcode sequence.
Embodiment 38. The method of any one of embodiments 1-37, wherein said fluid layer has a thickness of at most about 15 micrometers.
Embodiment 39. A method, comprising: (a) providing a substrate, wherein said substrate comprises a plurality of individually addressable locations comprising a plurality of first polynucleotides immobilized thereto; (b) applying a first fluid layer to said substrate, wherein said first fluid layer comprises (i) a plurality of second polynucleotides comprising a first barcode sequence, and (ii) a first plurality of ligation templates, wherein said first fluid layer has a thickness of at most 50 micrometers; (c) subjecting a first subset of said plurality of individually addressable locations to selective illumination, under conditions sufficient to: (i) link a subset of said plurality of first polynucleotides at said first subset of said plurality of individually addressable locations with a subset of said first plurality of ligation templates hybridized thereto, and (ii) link a subset of said plurality of second polynucleotides with said subset of said first plurality of ligation templates hybridized thereto; (d) applying a second fluid layer to said substrate, wherein said second fluid layer comprises (i) a plurality of third polynucleotides comprising a second barcode sequence, and (ii) a second plurality of ligation templates, wherein said second fluid layer has a thickness of at most 15 micrometers; and (e) subjecting a second subset of said plurality of individually addressable locations to selective illumination, under conditions sufficient to: (i) link a second subset of said plurality of second polynucleotides at said second subset of said plurality of individually addressable locations with a subset of said second plurality of ligation templates hybridized thereto, and (ii) link a subset of said plurality of third polynucleotides with said subset of said second plurality of ligation templates hybridized thereto.
Embodiment 40. The method of embodiment 39, wherein said first plurality of ligation templates and said second plurality of ligation templates have identical sequences.
Embodiment 41. The method of embodiment 39, wherein said first plurality of ligation templates and said second plurality of ligation templates comprise different sequences.
Embodiment 42. The method of any one of embodiments 39-41, wherein said first barcode sequence and said second barcode sequence comprise sequence homology.
Embodiment 43. The method of any one of embodiments 39-41, wherein said first barcode sequence and said second barcode sequence comprise different sequences.
Embodiment 44. The method of any one of embodiments 39-43, wherein said first subset of said plurality of individually addressable locations and said second subset of said plurality of individually addressable locations are mutually exclusive locations.
Embodiment 45. The method of any one of embodiments 39-43, wherein said first subset of said plurality of individually addressable locations and said second subset of said plurality of individually addressable locations are a same set of individually addressable locations.
Embodiment 46. The method of any one of embodiments 39-43, wherein said first subset of said plurality of individually addressable locations and said second subset of said plurality of individually addressable locations comprises at least a common subset of individually addressable locations.
Embodiment 47. The method of any one of embodiments 39-46, wherein said first subset of said plurality of individually addressable locations is selected based on a pre-determined spatial location of said substrate corresponding to said first subset of said plurality of individually addressable locations.
Embodiment 48. The method of any one of embodiments 39-47, wherein said second subset of said plurality of individually addressable locations is selected based on a second pre-determined spatial location of said substrate corresponding to said second subset of said plurality of individually addressable locations.
Embodiment 49. The method of embodiment 47 or 48, wherein said pre-determined spatial location or said second pre-determined spatial location of said substrate is addressed by a Digital Micromirror Device (DMD).
Embodiment 50. The method of any one of embodiments 39-49, further comprising, subsequent to linking in (c), subjecting said substrate to second conditions sufficient for said subset of said plurality of first polynucleotides and said subset of said plurality of second polynucleotides to form a bond.
Embodiment 51. The method of any one of embodiments 39-50, further comprising, subsequent to linking in (e), subjecting said substrate to second conditions sufficient for said second subset of said plurality of second polynucleotides and said subset of said plurality of third polynucleotides to form a bond.
Embodiment 52. The method of embodiment 50 or 51, wherein formation of said bond is catalyzed by a ligase.
Embodiment 53. The method of any one of embodiments 50-52, wherein said bond is a phosphodiester bond.
Embodiment 54. The method of any one of embodiments 50-53, further comprising, subsequent to the formation of said bond, performing an amplification reaction to generate a plurality of amplification products.
Embodiment 55. The method of embodiment 54, further comprising sequencing said plurality of amplification products, or derivatives thereof.
Embodiment 56. The method of any one of embodiments 39-55, wherein said selective illumination in (c) and (e) comprises providing ultraviolet (UV) light.
Embodiment 57. The method of embodiment 56, wherein said UV light comprises a wavelength of about 365 nanometers (nm).
Embodiment 58. The method of any one of embodiments 39-57, further comprising, subsequent to (c), subjecting said plurality of first polynucleotides, said plurality of second polynucleotides, and said first plurality of ligation templates to a second illumination, under conditions sufficient to break a subset of a plurality of links generated in (c) between (i) said subset of said plurality of first polynucleotides and said subset of said first plurality of ligation templates, and (ii) said subset of said plurality of second polynucleotides and said subset of said second plurality of ligation templates.
Embodiment 59. The method of any one of embodiments 39-58, further comprising, subsequent to (e), subjecting said plurality of second polynucleotides, said plurality of third polynucleotides, and said second plurality of ligation templates to a third illumination, under conditions sufficient to break a subset of a plurality of second links generated in (e) between (i) said second subset of said plurality of second polynucleotides and said subset of said second plurality of ligation templates, and (ii) said subset of said plurality of third polynucleotides and said subset of said second plurality of ligation templates hybridized thereto.
Embodiment 60. The method of embodiment 58 or 59, wherein said second illumination comprises UV light.
Embodiment 61. The method of embodiment 60, wherein said UV light comprises a wavelength of about 312 nanometers (nm).
Embodiment 62. The method of any one of embodiments 39-61, wherein a plurality of analytes are immobilized to said plurality of individually addressable locations, and wherein said plurality of first polynucleotides are coupled to said plurality of analytes.
Embodiment 63. The method of embodiment 62, further comprising, prior to (a), (i) providing said substrate, (ii) immobilizing said plurality of analytes at said plurality of individually addressable locations, and (iii) coupling said plurality of first polynucleotides to said plurality of analytes.
Embodiment 64. The method of any one of embodiments 62-63, wherein said plurality of first polynucleotides is coupled to said plurality of analytes via a plurality of analyte-binding moieties of said plurality of first polynucleotides.
Embodiment 65. The method of embodiment 64, wherein said plurality of analyte-binding moieties comprises comprise one or more members selected from the group consisting of: nucleic acids, proteins, lipids, saccharides, polysaccharides, and a combination thereof.
Embodiment 66. The method of embodiment 65, wherein said proteins comprise antibodies or antigen binding fragments thereof.
Embodiment 67. The method of any one of embodiments 62-66, wherein said plurality of analytes comprises one or more members selected from the group consisting of: nucleic acids, proteins, cells, lipids, saccharides, polysaccharides, and a combination thereof.
Embodiment 68. The method of embodiment 67, wherein said proteins comprise one or more of extracellular proteins, cell surface proteins, cell membrane proteins, and intracellular proteins.
Embodiment 69. The method of any one of embodiments 39-61, wherein a plurality of beads are immobilized to said plurality of individually addressable locations, and wherein said plurality of first polynucleotides are coupled to said plurality of beads.
Embodiment 70. The method of any one of embodiments 39-69, wherein (c) comprises (i) cross-linking said subset of said plurality of first polynucleotides with said subset of said first plurality of ligation templates, and (ii) cross-linking said subset of said plurality of second polynucleotides with said subset of said first plurality of ligation templates.
Embodiment 71. The method of embodiment 70, wherein said plurality of first polynucleotides comprises a plurality of cross-linkers used in said cross-linking in (c)(i).
Embodiment 72. The method of embodiment 71, wherein said plurality of cross-linkers comprises 3-Cyanovinylcarbazole nucleosides (CNVKs).
Embodiment 73. The method of any one of embodiments 71 or 72, wherein said cross-linking in (c)(i) comprises forming a cross-link between a cross-linker of said plurality of cross-linkers and a nucleotide of said first plurality of ligation templates.
Embodiment 74. The method of embodiment 73, wherein said nucleotide of said first plurality of ligation templates is a cytosine (C) or a thymine (T).
Embodiment 75. The method of embodiment 74, wherein said nucleotide of said first plurality of ligation templates is a C.
Embodiment 76. The method of embodiment 74, wherein said nucleotide of said first plurality of ligation templates is a T.
Embodiment 77. The method of any one of embodiments 39-76, wherein (c) comprises (i) linking, via non-hydrogen bonds, said subset of said plurality of first polynucleotides with said subset of said first plurality of ligation templates, and (ii) linking, via non-hydrogen bonds, said subset of said plurality of second polynucleotides with said subset of said first plurality of ligation templates
Embodiment 78. The method of embodiment 77, wherein said non-hydrogen bonds comprise one or more of a covalent bond, cycloaddition bond, and photocycloaddition bond.
Embodiment 79. The method of any one of embodiments 39-78, wherein said first fluid layer or said second fluid layer has a thickness of at most about 15 micrometers.
Embodiment 80. A system for barcode generation, comprising: a substrate comprising a plurality of individually addressable locations; a plurality of first polynucleotides immobilized at said plurality of individually addressable locations, wherein said plurality of first polynucleotides comprises a plurality of first cross-linkers; and a fluid layer with a thickness of at most 50 micrometers on said substrate, wherein said fluid layer comprises: a plurality of second polynucleotides, wherein said plurality of second polynucleotides comprises a barcode sequence, wherein said plurality of second polynucleotides comprises a plurality of second cross-linkers; and a plurality of ligation templates, wherein each of said plurality of ligation templates comprises a first nucleotide configured to cross-link with said plurality of first cross-linkers and a second nucleotide configured to cross-link with said plurality of second cross-linkers.
Embodiment 81. The system of embodiment 80, further comprising an illumination system, configured to selectively illuminate one or more subsets of individually addressable locations on said substrate.
Embodiment 82. The system of embodiment 81, wherein said illumination system comprises a Digital Micromirror Device (DMD).
Embodiment 83. The system of any one of embodiments 80-82, wherein at least a subset of said plurality of ligation templates are hybridized to a subset of said plurality of first polynucleotides.
Embodiment 84. The system of any one of embodiments 80-83, wherein at least said subset of said plurality of ligation templates are hybridized to a subset of said plurality of second polynucleotides.
Embodiment 85. The system of any one of embodiments 80-84, wherein a ligation template of said plurality of ligation templates is hybridized to (i) a first polynucleotide of said plurality of first polynucleotides, comprising a first cross-linker of said plurality of first cross-linkers, and (ii) a second polynucleotide of said plurality of second polynucleotides, comprising a second cross-linker of said plurality of second cross-linkers, and wherein said first nucleotide of said ligation template is cross-linked with said first cross-linker.
Embodiment 86. The system of embodiment 85, wherein said second nucleotide of said ligation template is cross-linked with said second cross-linker.
Embodiment 87. The system of any one of embodiments 80-86, further comprising a plurality of analytes immobilized at said plurality of individually addressable locations, wherein said plurality of first polynucleotides are coupled to said plurality of analytes.
Embodiment 88. The system of any one of embodiments 80-86, further comprising a plurality of beads immobilized at said plurality of individually addressable locations, wherein said plurality of first polynucleotides are coupled to said plurality of beads.
Embodiment 89. The system of any one of embodiments 80-88, wherein said fluid layer has a thickness of at most about 15 micrometers.

EXAMPLES

These examples are provided for illustrative purposes only and not to limit the scope of the claims provided herein.

### Example 1

### An Example Method for Photolabile Spatial Labeling Analytes by Exposure to Light

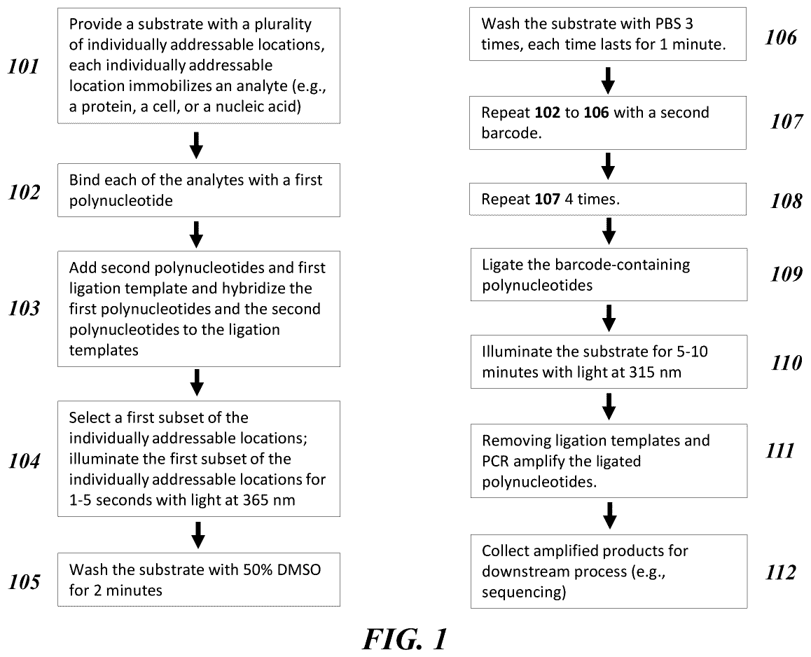

FIG. 1 shows a workflow for an example method for photolabile spatial labeling the analytes. In a first operation 101, a substrate with a plurality of individually addressable locations is provided. Each individually addressable location immobilizes an analyte (e.g., a protein, a cell, a nucleic acid, a saccharide, a polysaccharide, or a combination thereof, etc.). In a second operation 102, a plurality of first polynucleotides is provided to bind at least a subset or each of the analytes. In a third operation 103, the method comprises providing a plurality of second polynucleotides, each comprising a first barcode, and a plurality of first ligation templates. The first polynucleotides and/or the second polynucleotides can hybridize to the ligation templates. In a next operation 104, a first subset of the individually addressable locations is selected and illuminated for 1-5 seconds at 365 nm. The illumination cross-links the ligation template to the second polynucleotide and the first polynucleotide at the selected individually addressable locations. The substrate is washed with 50% DMSO for 2 minutes (operation 105) to remove non-crosslinked polynucleotides and with 1×PBS 3 times to remove DMSO, each time lasting for 1 minute (operation 106). The method further comprises repeating operations 102 to 106 with a third polynucleotide comprising a second barcode (operation 107). In operation 108, operation 107 can be repeated at least one more time (e.g., 4 times) to combine more barcode-containing polynucleotides and ligation templates that can hybridize to the polynucleotide and the immediately preceding polynucleotide (e.g., a second ligation template that can hybridize to both the third polynucleotide and the second polynucleotide). In a next operation 109, the barcode-containing polynucleotides are ligated. The substrate is then illuminated for 5-10 minutes at 315 nm in the next operation 110. This illumination reverses or removes the cross-links formed in operation 104. The ligation templates are removed by washing and the ligated polynucleotide is then PCR amplified in operation 111. In a last operation 112, the amplified products of the ligated polynucleotides are collected for downstream process (e.g., sequencing).

### Example 2

### Ultra-Fast Reversible Photo-Crosslinking of dsDNA

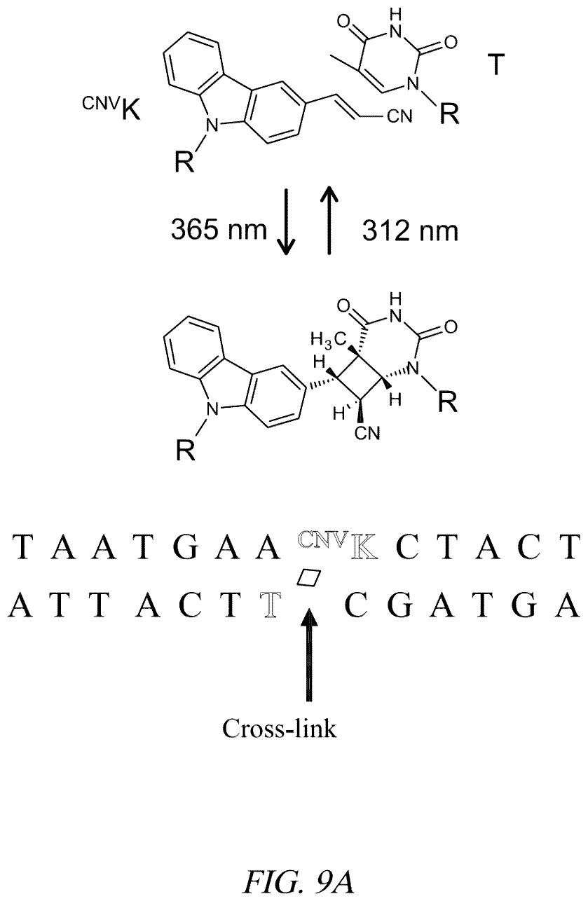

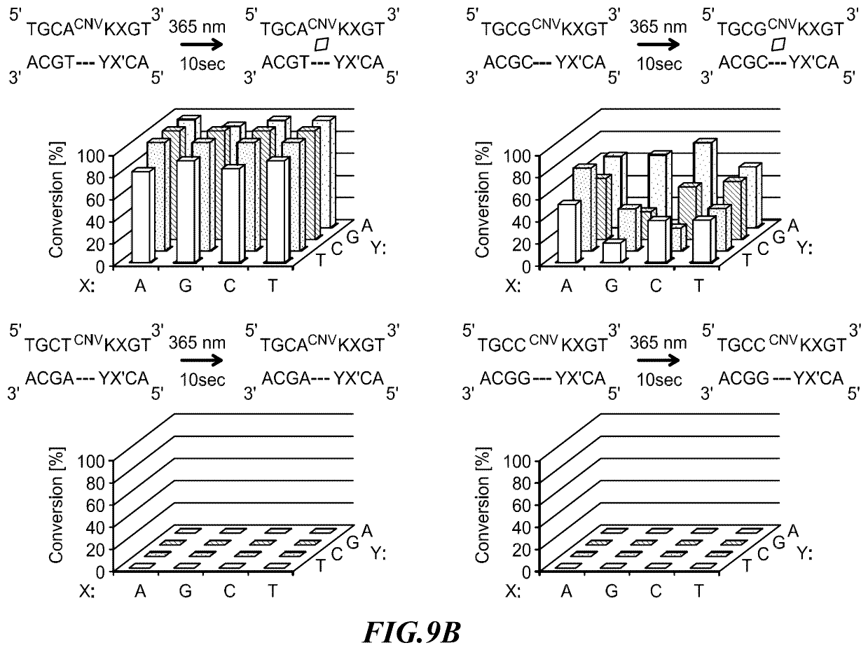

A photolabile cross-linker, such as a nucleoside analogue ^{CNV}K, can enable ultra-fast reversible photo-cross-linking of oligonucleotides. When ^{CNV}K is incorporated into an oligonucleotide strand, a cross-link is formed between ^{CNV}K and a pyrimidine base on the complementary strand when illuminated at 365 nm. A complementary nucleotide on the oligonucleotide strand is in 5′ immediately preceding to ^{CNV}K. FIG. 9A illustrates the photo-crosslinking reaction of ^{CNV}K with thymine (T) in a DNA duplex, which gives a single cross-linked dimer of the oligonucleotide strand and the complementary strand. Complete reversal of the cross-link is achieved by illumination at 312 nm (or at 315 nm). FIG. 9B shows different efficacies of crosslinking reaction with a target pyrimidine for four different DNA nucleotides: thymine (T), cytosine (C), adenine (A), and guanine (G) in various position of the oligonucleotide (i.e., X; X′ is the complementary nucleotide of X) and the complementary strand (i.e., Y). Efficient crosslinking reaction of ^{CNV}K with T and C was observed. The crosslinking reaction of ^{CNV}K did not occur with A and G. Other cross-linkers, such as ^{CNV}D, ^{PC}X, or ^{PCX}D can also be used.

### Example 3

### Selective Illumination Using Digital Micromirror Device (DMD)

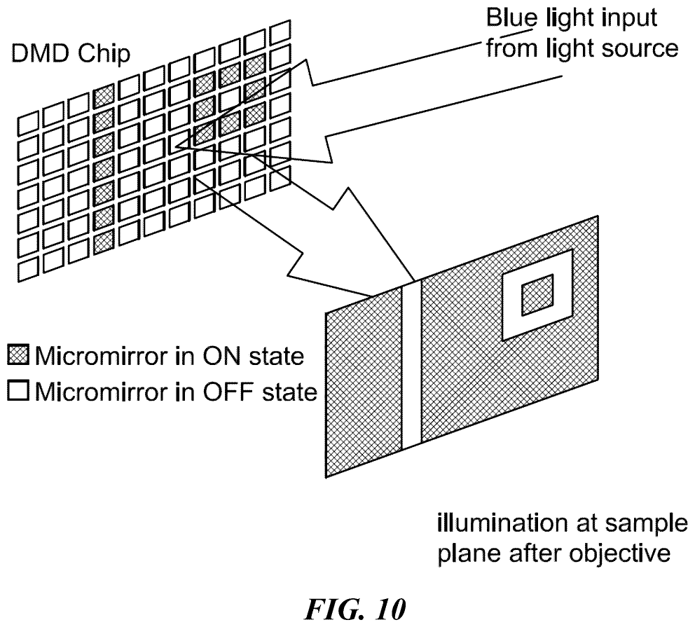

Efficient and selective illumination can be achieved using a Digital Micromirror Device (DMD). FIG. 10 is a schematic representation of a DMD and a DMD illuminated regions of photo-crosslinking reactions. The DMD, comprising an array of micromirrors, allows for selective illumination on the substrate. The micromirror allows for the selective illumination of a selected spatial location of the substrate by blue light input from light source in its ON state, while the micromirror prevents the selective illumination in its OFF state. The DMD device can be used to control selective illumination as described in Examples 1 or 2. The ON and OFF states of the micromirrors are controlled by the computer system described elsewhere in this disclosure.

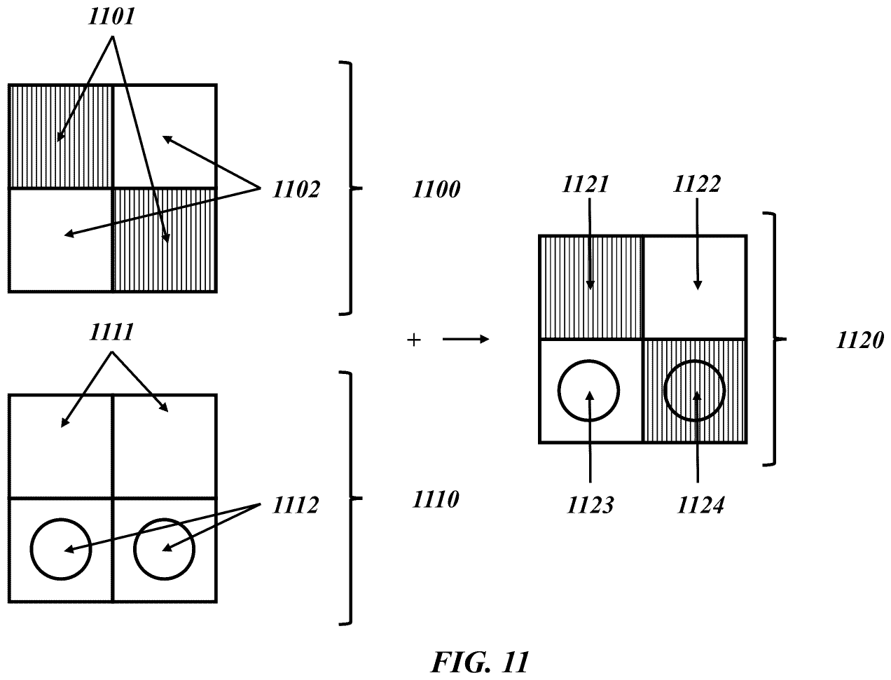

FIG. 11 shows an exemplary schematic illustration of illuminated regions using two independent selective illumination cycles. In a first illumination cycle (1100), a region 1101 is illuminated while a region 1102 is not, resulting in the activation of crosslinking reactions in the region 1101 using the cross-linkers described in this disclosure, such as those of Example 2. In a second illumination cycle (1110), a region 1112 is selectively illuminated while a region 1111 is not, resulting in activation of crosslinking reactions in the region 1112. Subsequent to the two illumination cycles, the resulting substrate 1120 has a region of overlapping illumination (1124), a region of only the first illumination (1121), a region of only the second illumination (1123), and a region of no illumination (1122). Photo-crosslinking of nucleic acids is differentially activated in these locations, resulting in differential cross-linked polynucleotides with different barcodes and various combinations of barcodes in the regions, as described in Example 4. Hence, analytes deposited on these regions are differentially labeled by these different combinations of barcodes.

### Example 4

### Synthesis of Combinations of Barcodes Using Photolabile Cross-Linking

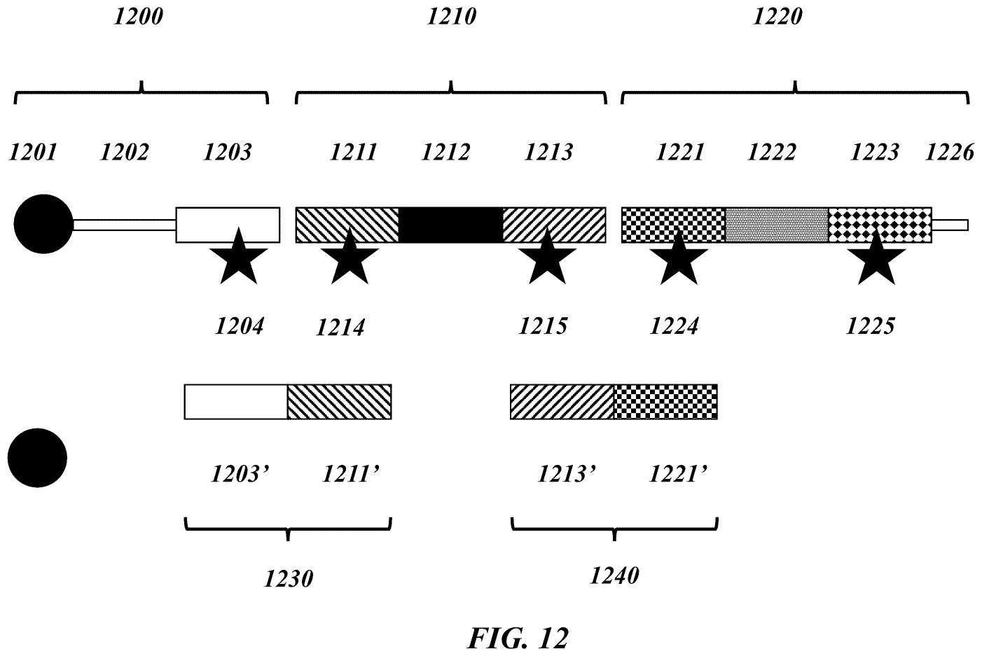

FIG. 12 shows the synthesis of unique barcodes using methods and reagents of the present disclosure. A first polynucleotide 1200 comprises an analyte binding moiety 1201, sequence 1202, and a first hybridization sequence 1203 with a cross-linking moiety 1204. A second polynucleotide 1210 comprises a second hybridization sequence 1211 with a cross-linking moiety 1214, a first barcode sequence 1212, and a third hybridization sequence 1213 with cross-linking moiety 1215. A third polynucleotide 1220 comprises a fourth hybridization sequence 1221 with a cross-linking moiety 1224, a second barcode sequence 1222, and a fifth hybridization sequence 1223 with cross-linking moiety 1225. A first ligation template 1230 comprises sequence 1203′ that is complementary to the first hybridization sequence 1203 and sequence 1211′ that is complementary to the second hybridization sequence 1211. A second ligation template 1240 comprises sequence 1213′ that is complementary to the third hybridization sequence 1213 and sequence 1221′ that is complementary to the fourth hybridization sequence 1221. Upon addition of the first ligation template 1230 to a reaction mixture comprising the first and second polynucleotides (1200 and 1210), sequence 1203′ hybridizes with first hybridization sequence 1203 and sequence 1211′ hybridizes with second hybridization sequence 1211. As described in Example 1, the reaction mixture may then be illuminated for 1-5 seconds at 365 nm to induce cross-links between the first polynucleotide 1200 and the first ligation template 1230 and also between the second polynucleotide 1210 and the first ligation template 1230. The non-crosslinked polynucleotides are removed by washing with DMSO for 2 minutes. Upon addition of the second ligation template 1240 to a reaction mixture comprising the second and third polynucleotides (1210 and 1220), sequence 1213′ hybridizes with third hybridization sequence 1213 and sequence 1221′ can hybridize with fourth hybridization sequence 1221. The reaction mixture is then illuminated at 365 nm to induce cross-links between the second polynucleotide 1210 and the second ligation template 1240 and between the third polynucleotide 1220 and the second ligation template 1240. Non-crosslinked polynucleotides are removed by washing with DMSO. In some instances, the first polynucleotide 1200 may be ligated to the second polynucleotide 1210. In some instances, the second polynucleotide 1210 may be ligated to the third polynucleotide 1220. The ligated product of 1200, 1210, and 1220 contains the barcode combination of 1212 and 1222. 1230 and 1240 are then removed by illumination at 315 nm (to reverse the cross-links) and washing. The ligated product is then PCR amplified using primer sequences that are complementary to sequences 1202 and 1226. The amplified ligation product is then analyzed with further processing (e.g., sequencing).

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Provisional Pat. App. No. 63/291,608, filed Dec. 20, 2021, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A method, comprising:
(a) providing a substrate, wherein said substrate comprises a plurality of individually addressable locations comprising a plurality of first polynucleotides immobilized thereto;
(b) applying a fluid layer to said substrate, wherein said fluid layer comprises (i) a plurality of second polynucleotides and (ii) a plurality of ligation templates, under conditions sufficient to hybridize said plurality of first polynucleotides to said plurality of ligation templates and to hybridize said plurality of second polynucleotides to said plurality of ligation templates, wherein said fluid layer has a thickness of at most 50 micrometers; and
(c) subsequent to hybridization in (b), selectively illuminating a subset of said plurality of individually addressable locations, under conditions sufficient to: (i) link a subset of said plurality of first polynucleotides at said subset of said plurality of individually addressable locations with a subset of said plurality of ligation templates, and (ii) link a subset of said plurality of second polynucleotides with said subset of said plurality of ligation templates.

2. The method of claim 1, further comprising: (d) subsequent to (c), removing a plurality of non-linked second polynucleotides or a plurality of non-linked ligation templates from said substrate.

3. The method of claim 2, wherein said removing in (d) comprises providing to said substrate a solution comprising dimethyl sulfoxide (DMSO), sodium hydroxide (NaOH), or formamide.

4. The method of claim 3, wherein said solution comprises said DMSO at least about 1% by volume in said solution.

5. The method of any one of claims 3-4, further comprising, subsequent to (d), washing said substrate to remove said solution from said substrate.

6. The method of any one of claims 1-5, wherein said subset of said plurality of individually addressable locations is selected based on a pre-determined spatial location of said substrate corresponding to said subset of said plurality of individually addressable locations.

7. The method of claim 6, wherein (c) comprises using a Digital Micromirror Device (DMD) to address said pre-determined spatial location.

8. The method of any one of claims 1-7, further comprising, subsequent to linking in (c), (e) subjecting said substrate to conditions sufficient for said subset of said plurality of first polynucleotides and said subset of said plurality of second polynucleotides to form a bond.

9. The method of claim 8, wherein a ligase catalyzes coupling of said subset of said plurality of first polynucleotides and said subset of said plurality of second polynucleotides.

10. The method of claim 8 or 9, wherein, subsequent to (e), phosphodiester bonds are formed between said subset of said plurality of first polynucleotides and said subset of said plurality of second polynucleotides.

11. The method of any one of claims 8-10, further comprising, subsequent to (e), performing an amplification reaction to generate a plurality of amplification products of said subset of said plurality of first polynucleotides coupled to said subset of said plurality of second polynucleotides.

12. The method of claim 11, further comprising sequencing said plurality of amplification products, or derivatives thereof.

13. The method of any one of claims 1-12, wherein said selectively illuminating in (c) comprises providing ultraviolet (UV) light.

14. The method of claim 13, wherein said UV light comprises a wavelength of about 365 nanometers (nm).

15. The method of any one of claims 1-14, wherein said selectively illuminating in (c) comprises providing UV light for at most about 1 minute.

16. The method of any one of claims 1-15, further comprising, subsequent to said selective illumination in (c), subjecting said plurality of first polynucleotides, said plurality of second polynucleotides, and said plurality of ligation templates to an additional illumination, under conditions sufficient to break a subset of a plurality of links generated in (c) between (i) said subset of said plurality of first polynucleotides and said subset of said plurality of ligation templates, or (ii) said subset of said plurality of second polynucleotides and said subset of said plurality of ligation templates.

17. The method of claim 16, wherein said additional illumination comprises UV light.

18. The method of claim 17, wherein said UV light comprises a wavelength of about 312 nanometers (nm).

19. The method of any one of claims 1-18, wherein a plurality of analytes are immobilized to said plurality of individually addressable locations, and wherein said plurality of first polynucleotides are coupled to said plurality of analytes.

20. The method of claim 19, further comprising, prior to (a), (i) providing said substrate, (ii) immobilizing said plurality of analytes at said plurality of individually addressable locations, and (iii) coupling said plurality of first polynucleotides to said plurality of analytes.

21. The method of any one of claims 19-20, wherein said plurality of first polynucleotides is coupled to said plurality of analytes via a plurality of analyte-binding moieties of said plurality of first polynucleotides.

22. The method of claim 21, wherein said plurality of analyte-binding moieties comprises comprise one or more members selected from the group consisting of: nucleic acids, proteins, lipids, saccharides, polysaccharides, and a combination thereof.

23. The method of claim 22, wherein said proteins comprise antibodies or antigen binding fragments thereof.

24. The method of any one of claims 19-23, wherein said plurality of analytes comprises one or more members selected from the group consisting of: nucleic acids, proteins, cells, lipids, saccharides, polysaccharides, and a combination thereof.

25. The method of claim 24, wherein said proteins comprise one or more of extracellular proteins, cell surface proteins, cell membrane proteins, and intracellular proteins.

26. The method of any one of claims 1-18, wherein a plurality of beads are immobilized to said plurality of individually addressable locations, and wherein said plurality of first polynucleotides are coupled to said plurality of beads.

27. The method of any one of claims 1-26, wherein (c) comprises (i) cross-linking said subset of said plurality of first polynucleotides with said subset of said plurality of ligation templates, and (ii) cross-linking said subset of said plurality of second polynucleotides with said subset of said plurality of ligation templates.

28. The method of claim 27, wherein said plurality of first polynucleotides comprises a plurality of cross-linkers used in said cross-linking in (c)(i).

29. The method of claim 28, wherein said plurality of cross-linkers comprises 3-Cyanovinylcarbazole nucleosides (CNVKs).

30. The method of any one of claim 28 or 29, wherein said cross-linking in (c)(i) comprises forming a cross-link between a cross-linker of said plurality of cross-linkers and a nucleotide of said plurality of ligation templates.

31. The method of claim 30, wherein said nucleotide of said plurality of ligation templates is a cytosine (C) or a thymine (T).

32. The method of claim 31, wherein said nucleotide of said plurality of ligation templates is a C.

33. The method of claim 31, wherein said nucleotide of said plurality of ligation templates is a T.

34. The method of any one of claims 1-33, wherein (c) comprises (i) linking, via non-hydrogen bonds, said subset of said plurality of first polynucleotides with said subset of said plurality of ligation templates, and (ii) linking, via non-hydrogen bonds, said subset of said plurality of second polynucleotides with said subset of said plurality of ligation templates.

35. The method of claim 34, wherein said non-hydrogen bonds comprise one or more of a covalent bond, cycloaddition bond, and photocycloaddition bond.

36. The method of any one of claims 1-35, wherein a first polynucleotide of said plurality of first polynucleotides comprises a first barcode sequence, and wherein a second polynucleotide of said plurality of second polynucleotides comprises a second barcode sequence different from said first barcode sequence.

37. The method of any one of claims 1-35, wherein a second polynucleotide of said plurality of second polynucleotides comprises a barcode sequence.

38. The method of any one of claims 1-37, wherein said fluid layer has a thickness of at most about 15 micrometers.

39. A method, comprising:
(a) providing a substrate, wherein said substrate comprises a plurality of individually addressable locations comprising a plurality of first polynucleotides immobilized thereto;
(b) applying a first fluid layer to said substrate, wherein said first fluid layer comprises (i) a plurality of second polynucleotides comprising a first barcode sequence, and (ii) a first plurality of ligation templates, wherein said first fluid layer has a thickness of at most 50 micrometers;
(c) subjecting a first subset of said plurality of individually addressable locations to selective illumination, under conditions sufficient to: (i) link a subset of said plurality of first polynucleotides at said first subset of said plurality of individually addressable locations with a subset of said first plurality of ligation templates hybridized thereto, and (ii) link a subset of said plurality of second polynucleotides with said subset of said first plurality of ligation templates hybridized thereto;
(d) applying a second fluid layer to said substrate, wherein said second fluid layer comprises (i) a plurality of third polynucleotides comprising a second barcode sequence, and (ii) a second plurality of ligation templates, wherein said second fluid layer has a thickness of at most 15 micrometers; and
(e) subjecting a second subset of said plurality of individually addressable locations to selective illumination, under conditions sufficient to: (i) link a second subset of said plurality of second polynucleotides at said second subset of said plurality of individually addressable locations with a subset of said second plurality of ligation templates hybridized thereto, and (ii) link a subset of said plurality of third polynucleotides with said subset of said second plurality of ligation templates hybridized thereto.

40. The method of claim 39, wherein said first plurality of ligation templates and said second plurality of ligation templates have identical sequences.

41. The method of claim 39, wherein said first plurality of ligation templates and said second plurality of ligation templates comprise different sequences.

42. The method of any one of claims 39-41, wherein said first barcode sequence and said second barcode sequence comprise sequence homology.

43. The method of any one of claims 39-41, wherein said first barcode sequence and said second barcode sequence comprise different sequences.

44. The method of any one of claims 39-43, wherein said first subset of said plurality of individually addressable locations and said second subset of said plurality of individually addressable locations are mutually exclusive locations.

45. The method of any one of claims 39-43, wherein said first subset of said plurality of individually addressable locations and said second subset of said plurality of individually addressable locations are a same set of individually addressable locations.

46. The method of any one of claims 39-43, wherein said first subset of said plurality of individually addressable locations and said second subset of said plurality of individually addressable locations comprises at least a common subset of individually addressable locations.

47. The method of any one of claims 39-46, wherein said first subset of said plurality of individually addressable locations is selected based on a pre-determined spatial location of said substrate corresponding to said first subset of said plurality of individually addressable locations.

48. The method of any one of claims 39-47, wherein said second subset of said plurality of individually addressable locations is selected based on a second pre-determined spatial location of said substrate corresponding to said second subset of said plurality of individually addressable locations.

49. The method of claim 47 or 48, wherein said pre-determined spatial location or said second pre-determined spatial location of said substrate is addressed by a Digital Micromirror Device (DMD).

50. The method of any one of claims 39-49, further comprising, subsequent to linking in (c), subjecting said substrate to second conditions sufficient for said subset of said plurality of first polynucleotides and said subset of said plurality of second polynucleotides to form a bond.

51. The method of any one of claims 39-50, further comprising, subsequent to linking in (c), subjecting said substrate to second conditions sufficient for said second subset of said plurality of second polynucleotides and said subset of said plurality of third polynucleotides to form a bond.

52. The method of claim 50 or 51, wherein formation of said bond is catalyzed by a ligase.

53. The method of any one of claims 50-52, wherein said bond is a phosphodiester bond.

54. The method of any one of claims 50-53, further comprising, subsequent to the formation of said bond, performing an amplification reaction to generate a plurality of amplification products.

55. The method of claim 54, further comprising sequencing said plurality of amplification products, or derivatives thereof.

56. The method of any one of claims 39-55, wherein said selective illumination in (c) and (e) comprises providing ultraviolet (UV) light.

57. The method of claim 56, wherein said UV light comprises a wavelength of about 365 nanometers (nm).

58. The method of any one of claims 39-57, further comprising, subsequent to (c), subjecting said plurality of first polynucleotides, said plurality of second polynucleotides, and said first plurality of ligation templates to a second illumination, under conditions sufficient to break a subset of a plurality of links generated in (c) between (i) said subset of said plurality of first polynucleotides and said subset of said first plurality of ligation templates, and (ii) said subset of said plurality of second polynucleotides and said subset of said second plurality of ligation templates.

59. The method of any one of claims 39-58, further comprising, subsequent to (e), subjecting said plurality of second polynucleotides, said plurality of third polynucleotides, and said second plurality of ligation templates to a third illumination, under conditions sufficient to break a subset of a plurality of second links generated in (e) between (i) said second subset of said plurality of second polynucleotides and said subset of said second plurality of ligation templates, and (ii) said subset of said plurality of third polynucleotides and said subset of said second plurality of ligation templates hybridized thereto.

60. The method of claim 58 or 59, wherein said second illumination comprises UV light.

61. The method of claim 60, wherein said UV light comprises a wavelength of about 312 nanometers (nm).

62. The method of any one of claims 39-61, wherein a plurality of analytes are immobilized to said plurality of individually addressable locations, and wherein said plurality of first polynucleotides are coupled to said plurality of analytes.

63. The method of claim 62, further comprising, prior to (a), (i) providing said substrate, (ii) immobilizing said plurality of analytes at said plurality of individually addressable locations, and (iii) coupling said plurality of first polynucleotides to said plurality of analytes.

64. The method of any one of claims 62-63, wherein said plurality of first polynucleotides is coupled to said plurality of analytes via a plurality of analyte-binding moieties of said plurality of first polynucleotides.

65. The method of claim 64, wherein said plurality of analyte-binding moieties comprises comprise one or more members selected from the group consisting of: nucleic acids, proteins, lipids, saccharides, polysaccharides, and a combination thereof.

66. The method of claim 65, wherein said proteins comprise antibodies or antigen binding fragments thereof.

67. The method of any one of claims 62-66, wherein said plurality of analytes comprises one or more members selected from the group consisting of: nucleic acids, proteins, cells, lipids, saccharides, polysaccharides, and a combination thereof.

68. The method of claim 67, wherein said proteins comprise one or more of extracellular proteins, cell surface proteins, cell membrane proteins, and intracellular proteins.

69. The method of any one of claims 39-61, wherein a plurality of beads are immobilized to said plurality of individually addressable locations, and wherein said plurality of first polynucleotides are coupled to said plurality of beads.

70. The method of any one of claims 39-69, wherein (c) comprises (i) cross-linking said subset of said plurality of first polynucleotides with said subset of said first plurality of ligation templates, and (ii) cross-linking said subset of said plurality of second polynucleotides with said subset of said first plurality of ligation templates.

71. The method of claim 70, wherein said plurality of first polynucleotides comprises a plurality of cross-linkers used in said cross-linking in (c)(i).

72. The method of claim 71, wherein said plurality of cross-linkers comprises 3-Cyanovinylcarbazole nucleosides (CNVKs).

73. The method of any one of claim 71 or 72, wherein said cross-linking in (c)(i) comprises forming a cross-link between a cross-linker of said plurality of cross-linkers and a nucleotide of said first plurality of ligation templates.

74. The method of claim 73, wherein said nucleotide of said first plurality of ligation templates is a cytosine (C) or a thymine (T).

75. The method of claim 74, wherein said nucleotide of said first plurality of ligation templates is a C.

76. The method of claim 74, wherein said nucleotide of said first plurality of ligation templates is a T.

77. The method of any one of claims 39-76, wherein (c) comprises (i) linking, via non-hydrogen bonds, said subset of said plurality of first polynucleotides with said subset of said first plurality of ligation templates, and (ii) linking, via non-hydrogen bonds, said subset of said plurality of second polynucleotides with said subset of said first plurality of ligation templates.

78. The method of claim 77, wherein said non-hydrogen bonds comprise one or more of a covalent bond, cycloaddition bond, and photocycloaddition bond.

79. The method of any one of claims 39-78, wherein said first fluid layer or said second fluid layer has a thickness of at most about 15 micrometers.

80. A system for barcode generation, comprising:
a substrate comprising a plurality of individually addressable locations;
a plurality of first polynucleotides immobilized at said plurality of individually addressable locations, wherein said plurality of first polynucleotides comprises a plurality of first cross-linkers; and
a fluid layer with a thickness of at most 50 micrometers on said substrate, wherein said fluid layer comprises:
a plurality of second polynucleotides, wherein said plurality of second polynucleotides comprises a barcode sequence, wherein said plurality of second polynucleotides comprises a plurality of second cross-linkers; and
a plurality of ligation templates, wherein each of said plurality of ligation templates comprises a first nucleotide configured to cross-link with said plurality of first cross-linkers and a second nucleotide configured to cross-link with said plurality of second cross-linkers.

81. The system of claim 80, further comprising an illumination system, configured to selectively illuminate one or more subsets of individually addressable locations on said substrate.

82. The system of claim 81, wherein said illumination system comprises a Digital Micromirror Device (DMD).

83. The system of any one of claims 80-82, wherein at least a subset of said plurality of ligation templates are hybridized to a subset of said plurality of first polynucleotides.

84. The system of any one of claims 80-83, wherein at least said subset of said plurality of ligation templates are hybridized to a subset of said plurality of second polynucleotides.

85. The system of any one of claims 80-84,
wherein a ligation template of said plurality of ligation templates is hybridized to (i) a first polynucleotide of said plurality of first polynucleotides, comprising a first cross-linker of said plurality of first cross-linkers, and (ii) a second polynucleotide of said plurality of second polynucleotides, comprising a second cross-linker of said plurality of second cross-linkers, and
wherein said first nucleotide of said ligation template is cross-linked with said first cross-linker.

86. The system of claim 85, wherein said second nucleotide of said ligation template is cross-linked with said second cross-linker.

87. The system of any one of claims 80-86, further comprising a plurality of analytes immobilized at said plurality of individually addressable locations, wherein said plurality of first polynucleotides are coupled to said plurality of analytes.

88. The system of any one of claims 80-86, further comprising a plurality of beads immobilized at said plurality of individually addressable locations, wherein said plurality of first polynucleotides are coupled to said plurality of beads.

89. The system of any one of claims 80-88, wherein said fluid layer has a thickness of at most about 15 micrometers.
